(19) United States
(12) Patent Application Publication
(10) Pub. No.: US 2025/0304719 A1
(43) Pub. Date: Oct. 2, 2025

(54) ANTI-TMPRSS6 ANTIBODIES AND USES THEREOF

(71) Applicant: MABWELL THERAPEUTICS INC., San Diego, CA (US)

(72) Inventors: Xin DU, La Jolla, CA (US); Buxin CHEN, San Marcos, CA (US); Yu Jean WANG, San Diego, CA (US)

(21) Appl. No.: 19/042,342

(22) Filed: Jan. 31, 2025

Related U.S. Application Data
(63) Continuation of application No. 17/916,008, filed on Sep. 29, 2022, now Pat. No. 12,215,170.
(60) Provisional application No. 63/006,695, filed on Apr. 7, 2020.
(60) Provisional application No. 63/158,265, filed on Mar. 8, 2021.

(51) Int. Cl. C07K 16/40; A61P 7/06; C12N 15/63

(52) U.S. Cl. CPC C07K 16/40; A61P 7/06; C12N 15/63; C07K 2317/565

(57) ABSTRACT

Antibodies and antigen-binding fragments thereof that bind type II transmembrane serine protease 6 (TMPRSS6) on the surface of a cell and increase hepcidin expression, and methods for treating disorders of iron metabolism using anti TMPRSS6 antibodies and fragments, are provided.

### CROSS-REFERENCE TO RELATED APPLICATIONS

This application is a continuation of U.S. patent application Ser. No. 17/916,008, filed Sep. 29, 2022, as a national stage entry of International Application Number PCT/US2021/025775, filed on Apr. 5, 2021, which claims priority to U.S. Patent Application No. 63/158,265, filed Mar. 8, 2021, and U.S. Patent Application No. 63/006,695, filed Apr. 7, 2020. The contents of the aforementioned applications are incorporated herein by reference in their entireties.

### SEQUENCE LISTING

The instant application contains a Sequence Listing which has been submitted electronically in XML format and is hereby incorporated by reference in its entirety. Said XML copy, created on Jan. 29, 2025, is named “033399-8017 Sequence Listing.xml” and is 137,201 bytes in size.

### FIELD OF THE INVENTION

The present disclosure relates to antibodies and antigen-binding fragments that bind TMPRSS6, and treating disorders of iron metabolism using antibodies and antigen-binding fragments that bind TMPRSS6.

### BACKGROUND

Type II transmembrane serine protease 6 (TMPRSS6) is encoded by the TMPRSS6 gene and primarily expressed in liver. The structure of TMPRSS6 includes a type II transmembrane domain, followed by a sea urchin sperm protein, enteropeptidase and agrin (SEA) domain, a stem region containing two complement factor C1r/C1s, urchin embryonic growth factor and bone morphogenetic protein (CUB) domains and three low-density lipoprotein receptor (LDLR) class A repeats, and a C-terminal trypsin-like serine protease domain (Wang, C.-Y. et al., Front. Pharmacol. 2014. 5:114). Aliases for TMPRSS6 (EC 3.4.21) include: matriptase-2; transmembrane protease serine 6; membrane-bound mosaic serine proteinase matriptase-2; and MT2.

TMPRSS6 plays a significant role in iron homeostasis through the BMP-SMAD signaling pathway that regulates the expression of hepcidin, a hormone that controls iron absorption and mobilization from iron stores. Hepcidin (also known as: HAMP (hepcidin anti-microbial protein or peptide), encoded by HAMP in humans and non-human primates, and Hamp in mice and rats) regulates systemic iron homeostasis by controlling the functional activity of the sole iron efflux channel ferroportin. Hepcidin can lower plasma iron levels by binding to ferroportin and causing internalization and degradation of the complex, thereby preventing iron absorption at the small intestine and release of stored iron. Chronic elevation of hepcidin levels causes systemic iron deficiency, and hepcidin deficiency causes systemic iron overload.

TMPRSS6 negatively regulates the production of hepcidin through a transmembrane signaling pathway that is triggered by iron deficiency and suppresses HAMP activation (Du, X. et al., Science 2008. 320: 1088-1092; Wang, C.-Y. et al., Front. Pharmacol. 2014. 5:114). Low blood iron levels trigger this pathway to reduce hepcidin production, which allows more iron from the diet to be absorbed through the intestines and transported out of storage sites into the bloodstream. In rats under acute iron deprivation, hepatic TMPRSS6 protein levels are upregulated, leading to suppressed hepcidin expression and production (Wang, C.-Y. et al., Front. Pharmacol. 2014. 5:114). Mutations throughout the TMPRSS6 molecule, and especially in the extracellular domain, have been identified in subjects with iron deficiency anemia, in particular iron-refractory iron deficiency anemia (IRIDA) that is unresponsive to oral iron treatment and only partially responsive to parenteral iron therapy (Wang, C.-Y. et al., Front. Pharmacol. 2014. 5:114). Loss-of-function mutations in TMPRSS6 in humans result in elevated levels of hepcidin and iron-deficiency anemia (Camaschella, C., N Engl Journal Med 2013. 168:24) as overproduction of hepcidin leads to defective iron absorption and utilization.

Iron overload disorders result when excess iron accumulates in tissues and organs to an extent that their normal functions are disrupted. Iron toxicity is a common complication of iron overload disorders, leading to high rates of mortality as a result of iron accumulation in major organs. β-thalassemia is an iron overload disorder that occurs when mutations in the HBB gene cause reduced or absent production of β-globin (beta globin) that lead to apoptosis of erythroblasts and a shortage of mature red blood cells, resulting in ineffective erythropoiesis that causes anemia and hyperabsorption of iron leading to iron toxicity. In patients with β-thalassemia, hepcidin is abnormally suppressed in relation to the patient's state of iron loading, creating a hepcidin deficiency that in turn allows excessive iron absorption and development of systemic iron overload. Ineffective erythropoiesis in other disorders such as MDS (myelodysplastic syndrome), dyserythropoietic anemia, sideroblastic anemia, is likewise characterized by low hepcidin leading to iron overload. Hemochromatosis, e.g., hemochromatosis type 1 or hereditary hemochromatosis is an iron overload disorder characterized by excess intestinal absorption of dietary iron and a pathological increase in total body iron stores. Current standards of care for treating iron overload disorders include blood transfusions for ineffective erythropoiesis that can further exacerbate iron overload, iron chelation with poor patient compliance, and phlebotomy or splenectomy to manage symptoms. Therapeutic approaches currently under development include gene therapy targeting the HBB gene, gene therapy and gene editing targeting other relevant genes, hepcidin mimetics, Fc-fusion proteins that target TGF superfamily ligands to inhibit SMAD signaling, antisense RNA drugs targeting TMPRSS6 (e.g., El-Beshlawy A., et al., Blood Cells, Molecules and Diseases 2019. 76: 53-58), and iRNA drugs targeting TMPRSS6.

### SUMMARY

The invention relates to novel antibodies and antigen-binding fragments thereof that bind TMPRSS6, and methods of making and using antibodies and antigen-binding fragments thereof that bind TMPRSS6.

The present disclosure provides anti-TMPRSS6 antibodies, nucleic acids encoding anti-TMPRSS6 antibodies, and methods of making and using anti-TMPRSS6 antibodies. Anti-TMPRSS6 antibodies as disclosed herein encompass anti-TMPRSS6 antibodies and fragments thereof that are capable of binding TMPRSS6. Anti-TMPRSS6 antibodies as disclosed herein are capable of binding to human TMPRSS6 on the surface of a cell expressing human TMPRSS6. The present disclosure provides anti-TMPRSS6 antibodies for therapeutic and diagnostic uses. Anti-TMPRSS6 antibodies as disclosed herein can be used to treat disorders of iron metabolism such as iron overload disorders, in particular β-thalassemias including but not limited to non-transfusion dependent thalassemia, and other disorders of ineffective erythropoiesis.

In one aspect, anti-TMPRSS6 antibodies are provided that are capable of binding to TMPRSS6 on the surface of a cell expressing TMPRSS6 and modulating the activity of at least one component involved in iron metabolism, where a component may be a molecule or a biological process associated with the function of TMPRSS6. In certain embodiments, anti-TMPRSS6 antibodies disclosed herein are capable of modulating the activity of at least one component involved in regulating hepcidin expression. In certain embodiments, anti-TMPRSS6 antibodies disclosed herein are capable of substantially inhibiting TMPRSS6 suppression of hepcidin expression. In certain embodiments, anti-TMPRSS6 antibodies disclosed herein are capable of increasing hepcidin expression. In certain embodiments, anti-TMPRSS6 antibodies disclosed herein are capable of increasing the activity of the hepcidin promoter. In certain embodiments, anti-TMPRSS6 antibodies disclosed herein are capable of substantially inhibiting TMPRSS6 suppression of the BMP/SMAD pathway-induced expression of hepcidin. Anti-TMPRSS6 antibodies disclosed herein may modulate hepcidin expression, including but not limited to substantially inhibiting TMPRSS6 suppression of hepcidin expression, increasing hepcidin expression, increasing hepcidin promoter activity, or substantially inhibiting TMPRSS6 suppression of the BMP/SMAD pathway-induced expression of hepcidin, in a dose-dependent manner. In certain embodiments, anti-TMPRSS6 antibodies disclosed herein are capable of modulating hepcidin expression in a dose-dependent manner. In certain embodiments, anti-TMPRSS6 antibodies disclosed herein are capable of increasing serum hepcidin levels in a dose-dependent manner when administered to a subject. In certain embodiments, anti-TMPRSS6 antibodies disclosed herein are capable of reducing serum iron levels in a dose-dependent manner when administered to a subject. In certain embodiments, anti-TMPRSS6 antibodies disclosed herein are capable of increasing liver hepcidin RNA levels in a dose-dependent manner when administered to a subject. In certain embodiments, anti-TMPRSS6 antibodies disclosed herein are capable of reducing liver non-heme iron, increasing serum hepcidin, increasing liver hepcidin RNA, reducing splenomegaly, increasing red blood count (RBC), increasing hematocrit (HCT), reducing red cell distribution width (RDW), and increased production of mature red cells (increased erythropoiesis) when administered to a subject known or suspected to have an iron overload disorder, in particular a β-thalassemia.

In another aspect, anti-TMPRSS6 antibodies disclosed herein show cross-reactivity with at least one non-human TMPRSS6. In certain embodiments, anti-TMPRSS6 antibodies disclosed herein are capable of binding to at least one non-human TMPRSS6 on the surface of a cell expressing the at least one non-human TMPRSS6. Anti-TMPRSS6 antibodies disclosed herein may be capable of binding human TMPRSS6 and mouse TMPRSS6. Anti-TMPRSS6 antibodies disclosed herein may be capable of binding to human TMPRSS6 and cynomolgus monkey TMPRSS6. Anti-TMPRSS6 antibodies disclosed herein may be capable of binding to each of human TMPRSS6, mouse TMPRSS6, and cynomolgus monkey TMPRSS6.

In another aspect, anti-TMPRSS6 antibodies disclosed herein specifically bind to TMPRSS6 (matriptase-2). In certain embodiments, anti-TMPRSS6 antibodies disclosed herein bind to TMPRSS6 (matriptase-2) and do not show detectable binding to matriptase homologues. In certain embodiments, anti-TMPRSS6 antibodies disclosed herein bind to human TMPRSS6 (matriptase-2) and do not show detectable binding to human matriptase-1 (ST14). In certain embodiments, anti-TMPRSS6 antibodies disclosed herein bind to human TMPRSS6 (matriptase-2) and do not show detectable binding to human matriptase-3 (TMPRSS7). In certain embodiments, anti-TMPRSS6 antibodies disclosed herein bind to human TMPRSS6 (matriptase-2) and do not show detectable binding to either of human matriptase-1 (ST14) or human matriptase-3 (TMPRSS7).

An anti-TMPRSS6 antibody disclosed herein may be a monoclonal antibody, a humanized antibody, a chimeric antibody, a single chain antibody, a Fab fragment, a single-chain variable fragment (scFv), a recombinant antibody, an aptamer, a single-domain antibody (VHH, nanobody), or other TMPRSS6-binding fragment or variant. In certain embodiments, an anti-TMPRSS6 antibody disclosed herein may comprise a framework in which amino acids have been substituted into an existing antibody framework, in particular to influence properties such as antigen-binding ability. In certain embodiments, an anti-TMPRSS6 antibody disclosed herein may comprise complementarity determining regions (CDRs) from a source (parental) antibody that have been grafted (fused) into a framework from a different type (class) of antibody and/or from a different organism than the parental antibody, in particular an acceptor human framework. In certain embodiments, an anti-TMPRSS6 antibody disclosed herein may comprise a framework in which amino acids have been substituted, mutated, or replaced in regions outside of the CDRs to influence properties such as antigen-binding or antibody structure, e.g., in the variable region framework surrounding the CDRs and/or in a constant region, in particular the Fc region. In certain embodiments, one or more of the CDRs have been substituted, mutated, or replaced. In certain embodiments, an anti-TMPRSS6 antibody disclosed herein may be a humanized anti-TMPRSS6 antibody variant.

In certain embodiments, anti-TMPRSS6 antibodies disclosed herein comprise at least one polypeptide having an amino acid sequence as set forth in Table 1, Table 2, or Table 3, or a sequence substantially identical (e.g., at least 85%, 90%, 92%, 95%, 97%, or 98%, 99% identical) to an amino acid sequence as set forth in Table 1, Table 2, or Table 3. Anti-TMPRSS6 antibodies disclosed herein may comprise at least one polypeptide having an amino acid sequence selected from the following, or a sequence substantially identical (e.g., at least 85%, 90%, 92%, 95%, 97%, or 98%, 99% identical) to at least one polypeptide having an amino acid sequence selected from the following: SEQ ID NO: 1; SEQ ID NO: 2; SEQ ID NO: 3; SEQ ID NO: 4; SEQ ID NO: 6; SEQ ID NO: 7; SEQ ID NO: 8; SEQ ID NO: 9; SEQ ID NO: 11; SEQ ID NO: 12; SEQ ID NO: 13; SEQ ID NO: 14; SEQ ID NO: 16; SEQ ID NO: 17; SEQ ID NO: 18; SEQ ID NO: 19; SEQ ID NO: 21; SEQ ID NO: 22; SEQ ID NO: 23; SEQ ID NO: 24; SEQ ID NO: 26; SEQ ID NO: 27; SEQ ID NO: 28; SEQ ID NO: 29; SEQ ID NO: 31; SEQ ID NO: 32; SEQ ID NO: 33; SEQ ID NO: 34; SEQ ID NO: 36; SEQ ID NO: 37; SEQ ID NO: 38; SEQ ID NO: 39; SEQ ID NO: 41; SEQ ID NO: 42; SEQ ID NO: 43; SEQ ID NO: 44; SEQ ID NO: 46; SEQ ID NO: 47; SEQ ID NO: 48; SEQ ID NO: 49; SEQ ID NO: 51; SEQ ID NO: 52; SEQ ID NO: 53; SEQ ID NO: 54; SEQ ID NO: 56; SEQ ID NO: 57; SEQ ID NO: 58; SEQ ID NO: 59; SEQ NO: 61; SEQ ID NO: 63; SEQ ID NO: 65; SEQ ID NO: 67; SEQ ID NO: 69; SEQ ID NO: 71; SEQ ID NO: 73; SEQ ID NO: 75; SEQ ID NO: 77; SEQ ID NO: 79; SEQ ID NO: 81; or SEQ ID NO: 83.

In one embodiment, an anti-TMPRSS6 antibody disclosed herein comprises a heavy chain (HC) variable region polypeptide of the amino acid sequence set forth in SEQ ID NO: 1 or a sequence substantially identical to SEQ ID NO: 1, and a light chain (LC) variable region polypeptide of the amino acid sequence set forth in SEQ ID NO: 6 or a sequence substantially identical to SEQ ID NO: 6. In one embodiment, an anti-TMPRSS6 antibody disclosed herein comprises a heavy chain complementarity determining region 1 (HC CDR1) of the amino acid sequence set forth in SEQ ID NO: 2, a heavy chain complementarity determining region 2 (HC CDR2) of the amino acid sequence set forth in SEQ ID NO: 3, a heavy chain complementarity determining region 3 (HC CDR3) of the amino acid sequence set forth in SEQ ID NO: 4; a light chain complementarity determining region 1 (LC CDR1) of the amino acid sequence set forth in SEQ ID NO: 7, a light chain complementarity determining region 2 (LC CDR2) of the amino acid sequence set forth in SEQ ID NO: 8, and a light chain complementarity determining region 3 (LC CDR3) of the amino acid sequence set forth in SEQ ID NO: 9; or a variant of said antibody comprising 1, 2, 3, 4, 5, or 6 amino acid substitutions in the CDR regions. In one non-limiting embodiment, an anti-TMPRSS6 antibody disclosed herein is the antibody identified herein as MWTx-001, comprising an HC polypeptide having the amino acid sequence set forth in SEQ ID NO: 61 and an LC polypeptide having the amino acid sequence set forth in SEQ ID NO: 63.

In one embodiment, an anti-TMPRSS6 antibody disclosed herein comprises an HC variable region polypeptide of the amino acid sequence set forth in SEQ ID NO: 11 or a sequence substantially identical to SEQ ID NO: 11, and an LC variable region polypeptide of the amino acid sequence set forth in SEQ ID NO: 16 or a sequence substantially identical to SEQ ID NO: 16. In one embodiment, an anti-TMPRSS6 antibody disclosed herein comprises an HC CDR1 of the amino acid sequence set forth in SEQ ID NO: 12, an HC CDR2 of the amino acid sequence set forth in SEQ ID NO: 13, an HC CDR3 of the amino acid sequence set forth in SEQ ID NO: 14; an LC CDR1 of the amino acid sequence set forth in SEQ ID NO: 17, an LC CDR2 of the amino acid sequence set forth in SEQ ID NO: 18, and an LC CDR3 of the amino acid sequence set forth in SEQ ID NO: 19, or a variant of said antibody comprising 1, 2, 3, 4, 5, or 6 amino acid substitutions in the CDR regions. In one non-limiting embodiment, an anti-TMPRSS6 antibody disclosed herein is of the antibody identified herein as MWTx-002, comprising an HC polypeptide having the amino acid sequence set forth in SEQ ID NO: 65 and an LC polypeptide having the amino acid sequence set forth in SEQ ID NO: 67.

In one embodiment, an anti-TMPRSS6 antibody disclosed herein comprises an HC variable region polypeptide of the amino acid sequence set forth in SEQ ID NO: 21 or a sequence substantially identical to SEQ ID NO: 21, and an LC variable region polypeptide of the amino acid sequence set forth in SEQ ID NO: 26 or a sequence substantially identical to SEQ ID NO: 26. In one embodiment, an anti-TMPRSS6 antibody disclosed herein comprises an HC CDR1 of the amino acid sequence set forth in SEQ ID NO: 22, an HC CDR2 of the amino acid sequence set forth in SEQ ID NO: 23, an HC CDR3 of the amino acid sequence set forth in SEQ ID NO: 24; an LC CDR1 of the amino acid sequence set forth in SEQ ID NO: 27, an LC CDR2 of the amino acid sequence set forth in SEQ ID NO: 28, and an LC CDR3 of the amino acid sequence set forth in SEQ ID NO: 29, or a variant of said antibody comprising 1, 2, 3, 4, 5, or 6 amino acid substitutions in the CDR regions. In one non-limiting embodiment, an anti-TMPRSS6 antibody disclosed herein is the antibody identified herein as MWTx-003, comprising an HC polypeptide having the amino acid sequence set forth in SEQ ID NO: 69 and an LC polypeptide having the amino acid sequence set forth in SEQ ID NO: 71.

In one embodiment, an anti-TMPRSS6 antibody disclosed herein comprises an HC variable region polypeptide of the amino acid sequence set forth in SEQ ID NO: 31 or a sequence substantially identical to SEQ ID NO: 31, and an LC variable region polypeptide of the amino acid sequence set forth in SEQ ID NO: 36 or a sequence substantially identical to SEQ ID NO: 36. In one embodiment, an anti-TMPRSS6 antibody disclosed herein comprises an HC CDR1 of the amino acid sequence set forth in SEQ ID NO: 32, an HC CDR2 of the amino acid sequence set forth in SEQ ID NO: 33, an HC CDR3 of the amino acid sequence set forth in SEQ ID NO: 34; an LC CDR1 of the amino acid sequence set forth in SEQ ID NO: 37, an LC CDR2 of the amino acid sequence set forth in SEQ ID NO: 38, and an LC CDR3 of the amino acid sequence set forth in SEQ ID NO: 39, or a variant of said antibody comprising 1, 2, 3, 4, 5, or 6 amino acid substitutions in the CDR regions. In one non-limiting embodiment, an anti-TMPRSS6 antibody disclosed herein is the antibody identified herein as humanized anti-TMPRSS6 antibody variant hzMWTx-001Var, comprising an HC polypeptide having the amino acid sequence set forth in SEQ ID NO: 73 and an LC polypeptide having the amino acid sequence set forth in SEQ ID NO: 75.

In one embodiment, an anti-TMPRSS6 antibody disclosed herein comprises an HC variable region polypeptide of the amino acid sequence set forth in SEQ ID NO: 41 or a sequence substantially identical to SEQ ID NO: 41, and an LC variable region polypeptide of the amino acid sequence set forth in SEQ ID NO: 46 or a sequence substantially identical to SEQ ID NO: 46. In one embodiment, an anti-TMPRSS6 antibody disclosed herein comprises an HC CDR1 of the amino acid sequence set forth in SEQ ID NO: 42, an HC CDR2 of the amino acid sequence set forth in SEQ ID NO: 43, an HC CDR3 of the amino acid sequence set forth in SEQ ID NO: 44; an LC CDR1 of the amino acid sequence set forth in SEQ ID NO: 47, an LC CDR2 of the amino acid sequence set forth in SEQ ID NO: 48, and an LC CDR3 of the amino acid sequence set forth in SEQ ID NO: 49, or a variant of said antibody comprising 1, 2, 3, 4, 5, or 6 amino acid substitutions in the CDR regions. In one non-limiting embodiment, an anti-TMPRSS6 antibody disclosed herein is the antibody identified herein as humanized anti-TMPRSS6 antibody variant hzMWTx-002Var, comprising an HC polypeptide having the amino acid sequence set forth in SEQ ID NO: 77 and an LC polypeptide having the amino acid sequence set forth in SEQ ID NO: 79.

In one embodiment, an anti-TMPRSS6 antibody disclosed herein comprises an HC variable region polypeptide of the amino acid sequence set forth in SEQ ID NO: 51 or a sequence substantially identical to SEQ ID NO: 51, and an LC variable region polypeptide of the amino acid sequence set forth in SEQ ID NO: 56 or a sequence substantially identical to SEQ ID NO: 56. In one embodiment, an anti-TMPRSS6 antibody disclosed herein comprises an HC CDR1 of the amino acid sequence set forth in SEQ ID NO: 52, an HC CDR2 of the amino acid sequence set forth in SEQ ID NO: 53, an HC CDR3 of the amino acid sequence set forth in SEQ ID NO: 54; an LC CDR1 of the amino acid sequence set forth in SEQ ID NO: 57, an LC CDR2 of the amino acid sequence set forth in SEQ ID NO: 58, and an LC CDR3 of the amino acid sequence set forth in SEQ ID NO: 59, or a variant of said antibody comprising 1, 2, 3, 4, 5, or 6 amino acid substitutions in the CDR regions. In one non-limiting embodiment, an anti-TMPRSS6 antibody disclosed herein is the antibody identified herein as humanized anti-TMPRSS6 antibody variant hzMWTx-003Var, comprising an HC polypeptide having the amino acid sequence set forth in SEQ ID NO: 81 and an LC polypeptide having the amino acid sequence set forth in SEQ ID NO: 83.

In another aspect, anti-TMPRSS6 antibodies (including variants and fragments as disclosed herein) are provided that can be used to treat disorders of iron metabolism such as iron overload disorders, in particular β-thalassemia and other disorders of ineffective erythropoiesis. Methods and compositions are provided for using anti-TMPRSS6 antibodies as disclosed herein for therapeutic uses including, but not limited to, treating disorders of iron metabolism such as iron overload disorders, in particular β-thalassemia and other disorders of ineffective erythropoiesis.

In certain embodiments, pharmaceutical compositions comprising an anti-TMPRSS6 antibody disclosed herein and a suitable carrier and/or excipient are provided.

In another aspect, methods for treating a disorder of iron metabolism are provided, such methods comprising administering an effective amount of an anti-TMPRSS6 antibody disclosed herein to a subject in need thereof, wherein administration of the effective amount of anti-TMPRSS6 antibody modulates the activity of a component involved in iron metabolism. In certain embodiments, methods for treating an iron overload disorder comprise administering an effective amount of an anti-TMPRSS6 antibody disclosed herein, wherein administration of the effective amount of anti-TMPRSS6 antibody modulates the activity of a component involved in iron metabolism. In certain embodiments, methods for treating an iron overload disorder comprise administering an effective amount of an anti-TMPRSS6 antibody disclosed herein, wherein administration of the effective amount of anti-TMPRSS6 antibody modulates the activity of at least one component involved in regulating hepcidin expression. In certain embodiments, methods comprise administration of an effective amount of anti-TMPRSS6 antibody that inhibits TMPRSS6 suppression of hepcidin expression. In certain embodiments, administration of the effective amount of anti-TMPRSS6 antibody increases hepcidin expression. In certain embodiments, methods comprise administration of an effective amount of anti-TMPRSS6 antibody that increases the activity of the hepcidin promoter. In certain embodiments, methods comprise administration of an effective amount of anti-TMPRSS6 antibody that inhibits TMPRSS6 suppression of the BMP/SMAD pathway-induced expression of hepcidin. In certain embodiments, methods comprise administration of an effective amount of anti-TMPRSS6 antibody to a subject that results in one or more biological effects associated with an iron overload disorder including but not limited to reducing serum iron, reducing liver non-heme iron, increasing serum hepcidin, increasing liver hepcidin RNA, reducing splenomegaly, increasing red blood count (RBC), increasing hematocrit (HCT), reducing red cell distribution width (RDW), and/or increased production of mature red cells (increased erythropoiesis).

In another aspect, methods for treating a disease or disease state in which abnormal suppression of hepcidin expression is involved are provided, such methods comprising administering an effective amount of an anti-TMPRSS6 antibody disclosed herein to a subject in need thereof, wherein administration of the effective amount of anti-TMPRSS6 antibody modulates the activity of at least one component involved in abnormal suppression of hepcidin expression and reduces abnormal suppression of hepcidin expression. In particular embodiments, the method results in increased hepcidin expression.

In another aspect, methods for treating a disorder of iron metabolism associated with suppressed hepcidin levels are provided, such methods comprising administering an effective amount of an anti-TMPRSS6 antibody disclosed herein to a subject in need thereof, wherein administration of the effective amount of anti-TMPRSS6 antibody modulates the activity of at least one component involved in suppression of hepcidin levels. In certain embodiments, methods comprise administration of an effective amount of anti-TMPRSS6 antibody that increases serum hepcidin levels, increases liver hepcidin RNA, and lowers serum iron levels.

In another aspect, methods are provided for treating disorders of iron metabolism including disorders related to and/or characterized by ineffective erythropoiesis that may include but are not limited to β-thalassemia. In accordance with this aspect, such methods comprise administering an effective amount of an anti-TMPRSS6 antibody disclosed herein to a subject that is known or suspected of having a disorder of iron metabolism related to and/or characterized by ineffective erythropoiesis, wherein administration results in one or more changes related to iron metabolism and/or erythropoiesis in the subject. In certain embodiments, methods are provided wherein administration of the effective amount of anti-TMPRSS6 antibody treats or ameliorates at least one biological effect or symptom associated with the disorder. In particular embodiments, practicing the method results in one or more changes including but not limited to reducing liver non-heme iron, increasing serum hepcidin, increasing liver hepcidin RNA, reducing splenomegaly, increasing red blood count (RBC), increasing hematocrit (HCT), reducing red cell distribution width (RDW), and increased production of mature red cells (increased erythropoiesis).

In another aspect, methods for diagnosing or screening for an iron overload disorder in a subject are provided. In certain embodiments, methods comprise administering anti-TMPRSS6 antibody to a subject known or suspected to have an iron overload disorder and measuring one or more biological effect or symptom associated with an iron overload disorder.

In another aspect, one or more isolated nucleic acid molecules are provided that encode at least a portion of at least one of the anti-TMPRSS6 antibodies disclosed herein. In certain embodiments, isolated nucleic acid molecules that encode at least a portion of at least one of the anti-TMPRSS6 antibodies disclosed herein comprise a nucleotide sequence as set forth in Table 1, Table 2, or Table 3, or a sequence substantially identical (e.g., at least 85%, 90%, 92%, 95%, 97%, or 98%, 99% identical) to a nucleotide sequence as set forth in Table 1, Table 2, or Table 3. In certain embodiments, isolated nucleic acid molecules that encode at least one of the heavy chain (HC) sequences of the anti-TMPRSS6 antibodies disclosed herein may comprise a nucleotide sequence selected from at least one of: SEQ ID NO: 5 or a sequence substantially identical to SEQ ID NO: 5; SEQ ID NO: 15 or a sequence substantially identical to SEQ ID NO: 15; SEQ ID NO. 25 or a sequence substantially identical to SEQ ID NO: 25: SEQ ID NO: 35 or a sequence substantially identical to SEQ ID NO: 35; SEQ ID NO: 45 or a sequence substantially identical to SEQ ID NO: 45; SEQ ID NO: 55 or a sequence substantially identical to SEQ ID NO: 55; SEQ ID NO: 62 or a sequence substantially identical to SEQ ID NO: 62; SEQ ID NO: 66 or a sequence substantially identical to SEQ ID NO: 66; SEQ ID NO: 70 or a sequence substantially identical to SEQ ID NO: 70; SEQ ID NO: 74 or a sequence substantially identical to SEQ ID NO: 74; SEQ ID NO: 78 or a sequence substantially identical to SEQ ID NO: 78, or SEQ ID NO: 82 or a sequence substantially identical to SEQ ID NO: 82. In certain embodiments, isolated nucleic acid molecules that encode at least one of the light chain (LC) sequences of the anti-TMPRSS6 antibodies or antigen-binding fragments thereof disclosed herein may comprise a nucleotide sequence selected from at least one of: SEQ ID NO: 10 or a sequence substantially identical to SEQ ID NO: 10; SEQ ID NO: 20 or a sequence substantially identical to SEQ ID NO: 20; or SEQ ID NO: 30 or a sequence substantially identical to SEQ ID NO: 30; SEQ ID NO: 40 or a sequence substantially identical to SEQ ID NO: 40; SEQ ID NO: 50 or a sequence substantially identical to SEQ ID NO: 50; SEQ ID NO: 60 or a sequence substantially identical to SEQ ID NO: 60; SEQ ID NO: 64 or a sequence substantially identical to SEQ ID NO: 64; SEQ ID NO: 68 or a sequence substantially identical to SEQ ID NO: 68; SEQ ID NO: 72 or a sequence substantially identical to SEQ ID NO: 72; SEQ ID NO: 76 or a sequence substantially identical to SEQ ID NO: 76; SEQ ID NO: 80 or a sequence substantially identical to SEQ ID NO: 80, or SEQ ID NO: 84 or a sequence substantially identical to SEQ ID NO: 84.

In another aspect, vector is provided comprising one or more nucleic acid molecules that encode at least one amino acid sequence of the anti-TMPRSS6 antibodies disclosed herein. In certain embodiments, a vector is provided comprising one or more nucleic acid molecules that encode at least one of the heavy chain (HC) or light chain (LC) sequences of the anti-TMPRSS6 antibodies disclosed herein. In certain embodiments, a vector is provided comprising nucleic acid molecules that encode at least a portion of at least one of the amino acid sequences as set forth in Table 1, Table 2, or Table 3, or at least a portion of an amino acid sequence substantially identical to an amino acid sequence as set forth in Table 1, Table 2, or Table 3. In certain embodiments, a vector is provided comprising nucleic acid molecules that encode at least a portion of at least one of the HC or LC sequences as set forth in Table 1, Table 2, or Table 3, or at least a portion of an amino acid sequence substantially identical to at least one of the HC or LC sequences as set forth in Table 1, Table 2, or Table 3.

In another aspect, at least one host cell is provided containing a vector comprising one or more nucleic acid molecules that encode amino acid sequences of the anti-TMPRSS6 antibodies disclosed herein. In certain embodiments, a host cell is provided containing a vector comprising nucleic acid molecules that encode at least a portion of at least one of the HC or LC sequences as set forth in Table 1, Table 2, or Table 3, or at least a portion of an amino acid sequence substantially identical to at least one of the HC or LC sequences as set forth in Table 1, Table 2, or Table 3. In certain embodiments, at least one host cell is capable of supporting vector expression and recombinant production of anti-TMPRSS6 antibodies or antigen-binding fragments thereof encoded by the vector. In certain embodiments, at least one host cell is capable of supporting vector expression and recombinant production of anti-TMPRSS6 antibodies or antigen-binding fragments thereof encoded by a vector comprising nucleic acid molecules that encode at least a portion of at least one of the HC or LC sequences as set forth in Table 1, Table 2, or Table 3, or at least a portion of an amino acid sequence substantially identical to at least one of the HC or LC sequences as set forth in Table 1, Table 2, or Table 3. In certain embodiments, host cells are transiently transfected with a vector comprising one or more nucleic acid molecules that encode amino acid sequences of the anti-TMPRSS6 antibodies or antigen-binding fragments thereof disclosed herein, wherein the host cells are capable of supporting vector expression and recombinant production of anti-TMPRSS6 antibodies or antigen-binding fragments thereof encoded by the vector.

### BRIEF DESCRIPTION OF THE FIGURES

FIG. 1 shows results from cascade screening of anti-TMPRSS6 antibodies, where antibodies that bind to human TMPRSS6 were assessed using an in vitro functional assay for HAMP promoter activity, and antibodies that showed effects on HAMP promoter activity were assessed for cross-reactivity with non-human TMPRSS6.

FIGS. 2A-2F show effects of anti-TMPRSS6 antibodies on HAMP promoter activity measured by a dual luciferase reporter assay carried out in HepG2 cells, for a range of antibody concentrations. In each plot, open circles represent results using an anti-TMPRSS6 antibody, and open squares represents results using the same concentration of mouse IgG or human IgG1 as a negative (nonspecific binding) control. FIG. 2A shows effects of the MWTx-001 anti-TMPRSS6 antibody on HAMP promoter activity over a range of antibody concentrations. FIG. 2B shows effects of the MWTx-002 anti-TMPRSS6 antibody on HAMP promoter activity over a range of antibody concentrations. FIG. 2C shows effects of the MWTx-003 anti-TMPRSS6 antibody on HAMP promoter activity over a range of antibody concentrations. FIG. 2D shows effects of the hzMWTx-001Var anti-TMPRSS6 antibody on HAMP promoter activity over a range of antibody concentrations. FIG. 2E shows effects of the hzMWTx-002Var anti-TMPRSS6 antibody on HAMP promoter activity over a range of antibody concentrations. FIG. 2F shows effects of the hzMWTx-003Var anti-TMPRSS6 antibody on HAMP promoter activity over a range of antibody concentrations.

FIGS. 3A-3M show results of determinations of binding affinity of anti-TMPRSS6 antibodies. FIGS. 3A-3F show results of determinations of anti-TMPRSS6 antibody binding affinity for human TMPRSS6 expressed on HEK293T cells using two different methods. In each plot, open circles represent results using an anti-TMPRSS6 antibody over a range of concentrations, and open squares represents results using the same concentration of mouse IgG as a negative control. FIGS. 3A-3C show results using cell surface ELISA (measuring HRP-labelled secondary antibody) to measure binding of MWTx-001 (FIG. 3A), MWTx-002 (FIG. 3B), and MWTx-003 (FIG. 3C) to human TMPRSS6, with calculated EC₅₀ values for each antibody used as an estimate of binding affinity. FIGS. 3D-3F show results using FACS (measuring APC-conjugated secondary antibody) to measure binding of MWTx-001 (FIG. 3D), MWTx-002 (FIG. 3E), and MWTx-003 (FIG. 3F) to human TMPRSS6, with calculated EC₅₀ values for each antibody used as an estimate of binding affinity. FIGS. 3G-3M show results of determinations of anti-TMPRSS6 antibody affinity and binding kinetics for human ecto-TMPRSS6-FLAG using the Octet® RED96e with analyte concentrations of 50 nM, 25 nM, 12.5 nM, 6.25 nM, 3.13 nM, 1.56 nM and 0.78 nM. FIG. 3G shows binding kinetics of MWTx-001 anti-TMPRSS6 antibody towards ecto-TMPRSS6-FLAG. FIG. 3H shows binding kinetics of MWTx-002 anti-TMPRSS6 antibody towards ecto-TMPRSS6-FLAG. FIG. 3I shows binding kinetics of MWTx-003 anti-TMPRSS6 antibody towards ecto-TMPRSS6-FLAG. FIG. 3J shows binding kinetics of hzMWTx-001Var anti-TMPRSS6 antibody towards ecto-TMPRSS6-FLAG. FIG. 3K shows binding kinetics of hzMWTx-002Var anti-TMPRSS6 antibody towards ecto-TMPRSS6-FLAG. FIG. 3L shows binding kinetics of hzMWTx-003Var anti-TMPRSS6 antibody towards ecto-TMPRSS6-FLAG. FIG. 3M summaries affinity measurements of all anti-TMPRSS6 antibodies.

FIGS. 4A-4U show results of determinations of cross-reactivity of anti-TMPRSS6 antibodies. FIGS. 4A-4I show results of determinations of the cross-reactivity of anti-TMPRSS6 antibodies MWTx-001, MWTx-002, and MWTx-003 to human TMPRSS6 and non-human TMPRSS6 expressed on HEK293T cells. Each histogram plot shows FACS results for a single antibody incubated with HEK293T cells expressing a TMPRSS6 target (thinner line and lighter fill; indicated with antibody name) and the same antibody incubated with control HEK293T cells that do not express a TMPRSS6 protein (thicker line, darker fill; indicated with Ctrl). FIGS. 4A-4C show results using HEK293T cells stably expressing human TMPRSS6 (HuTMPRSS6-(His)₆) with MWTx-001 (FIG. 4A), MWTx-002 (FIG. 4B), and MWTx-003 (FIG. 4C). FIGS. 4D-4F show results using HEK293T cells stably expressing mouse TMPRSS6 (MoTMPRSS6-(His)₆) with MWTx-001 (FIG. 4D), MWTx-002 (FIG. 4E), and MWTx-003 (FIG. 4F). FIGS. 4G-4I show results using HEK293T cells transiently expressing cynomolgus monkey TMPRSS6 (CynoTMPRSS6-(His)₆) with MWTx-001 (FIG. 4G), MWTx-002 (FIG. 4H), and MWTx-003 (FIG. 4I). FIGS. 4J-4U show results of cross-reactivity of anti-TMPRSS6 antibodies to non-human (mouse (FIGS. 4J, 4L, 4N, 4P, 4R, 4T) or cynomolgus monkey (FIGS. 4K, 4M, 40, 4Q, 4S, 4U)) TMPRSS6 expressed on HEK293T cells using cell surface ELISA (measuring HRP-labelled secondary antibody) to measure binding of MWTx-001 anti-TMPRSS6 antibody (FIGS. 4J-4K), MWTx-002 anti-TMPRSS6 antibody (FIGS. 4L-4M), MWTx-003 anti-TMPRSS6 antibody (FIGS. 4N-40), hzMWTx-001Var anti-TMPRSS6 antibody (FIGS. 4P-4Q), hzMWTx-002Var anti-TMPRSS6 antibody (FIGS. 4R-4S) and hzMWTx-003Var anti-TMPRSS6 antibody (FIG. 4T-4U) to non-human TMPRSS6. In each plot, open circles represent results using an anti-TMPRSS6 antibody, and open squares represents results of mouse IgG or human IgG1 as a negative (nonspecific binding) control, with calculated EC₅₀ values for each antibody used as an estimate of binding affinity.

FIGS. 5A-5R. show results of FACS analysis of binding of anti-TMPRSS6 monoclonal antibodies MWTx-001 (FIGS. 5A-5C), MWTx-002 (FIGS. 5D-5F), MWTx-003 (FIGS. 5G-5I) anti-TMPRSS6 antibodies and their humanized variants hzMWTx-001Var (FIGS. 5J-5L), hzMWTx-002Var (FIGS. 5M-50), hzMWTx-003Var (FIGS. 5P-5R) anti-TMPRSS6 antibodies to HEK293T cells expressing homologous matriptases. HEK293T cells stably expressing human TMPRSS6 (matriptase-2) (FIGS. 5A, 5D, 5G, 5J, 5M, 5P) were used as a positive control, and HEK293T cells over-expressing matriptase (ST14) (FIGS. 5B, 5E, 5H, 5K, 5N, 5Q) and/or matriptase-3 (TMPRSS7) (FIGS. 5C, 5F, 5I, 5L, 5O, 5R) proteins were used to test binding to homologous matriptases. In each panel (FIGS. 5A-5R) HEK293T cells not expressing matriptase (HEK293T) were used as a negative control, with control (Ctrl) results clearly indicated.

FIGS. 6A-6L show anti-TMPRSS6 antibody treatment increases hepcidin expression in mouse in a dose-dependent manner. FIGS. 6A-6C show effects of MWTx-003 anti-TMPRSS6 antibody (FIGS. 6A-6B) or its humanized variant hzMWTx-003Var anti-TMPRSS6 antibody (FIG. 6C) on serum iron. FIG. 6D shows effect of GFP-TMPRSS6 on serum hepcidin. FIGS. 6D-6F show effects of MWTx-003 anti-TMPRSS6 antibody (FIGS. 6D-6E) or its humanized variant hzMWTx-003Var anti-TMPRSS6 antibody (FIG. 6F) on serum hepcidin. FIG. 6G shows effect of GFP-TMPRSS6 on liver hepcidin RNA. FIGS. 6G-61 show effects of MWTx-003 anti-TMPRSS6 antibody (FIGS. 6G-6H) or its humanized variant hzMWTx-003Var anti-TMPRSS6 antibody (FIG. 61) on liver hepcidin RNA. FIGS. 6J-6L show serum concentrations of MWTx-003 anti-TMPRSS6 antibody (FIGS. 6J-6K) or its humanized variant hzMWTx-003Var anti-TMPRSS6 antibody (FIG. 6L). Mouse IgG2b (MoIG2b) (FIGS. 6A-6B, 6D-6E, 6G-6H, 6J-6K) or human IgG1 (HuIGg1)(FIGS. 6C, 6F, 6I, 6L) was used as an isotype control, PBS was used as a vehicle control, and GFP vector was used as a vector control (FIGS. 6A, 6D, 6G, 6J).

FIGS. 7A-7R show in vivo efficacy of anti-TMPRSS6 antibody using a β-thalassemia mouse model. FIGS. 7A-7D show effects of MWTx-003 anti-TMPRSS6 antibody on RBC (FIG. 7A), HGB (FIG. 7B), HCT (FIG. 7C) and RDW (FIG. 7D) using Th3/+ mice. FIG. 7E shows effect of MWTx-003 anti-TMPRSS6 antibody on spleen weight using Th3/+ mice. FIG. 7F shows effect of MWTx-003 anti-TMPRSS6 antibody on serum iron using Th3/+ mice. FIG. 7G shows effect of MWTx-003 anti-TMPRSS6 antibody on liver non-heme iron using Th3/+ mice. FIG. 7H shows effect of MWTx-003 anti-TMPRSS6 antibody on serum hepcidin using Th3/+ mice. FIG. 7I shows effect of MWTx-003 anti-TMPRSS6 antibody on liver hepcidin RNA using Th3/+ mice. FIG. 7J shows serum concentration of MWTx-003 anti-TMPRSS6 antibody using Th3/+ mice. FIGS. 7L-7M show effect of MWTx-003 anti-TMPRSS6 antibody on erythropoiesis using bone marrow from Th3/+ mice. FIGS. 7O-7P show effect of MWTx-003 anti-TMPRSS6 antibody on erythropoiesis using splenocytes from Th3/+ mice. Representative plots in FIGS. 7K-7P show with four distinct cell clusters (I: basophilic erythroblasts; II: polychromatic erythroblasts; III: orthochromatic erythroblasts and nonnucleated reticulocytes and IV: mature red cells) and their corresponding percentages of cell numbers are highlighted. Wildtype mice were used as a positive control (FIGS. 7A-7J, 7K, 7N), and mouse IgG2b (MoIgG2b) was used as isotype control in the treatment (FIGS. 7A-7J, 7L, 7O). Bar graphs in FIGS. 7Q-7R show average results for cell clusters I, II, III, and IV in bone marrow (FIG. 7Q) and spleen (FIG. 7R) for each treatment regime (WT, Th3/+ w/MoIgG2b, Th3/+ w/MWTx-003) after 4 weeks, where comparisons allow identification of shifts in each population, most notably a shift to mature red blood cells (cluster IV) after MWTx-003 treatment.

FIGS. 8A-8D show results of epitope binning of MWTx-001, MWTx-002 and MWTx-003 anti-TMPRSS6 antibodies for human ecto-TMPRSS6-FLAG using the Octet® RED96e. FIG. 8A shows epitope binning of MWTx-001 anti-TMPRSS6 antibody towards ecto-TMPRSS6-FLAG. FIG. 8B shows epitope binning of MWTx-002 anti-TMPRSS6 antibody towards ecto-TMPRSS6-FLAG. FIG. 8C shows epitope binning of MWTx-003 anti-TMPRSS6 antibody towards ecto-TMPRSS6-FLAG. FIG. 8D summarizes association signals for MWTx-001, MWTx-002 and MWTx-003 anti-TMPRSS6 antibodies.

### DETAILED DESCRIPTION

The invention relates to novel antibodies and antigen-binding fragments thereof that bind TMPRSS6, and methods of making and using the same.

### Terminology/Definitions

Scientific and technical terms used in connection with the present invention shall have the meanings that are commonly understood by those of ordinary skill in the art, unless otherwise defined. Use of singular terms (“a” or “an” or “the” or other use of a term in the singular) include plural reference, and plural terms shall include the singular, unless the context clearly dictates otherwise. Thus, for example, reference to “an antibody” includes “one or more” antibodies or a “plurality” of such antibodies.

Generally, nomenclature and techniques of molecular biology, microbiology, cell and tissue culture, protein and nucleotide chemistry, and recombinant DNA techniques available to one of skill of the art can be employed for the antibodies, antigen-binding fragments, compositions, and methods disclosed herein. Techniques and procedures described herein are generally performed according to conventional methods well known in the art and as described in various general and more specific references, inter alia, Sambrook et al. (1989) MOLECULAR CLONING: A LABORATORY MANUAL (2nd ed., Cold Spring Harbor Laboratory Press, Cold Spring Harbor, N.Y.) and Ausubel et al. (1994) CURRENT PROTOCOLS IN MOLECULAR BIOLOGY, Volumes I-III (John Wiley & Sons, N.Y.). Enzymatic reactions and purification techniques are performed according to manufacturer's specifications or as commonly accomplished in the art or as described herein, unless otherwise specified herein. Techniques and methods for pharmaceutical preparation and formulation, and treatment of subjects, are described herein using conventional nomenclature.

“Antibody” refers in the broadest sense to a polypeptide or combination of polypeptides that recognizes and binds to an antigen through one or more immunoglobulin variable regions, where the immunoglobulin variable regions may be naturally occurring or non-naturally occurring, e.g., as a result of engineering, chimerization, humanization, optimization, CDR-grafting, or affinity maturation.

An “antibody” as disclosed herein can be a whole (intact, full length) antibody, a single chain antibody, or an antigen binding fragment with one or two chains, and can be naturally occurring and non-naturally occurring. An antibody comprises at least sufficient complementarity determining regions (CDR), interspersed with framework regions (FR), for the antibody to recognize and bind to an antigen. An anti-TMPRSS6 antibody disclosed herein may be, but is not limited to, at least one of a monoclonal antibody, a polyclonal antibody, a humanized antibody, a chimeric antibody, a single chain antibody, a Fab fragment, a single-chain variable fragment (scFv), an aptamer, a single-domain antibody (VHH or nanobody), a recombinant antibody, a modified antibody having peptide/other moieties attached to antibody and/or additional amino acids added the N- or C-terminus, or other TMPRSS6-binding fragment or variant. Whole antibody, full length antibody, intact antibody, naturally occurring antibody, or equivalent terms are understood to refer to a polypeptide, in particular a glycoprotein, comprising at least two heavy chains (HCs) and two light chains (LCs) interconnected by disulfide bonds. Each HC is comprised of a heavy chain variable region (VH) and an HC constant region (CH), and each light chain is comprised of a light chain variable region (VL) and an LC constant region (CL). The HC and LC variable regions, VH and VL, include a binding domain that interacts with an antigen. The VH and VL regions can be further subdivided into CDR regions characterized by hypervariability, interspersed with FR regions that are typically more conserved. Each VH and VL is typically composed of three CDRs and four FRs arranged from amino-terminus to carboxy-terminus in the following order: FR1, CDR1, FR2, CDR2, FR3, CDR3, FR4. The constant regions of the antibodies may mediate the binding of the immunoglobulin to host tissues or factors, including various cells of the immune system and the classical complement system. Typically, an antibody comprises at least heavy chain (HC) CDR1, CDR2, and CDR3 and light chain (LC) CDR1, CDR2, and CDR3 sequences, where any one of these sequences may be naturally or non-naturally occurring. An antibody may comprise fewer CDR sequences, as long as the antibody can recognize and bind an antigen.

An anti-TMPRSS6 antibody disclosed herein may be a variant comprising at least one altered CDR or framework sequence, wherein CDR and/or framework sequences may by optimized by mutating a nucleic acid molecule encoding such framework sequence. Variants may be constructed with HC and LC portions derived independently from different sources. Techniques for generating variants include but are not limited to conservative amino acid substitution, computer modeling, screening candidate polypeptides alone or in combinations, and codon optimization, and it is understood that a skilled person is capable of generating antibody variants as may be needed. An anti-TMPRSS6 antibody disclosed herein may be a fragment. Antigen binding functions of an antibody can be performed by fragments such as: a Fab fragment; a monovalent fragment consisting of the VL, VH, CL and CHI domains; a F(ab)₂ fragment; a bivalent fragment comprising two Fab fragments linked by a disulfide bridge at the hinge region; an Fd fragment consisting of the VH and CHI domains; a single-chain variable fragment (scFv) consisting of the VL and VH domains of a single arm of an antibody; a single domain antibody (dAb) fragment which consists of a VH domain; and an isolated CDR (VHH, nanobody), or an aptamer. Antigen binding portions can be incorporated into single domain antibodies, maxibodies, minibodies, nanobodies, intrabodies, diabodies, triabodies, tetrabodies, v-NAR and bis-scFv (see, e.g., Hollinger and Hudson, 2005, Nature Biotechnology, 23, 9, 1126-1136). Antigen binding portions of antibodies can be grafted into scaffolds based on polypeptides to form monobodies (see, e.g., U.S. Pat. No. 6,703,199, which describes fibronectin polypeptide monobodies).

The term antibody encompasses various broad classes of polypeptides that can be distinguished biochemically. The “class” of an antibody refers to the type of constant domain or constant region possessed by its heavy chain. Those skilled in the art understand that there are five major classes of antibodies, viz., IgA, IgD, IgE, IgG, and IgM, and several of these may be further divided into subclasses (isotypes), e.g., IgG1, IgG2, IgG3, IgG4, IgA1, and IgA2, each of which is well characterized and known to confer functional specialization. Modified versions of each of these classes and isotypes are readily discernable and within the scope of the instant disclosure. While all immunoglobulin classes are within the scope of the present disclosure, the present disclosure will be directed largely to the IgG class of immunoglobulin molecules.

The term “chimeric” antibody refers to an antibody in which a portion of the heavy chain (HC) and/or light chain (LC) involved in forming the immunoreactive site is derived from a particular source or species, while the remainder of the HC and/or LC is derived from a different source or species. In certain embodiments the target binding region or site will be from a non-human source (e.g., mouse or non-human primate) and the constant region is human.

As used herein, the phrase “humanized antibody” refers to an antibody or antibody variant derived from a non-human antibody, typically a mouse monoclonal antibody, where CDRs from the parental, non-human antibody are grafted (fused) in a framework comprising variable regions derived from a human immunoglobulin framework, in particular an acceptor human framework or a human consensus framework. Techniques and principles for designing, making, and testing humanized antibodies are known (Jones P T, Dear P H, Foote J, Neuberger M S, Winter G. Replacing the complementarity-determining regions in a human antibody with those from a mouse. Nature. 1986 May 29-Jun. 4; 321(6069):522-5; Almagro J C, Fransson J. Humanization of antibodies. Front Biosci. 2008 Jan. 1; 13:1619-33). It is understood that changes can be made to an acceptor framework at multiple locations in order to develop a humanized antibody having improved features according to the desired use, e.g., high affinity for target, low clearance, low toxicity, etc. An anti-TMPRSS6 antibody disclosed herein may be a humanized variant.

“Affinity” refers to the strength of the sum total of noncovalent interactions between a single binding site of a molecule (e.g., an antibody) and its binding partner (e.g., an antigen). Unless indicated otherwise, binding affinity as used herein refers to intrinsic binding affinity which reflects a 1:1 interaction between members of a binding pair (e.g., antibody and antigen). Affinity can be measured by common methods known in the art, including those described herein. The calculated concentration at which approximately 50% of maximal binding (the calculated EC₅₀) can be used as an estimate of affinity. The affinity of a molecule X for its partner Y can generally be represented by the dissociation constant (Kd or KD, representing k_{off}/kₒₙ measured for the interaction).

A “subject” is a mammal, where mammals include but are not limited to primates (e.g., humans and non-human primates such as monkeys), domesticated animals (e.g., cows, sheep, cats, dogs, pigs, llamas, and horses), rabbits, and rodents (e.g., mice and rats). In certain embodiments, the subject is a human. The phrases “to a subject in need thereof” or “to a patient in need thereof” or “to a patient in need of treatment” or “a subject in need of treatment” may include subjects that would benefit from administration of the anti-TMPRSS6 antibodies disclosed herein, for treatment of an iron overload disorder. It is understood that administration of anti-TMPRSS6 antibodies encompasses administration to “a subject in need thereof” can be interpreted as referring to a subject known or suspected to have an iron overload disorder, in particular a β-thalassemia, based on indicators such as symptoms, family history, or genotype. It is further understood that anti-TMPRSS6 antibodies can be administered to a subject that is not known or suspected to have a disorder of iron metabolism, for purposes that may include but are not limited to, preventative or prophylactic purposes, for screening, for diagnostics, for research purposes, or to achieve results distinct from treating a disorder.

An “effective amount” of an anti-TMPRSS6 antibody, e.g., in a pharmaceutical formulation, refers to an amount effective, at dosages and for periods of time necessary, to achieve the desired therapeutic or prophylactic result. It is understood that “effective amount” is intended to refer to the amount of an anti-TMPRSS6 antibody or a pharmaceutical composition comprising an anti-TMPRSS6 antibody that will elicit the biological response of, or desired therapeutic effect on, a cell, a tissue, a system, a non-human animal subject, a non-human mammal subject, or a human subject that is being measured. The terms “therapeutically effective amount”, “pharmacologically effective amount”, and “physiologically effective amount” are used interchangeably to refer to the amount of an anti-TMPRSS6 antibody that is needed to provide a threshold level of active agents in the bloodstream or in the target tissue.

The precise amount will depend upon numerous factors, e.g., the particular anti-TMPRSS6 antibody (active agent), the components and physical characteristics of the composition, intended population of subjects/patients to be treated, considerations such as the disease state, age, sex, and weight of a subject, and the like, and can readily be determined by one skilled in the art, based upon the information provided herein or otherwise available in the relevant literature. The terms, “improve”, “increase” or “reduce”, as used in this context, indicate values or parameters relative to a baseline measurement, such as a measurement in the same subject prior to initiation of the treatment described herein, or a measurement in a control individual (or multiple control individuals) in the absence of the treatment described herein.

The term “pharmaceutical composition” or “pharmaceutical formulation” refers to a preparation which is in such form as to permit the biological activity of an active ingredient contained therein to be effective, in particular an anti-TMPRSS6 antibody. It is understood that a pharmaceutical composition may contain more than one active ingredient, e.g., more than one anti-TMPRSS6 antibody, or a combination of an anti-TMPRSS6 antibody with another active ingredient that acts on a different target, where such combinations can be but are not limited to, a combination of an antiTMPRSS6 antibody with another active ingredient having a desired effect on hematopoietic processes, in particular erythropoiesis, a combination of an anti-TMPRSS6 antibody with gene therapy agents such as agents to carry out gene therapy targeting the HBB gene, or a combination of an anti-TMPRSS6 antibody with Fc-fusion proteins that target TGF superfamily ligands to stimulate erythropoiesis. A “pharmaceutically acceptable carrier” refers to an ingredient in a pharmaceutical formulation, other than an active ingredient, which is nontoxic to a subject. It is understood that a pharmaceutically acceptable carrier can be, but is not limited to, a buffer, excipient, stabilizer, an adjuvant, or preservative.

The term “treat” or “treating” or similar terms as used herein, can refer to an outcome that is deemed beneficial for a particular subject in a defined set of circumstances. Treating a disorder of iron metabolism may refer non-exclusively to any of reducing, ameliorating, slowing, interrupting, arresting, alleviating, stopping, or reversing the progression or severity of an existing symptom, disorder, condition, or disease, and may further encompass prevention or delay of the onset of one or more symptoms of an iron overload disorder, and/or lessening of the severity or frequency of one or more symptoms of an iron overload disorder. The terms “treating” or “method of treating” or equivalents can encompass one or more uses of anti-TMPRSS6 antibodies disclosed herein, including but not limited to therapeutic, prophylactic, preventive, diagnostic, imaging, and screening uses.

The term “vector,” as used herein, refers to a nucleic acid molecule capable of propagating a nucleic acid to which the vector sequence is linked, in a host cell in which the vector is introduced. Vectors capable of directing the expression of nucleic acids to which they are operatively linked are referred to herein as “expression vectors.”

### Anti-TMPRSS6 Antibodies

Antibodies and antigen-binding fragments are provided that are capable of binding TMPRSS6 on the surface of a cell and modulating the activity of at least one component involved in iron metabolism, in particular at least one component involved in iron overload disorders associated with abnormal suppression of hepcidin expression. Anti-TMPRSS6 antibodies that are capable of binding TMPRSS6 on the surface of a cell and modulating the activity of at least one component involved in regulating hepcidin expression can be used in methods for treating iron overload disorders associated with abnormal suppression of hepcidin expression. Anti-TMPRSS6 antibodies that are capable of binding TMPRSS6 on the surface of a cell and modulating TMPRSS6 suppression of hepcidin expression can be used to therapeutically target TMPRSS6 in methods for treating iron overload disorders associated with abnormal suppression of hepcidin expression.

Once antibodies or fragments specific for TMPRSS6, in particular human TMPRSS6 expressed on the surface of a cell, have been obtained, the desired biological activity of modulating the activity of at least one component involved in iron metabolism thereof can be tested by several methods known to the skilled person.

It is understood that “modulate” or “modulating” or similar terms as used herein can refer to one or more effects that can result when an anti-TMPRSS6 antibody disclosed herein binds its target. “Modulating” and its equivalents can refer to different modes of action and effects depending on the component under consideration, i.e., modulating can refer to neutralizing, reversing, inhibiting, blocking, reducing, antagonizing, or otherwise interfering with the activity of certain components involved in iron metabolism, while for other components involved in iron metabolism the term modulating can refer to increasing, enhancing, or having an agonist effect on these components.

It is understood that the term “component” can refer not only to target molecule TMPRSS6, but also to a downstream process or pathway involved in iron metabolism. Thus, a component within the meaning of a process or pathway can be, but is not limited to, regulation of hepcidin expression, TMPRSS6 suppression of hepcidin expression, the process of hepcidin expression, regulation of hepcidin levels, increasing hepcidin levels, the activity of the hepcidin promoter, or TMPRSS6 suppression of the BMP/SMAD pathway-induced expression of hepcidin, regulation of liver non-heme iron levels, one or more processes involved in splenomegaly, or one or more hematopoietic processes involved in regulation of red blood count (RBC), hematocrit (HCT), red cell distribution width (RDW), and erythropoiesis, in particular production of mature red cells.

Anti-TMPRSS6 antibodies as disclosed herein can be used to therapeutically target at least one component involved in iron metabolism, in particular at least one component involved in iron overload disorders. In certain embodiments, anti-TMPRSS6 antibodies as disclosed herein can be used to therapeutically target at least one component involved in regulating hepcidin expression, and modulate the activity of the component to achieve increased hepcidin expression. In certain embodiments, anti-TMPRSS6 antibodies as disclosed herein can be used to modulate the activity of the hepcidin promoter to achieve increased hepcidin expression. It is understood that anti-TMPRSS6 antibodies as disclosed herein can be used to therapeutically target TMPRSS6 and thereby modulate the downstream activity of other components of hepcidin expression, including but not limited to, regulation of liver non-heme iron levels, one or more processes involved in splenomegaly, or one or more hematopoietic processes involved in regulation of red blood count (RBC), hematocrit (HCT), red cell distribution width (RDW), and erythropoiesis, in particular production of mature red cells.

Using anti-TMPRSS6 antibodies as disclosed herein to therapeutically target at least one component involved in iron metabolism, allows precise modulation of the targeted component. It is understood that by using anti-TMPRSS6 antibodies as disclosed herein to precisely target TMPRSS6 and its downstream effects on at least one component involved in regulating hepcidin expression, it is possible to avoid undesirable effects, difficulties with delivery and/or effectiveness, and regulatory hurdles associated with other approaches to treating iron overload disorders that are currently in use or under development, e.g., blood transfusions that can further exacerbate iron overload, iron chelation with poor patient compliance, intrusive phlebotomy or splenectomy that only manage symptoms, gene therapy targeting the HBB gene with potential permanent pleiotropic effects in multiple systems, gene therapy and gene editing with unknown off-target effects, Fc-fusion proteins targeting TGF superfamily ligands to inhibit SMAD signaling that do not reduce the need for iron chelation therapy to manage iron overload, and other approaches that are difficult to control or deliver such as hepcidin mimetics, and antisense or iRNA drugs targeting TMPRSS6. It is understood that using anti-TMPRSS6 antibodies for precise therapeutic targeting does not exclude the possibility of using anti-TMPRSS6 antibodies in methods and compositions for combination treatments, e.g., in combination with another active ingredient that acts on a different target, in combination with an antibody that binds a different target, in combination with gene therapy agents and methods for targeting the HBB gene, or in combination with Fc-fusion proteins that target TGF superfamily ligands to stimulate erythropoiesis.

Anti-TMPRSS6 antibodies disclosed herein allow the development of treatments that can be tailored to each subject (e.g., dosage, frequency of administration), where they can be continued and discontinued with ease, and combined with other therapies. In certain strategic embodiments, anti-TMPRSS6 antibodies disclosed herein can be combined with other therapies that may address multiple therapeutic targets and/or address deficits or undesirable effects of one of the therapies in the combination therapy.

### Exemplary Embodiments of Anti-TMPRSS6 Antibodies and Uses Thereof

Non-limiting exemplary embodiments of anti-TMPRSS6 antibodies of the invention are presently disclosed, in particular in the Examples, Tables, and Figures.

### Antibodies Capable of Binding TMPRSS6

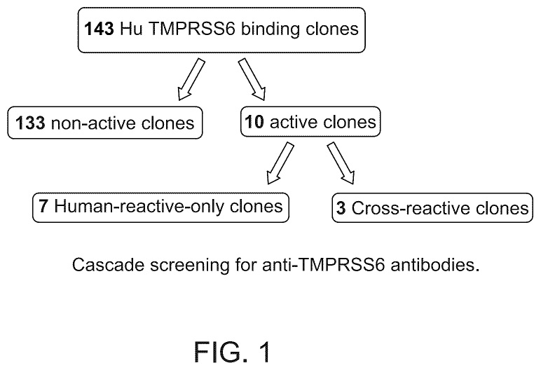

As demonstrated in the Examples, a functional cascade can be used to identify and characterize anti-TMPRSS6 antibodies of the present invention, where a first step in the cascade involves screening for antibodies capable of binding to human TMPRSS6 on the surface of a cell expressing TMPRSS6 (Example 1, FIG. 1), followed by a second step to identify antibodies capable of binding to human TMPRSS6 on the surface of a cell expressing TMPRSS6 and modulating the activity of a component involved in iron metabolism, in this case testing for the ability to increase hepcidin (HAMP) promoter activity (Example 2). As demonstrated by exemplary embodiments shown in FIG. 1, the first step identified 143 antibodies (clones) capable of binding to human TMPRSS6 on the surface of a cell expressing TMPRSS6, and the second step identified ten (10) of the antibodies (out of 143 screened) as “active” antibodies (clones) that were able to increase hepcidin (HAMP) promoter activity.

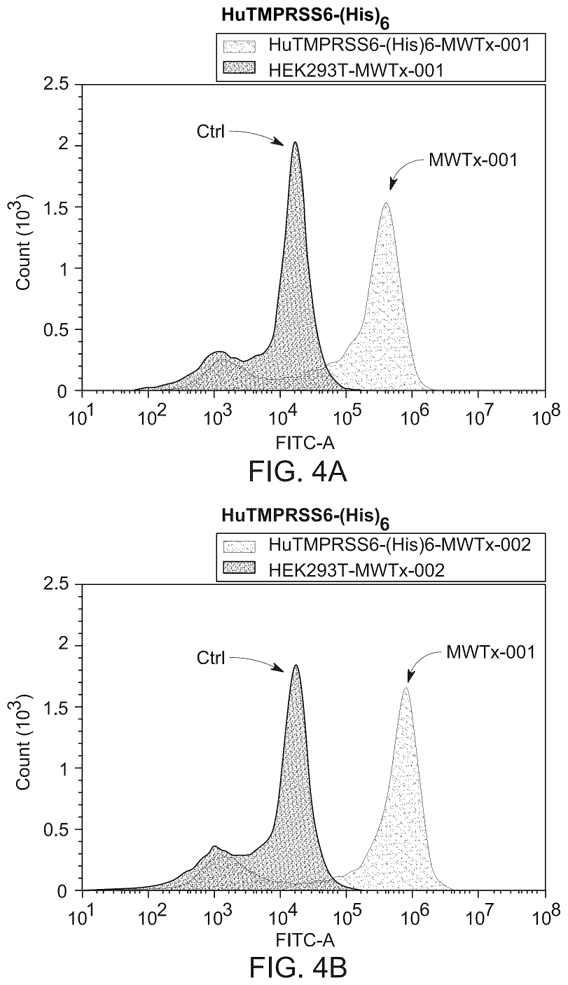

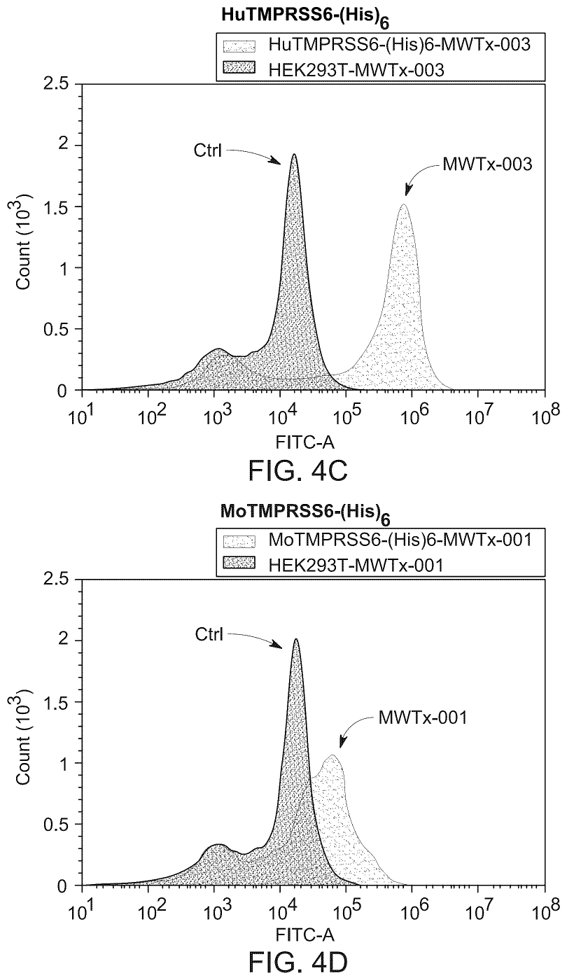

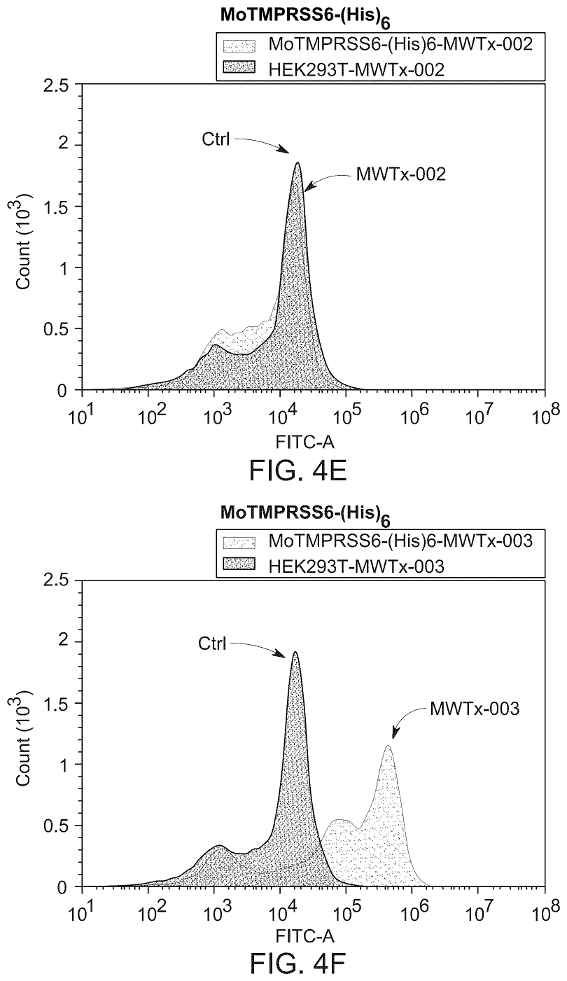

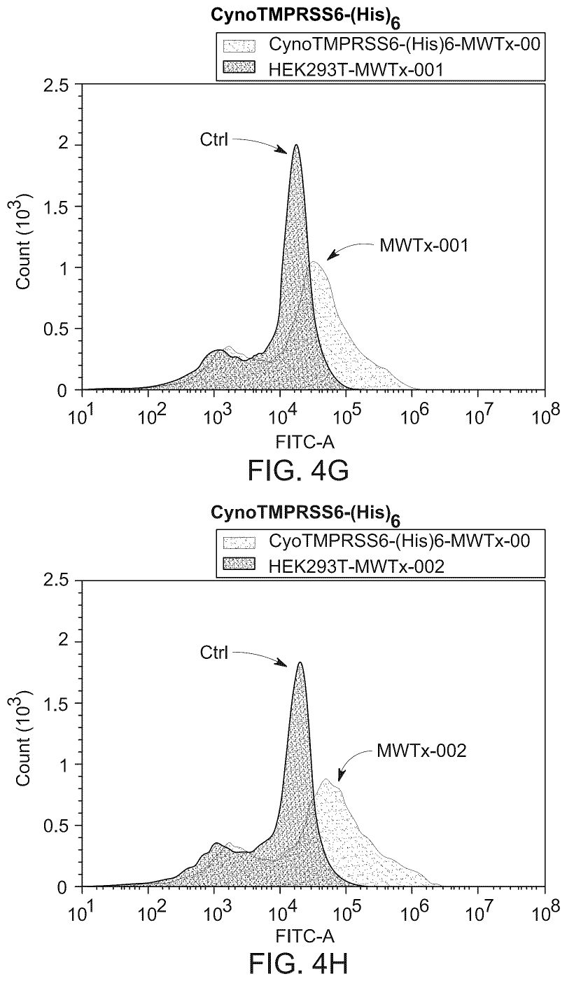

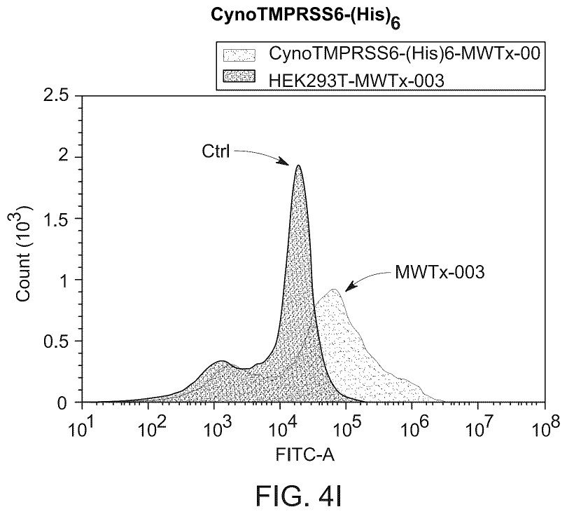

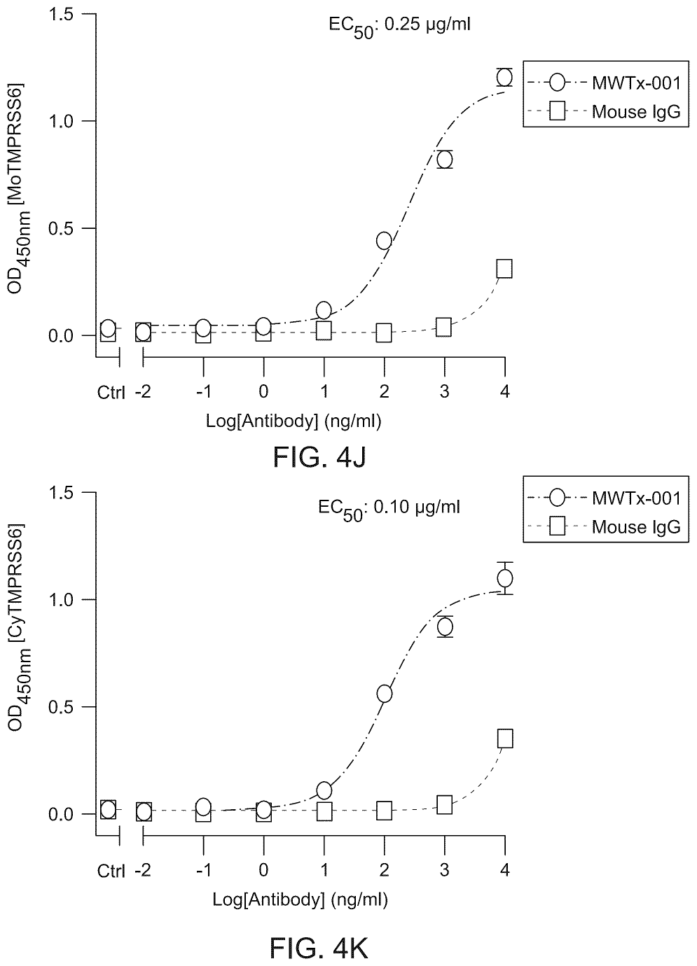

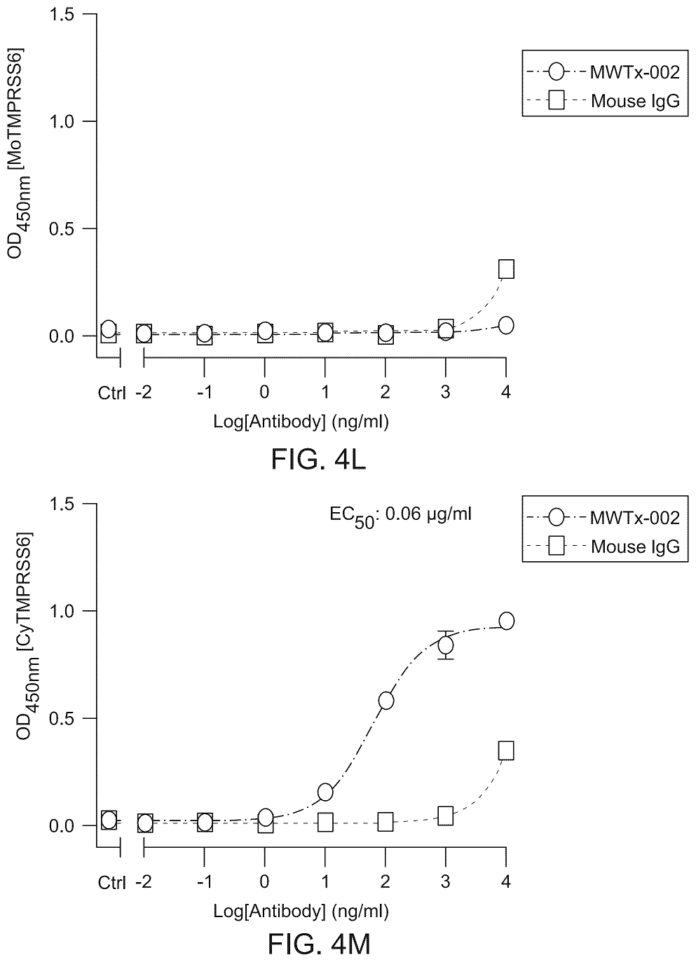

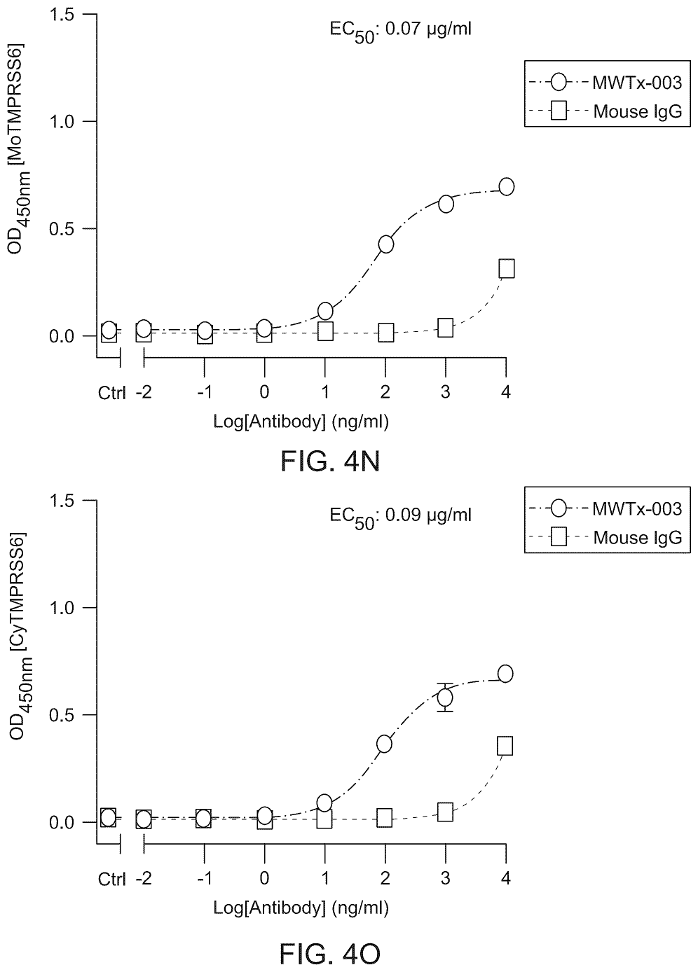

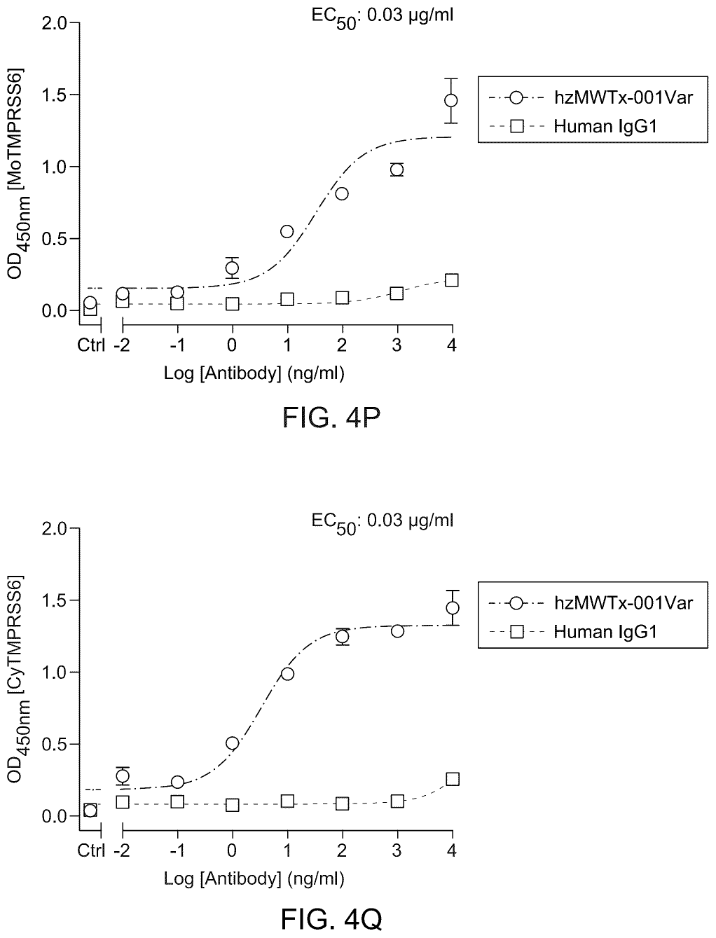

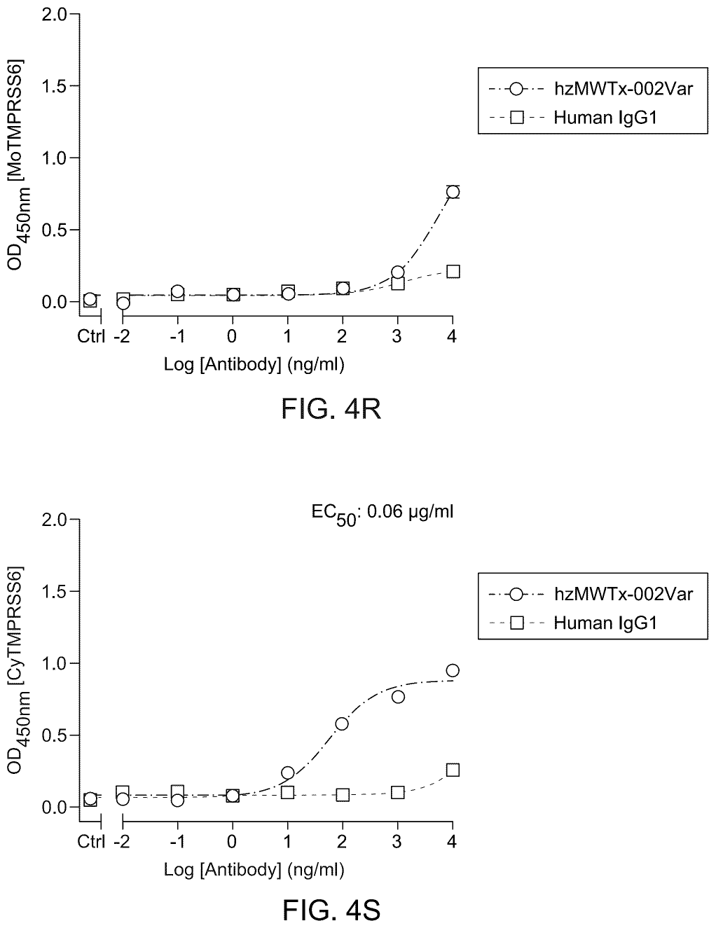

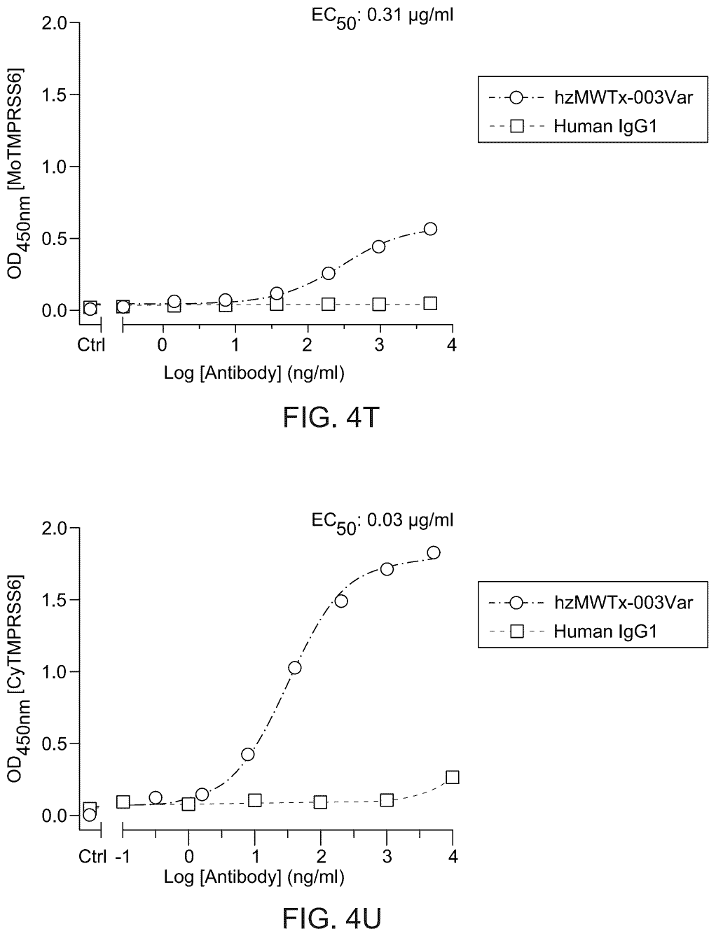

In a third step of the functional cascade (FIG. 1), the ten (10) “active” antibodies were tested for cross-reactivity with non-human TMPRSS6 targets from sources that would be relevant for further studies, viz., testing for cross-reactivity with mouse TMPRSS6 relevant to preclinical efficacy studies in a mouse model, and testing for cross-reactivity with cynomolgus monkey TMPRSS6 relevant to toxicity (safety) trials. As demonstrated by exemplary embodiments shown in FIG. 1, demonstrated in Example 4 and illustrated in FIG. 4, three (3) clones (out of 10 screened) showed cross-reactivity with at least one non-human TMPRSS6 and were designated MWTx-001, MWTx-002, and MWTx-003. Each of the monoclonal antibodies was sequenced and CDRs on each HC and LC were identified (Kabat numbering). HC and LC sequences were identified as follows for: MWTx-001 (SEQ ID NOs: 61 (HC) and 63 (LC)); MWTx-002 (SEQ ID NOs: 65 (HC) and 67 (LC)); and MWTx-003 9SEQ ID NOs: 69 (HC) and 71 (LC)). A hybridoma cell line producing the MWTx-001 monoclonal antibody has been deposited under ATCC Accession No. PTA-126759. A hybridoma cell line producing the MWTx-002 monoclonal antibody has been deposited under ATCC Accession No. PTA-126760. A hybridoma cell line producing the MWTx-003 monoclonal antibody has been deposited under ATCC Accession No. PTA-126761.

### Humanized Variants

Humanized antibodies comprising CDRs derived from a non-human source grafted into a human-derived antibody framework are expected to be non-immunogenic when administered to a human subject. As demonstrated by exemplary embodiments disclosed in Example 2, humanized anti-TMPRSS6 antibody variants were successfully generated, tested, optimized, and selected. Multiple candidate HC and LC variants were developed wherein each HC or LC variant had the same CDR sequences but the variable region frameworks sequences could vary at over 90% of the framework positions, and these variants tested in different HC/LC combinations to identify combinations having desired features. After initial design and testing, variants that showed desired antigen binding affinity were selected for further evaluation and development, including but not limited to modification of some parental CDR sequences to avoid potential unwanted events such as aspartate isomerization, and modification of some constant regions (Fc) to achieve desired functions such as minimizing antibody-dependent cellular cytotoxicity (ADCC), to arrive at humanized variants hzMWTx-001Var (SEQ ID NOs: 73 (HC) and 75 (LC)), hzMWTx-002Var (SEQ ID NOs: 77 (HC) and 79 (LC)), and hzMWTx-003Var (SEQ ID NOs: 81 (HC) and 83 (LC)).

### Anti-TMPRSS6 Antibodies that Increase Hepcidin Promoter Activity

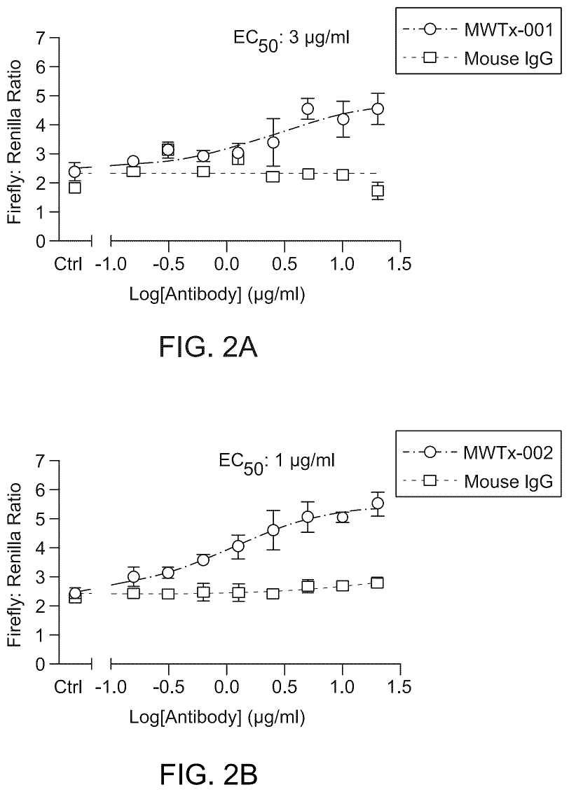

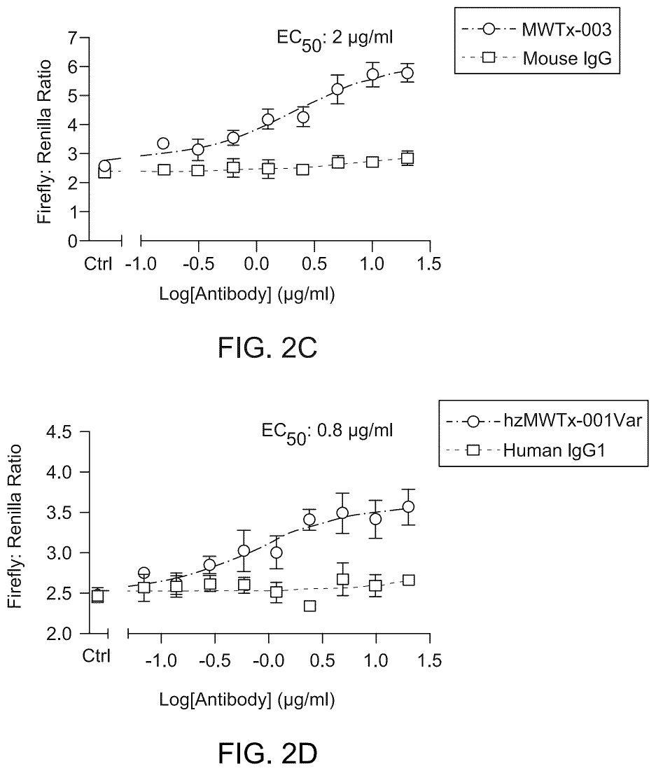

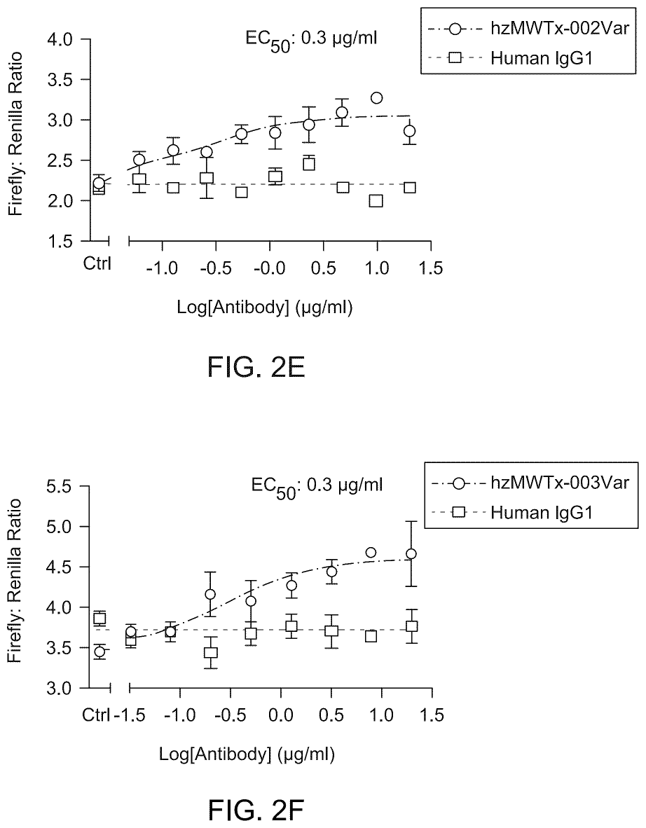

As disclosed herein, antibodies for use in treating iron overload disorders characterized by reduced hepcidin expression may modulate the activity of at least one component involved in hepcidin expression, where the component may be activity of the hepcidin promoter. As demonstrated by exemplary embodiments using an in vitro assay disclosed in Example 2, anti-TMPRSS6 antibodies MWTx-001, MWTx-002, MWTx-003, hzMWTx-001Var, hzMWTx-002Var, and hzMWTx-003Var increased HAMP promoter activity in a dose-dependent manner (FIGS. 2A-2F), while isotype controls at the same concentrations did not increase HAMP promoter activity.

### Anti-TMPRSS6 Antibodies Having High Affinity for a Target in a Relevant Biological Context

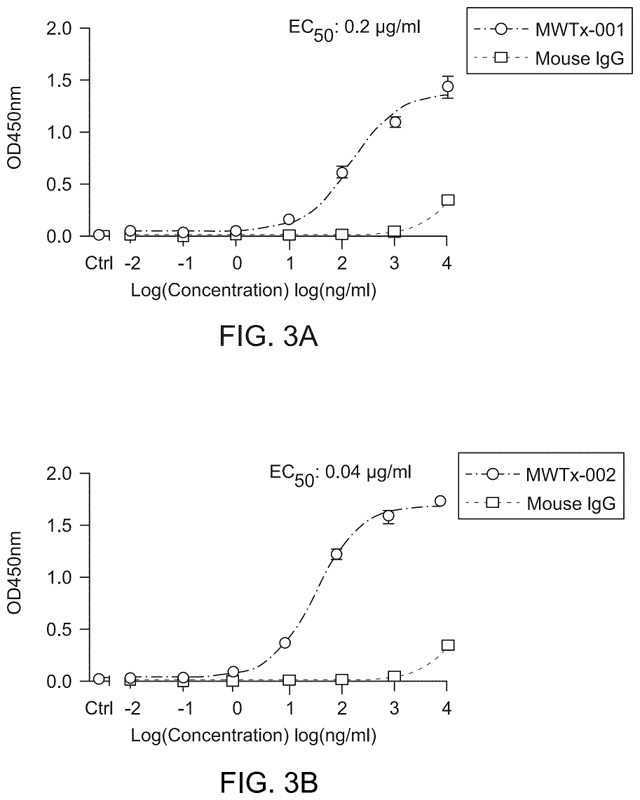

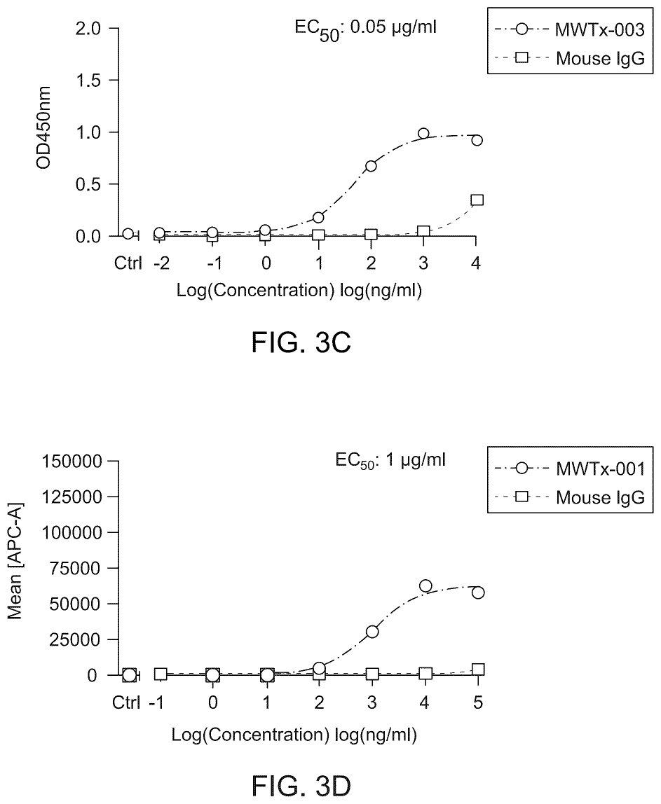

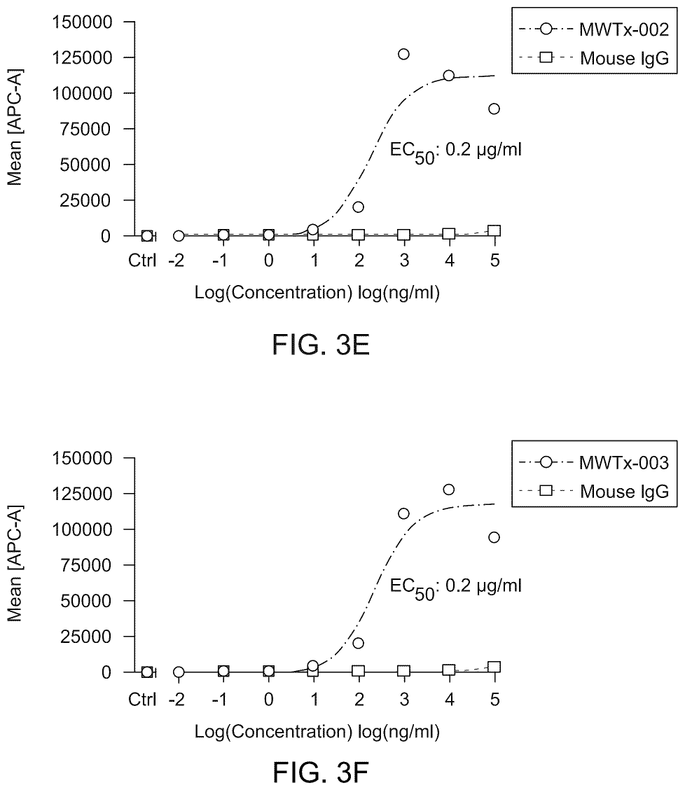

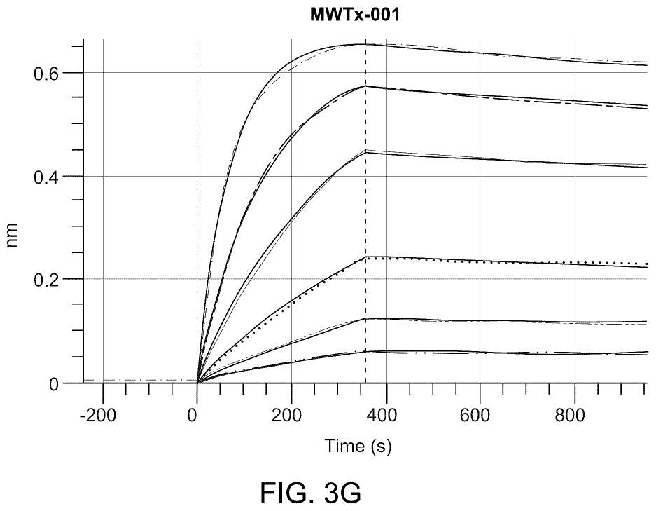

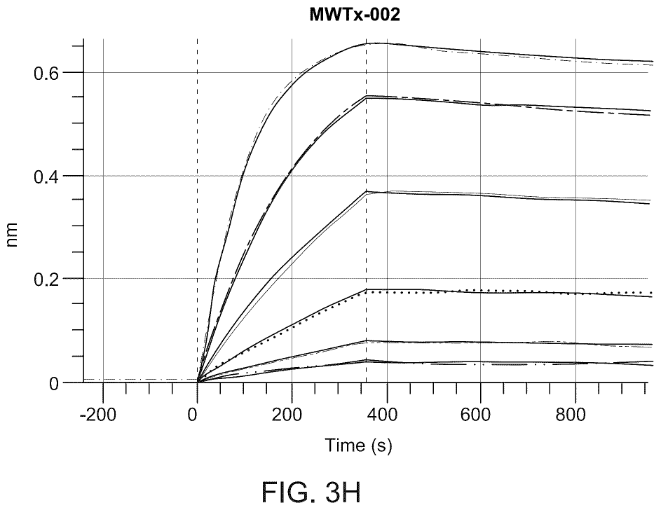

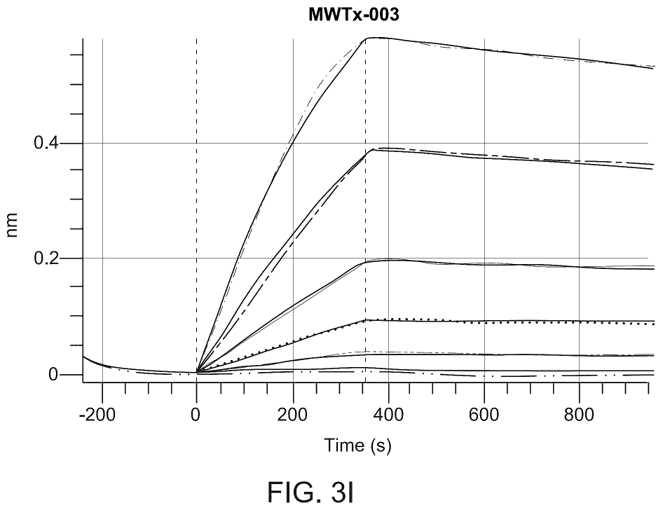

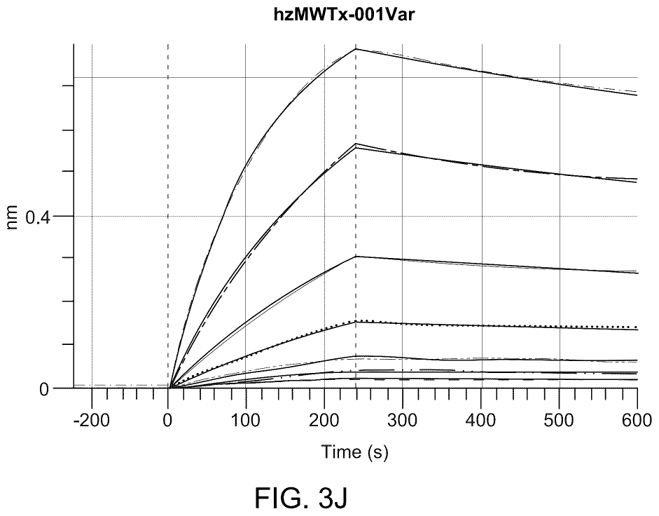

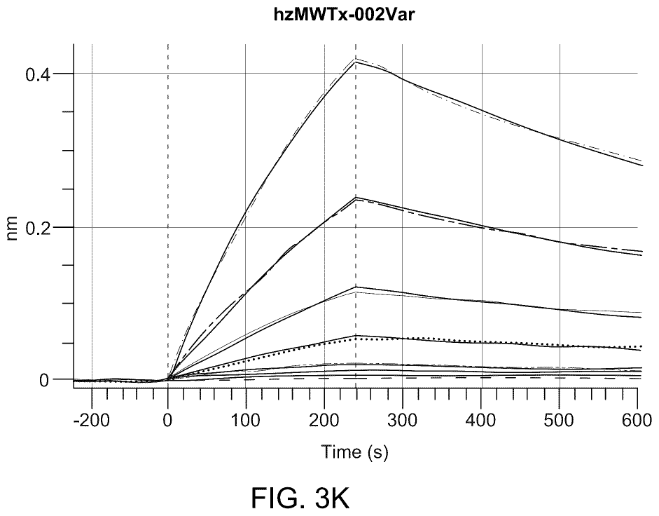

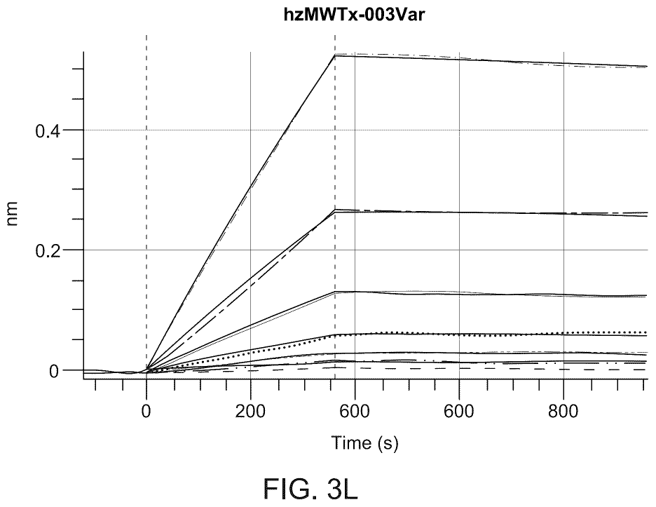

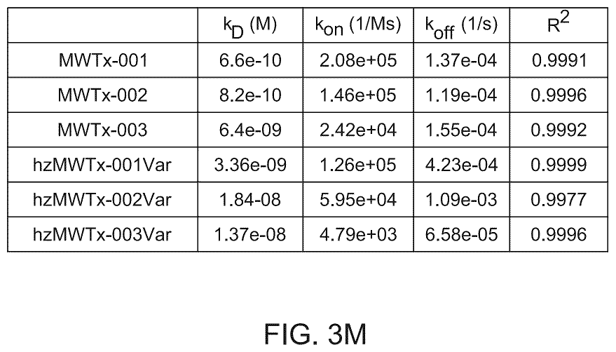

Anti-TMPRSS6 antibodies showed high affinity for a biologically appropriate target, i.e., human TMPRSS6 expressed on the surface of a cell. As demonstrated by exemplary embodiments of affinity measurements using three different methods disclosed in Example 3 and FIG. 3M, monoclonal antibodies MWTx-001, MWTx-002, and MWTx-003, and humanized variants hzMWTx-001Var, hzMWTx-002Var, and hzMWTx-003Var consistently exhibited favorable affinity characteristics for therapeutically effective antibodies or antibody fragments.

### Anti-TMPRSS6 Antibodies Having Cross-Reactivity with Non-Human Targets

It is desirable for therapeutically useful antibodies or antibody fragments to have sufficient cross-reactivity with non-human targets (non-human homologues) from sources that would be relevant for further studies such as preclinical efficacy studies, animal models of disease, toxicology studies, etc., such that the antibodies or antibody fragments should recognize, e.g., a mouse homologue and/or a primate homologue such as from cynomolgus monkey. As demonstrated by exemplary embodiments disclosed in Example 4, MWTx-001, hzMWTx-001Var, MWTx-003, and hzMWTx-003Var showed detectable cross-reactivity with mouse TMPRSS6, while MWTx-001, MWTx-002, MWTx-003, hzMWTx-001Var, hzMWTx-002Var, and hzMWTx-003Var showed detectable cross-reactivity with cynomolgus monkey TMPRSS6.

### Anti-TMPRSS6 Antibodies Specifically Bind TMPRSS6 (Matriptase-2)

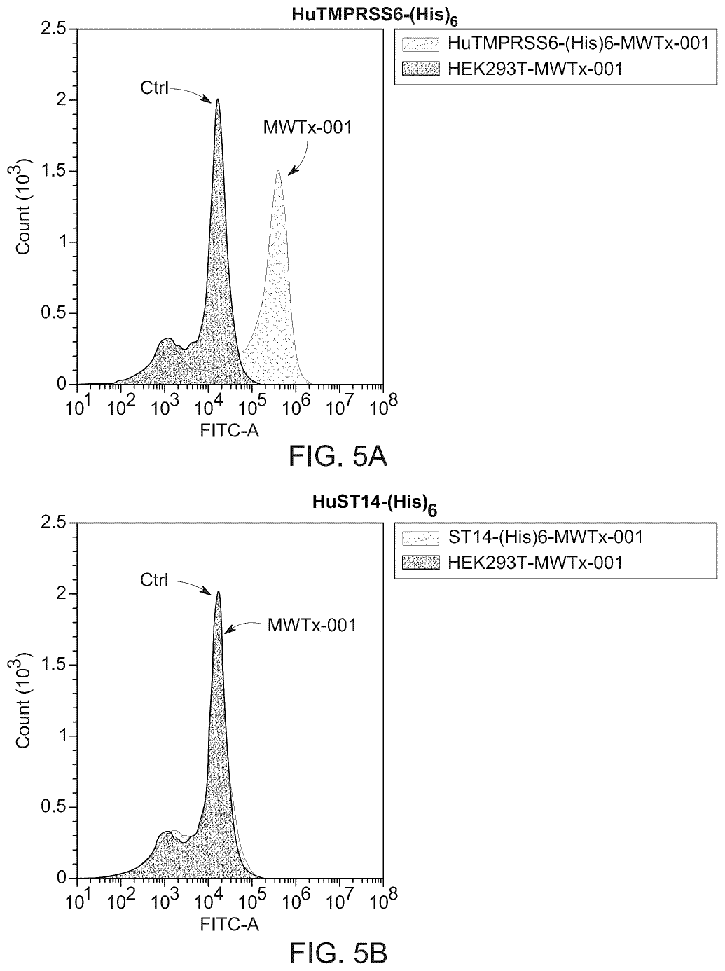

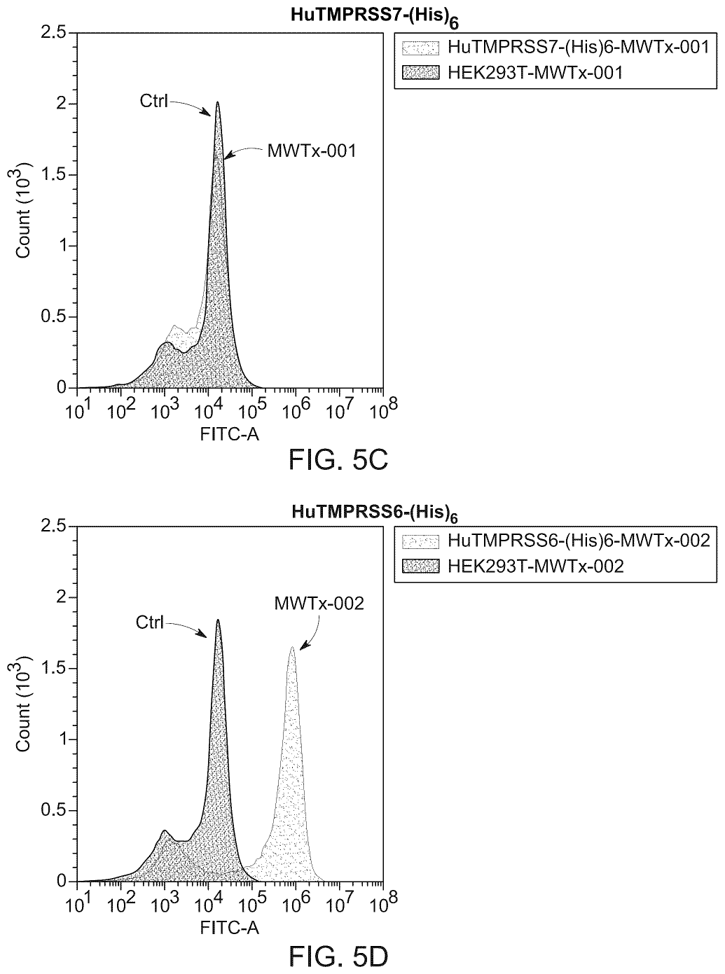

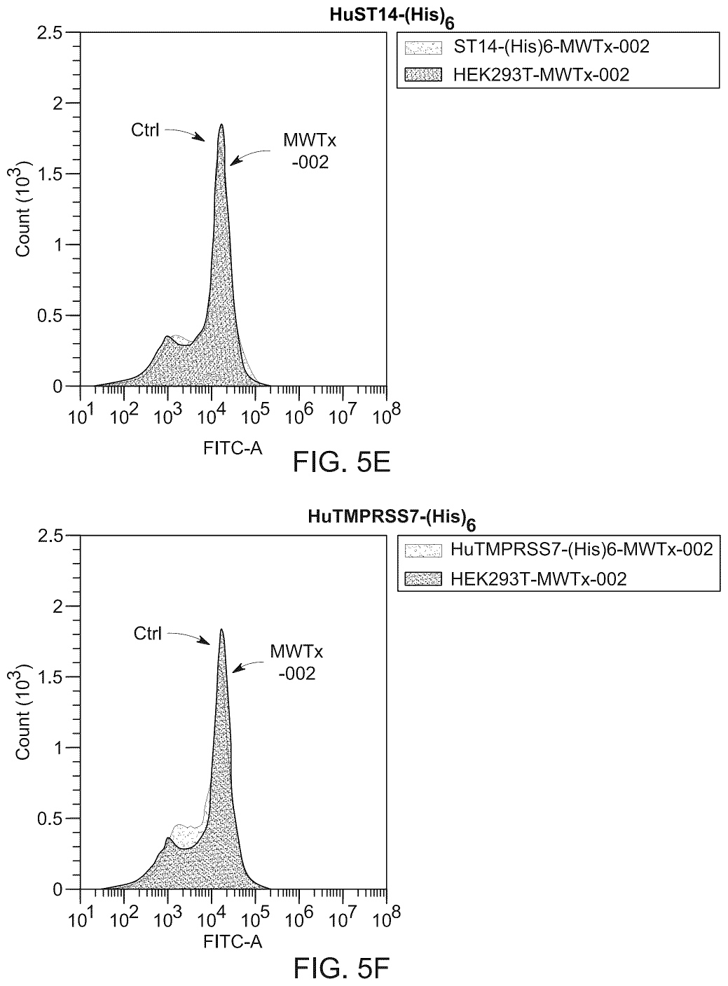

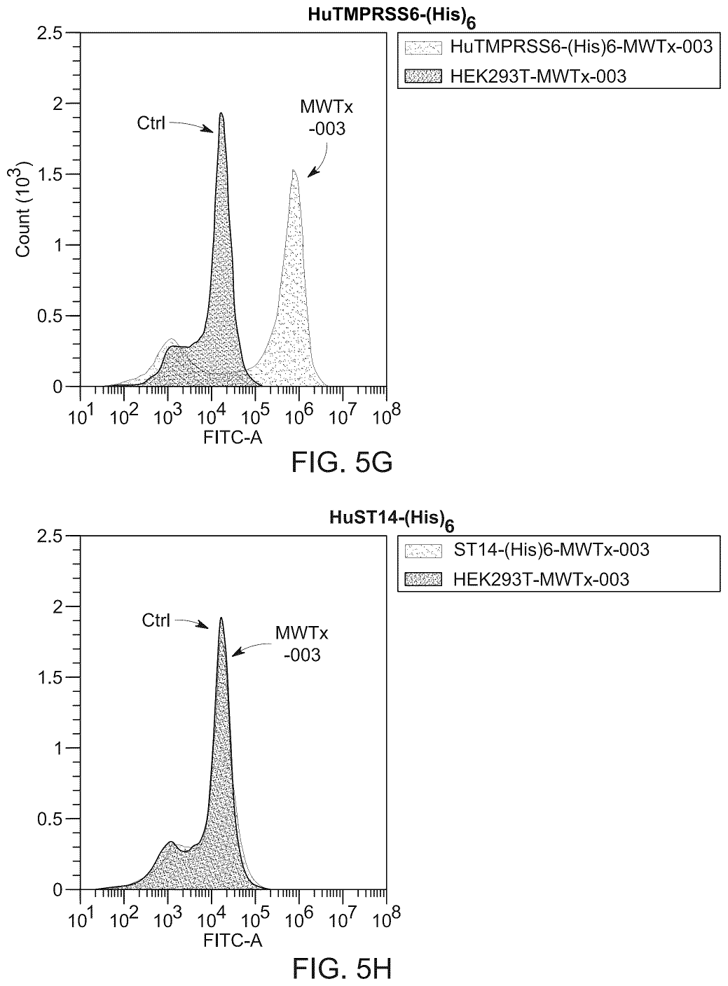

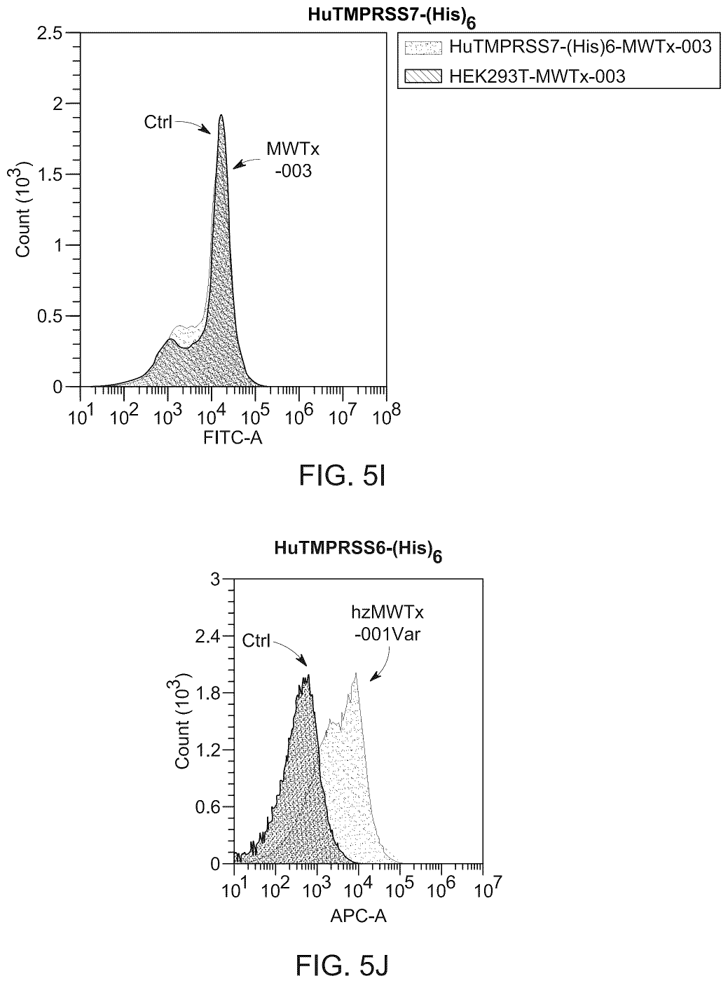

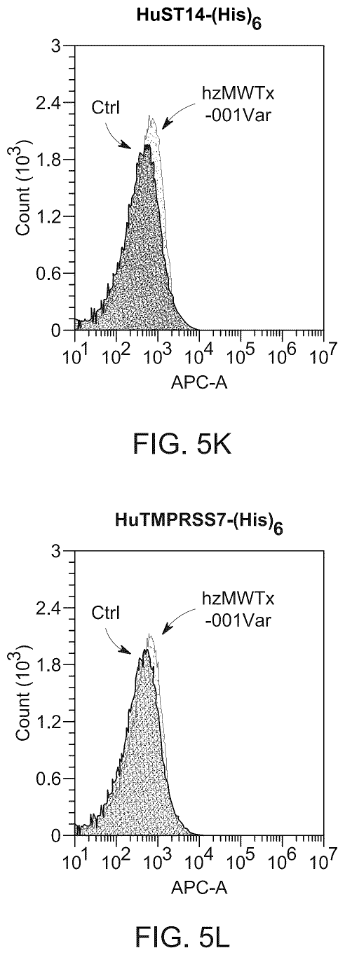

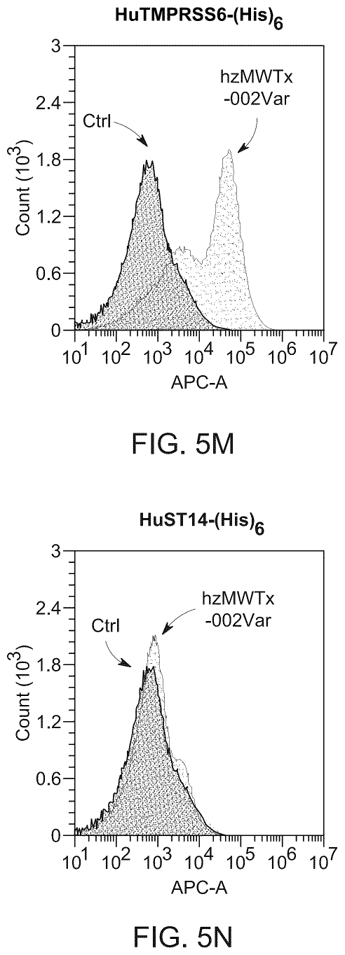

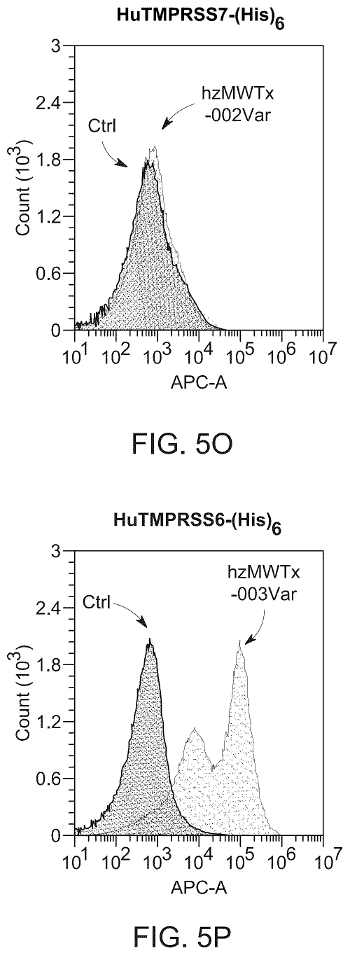

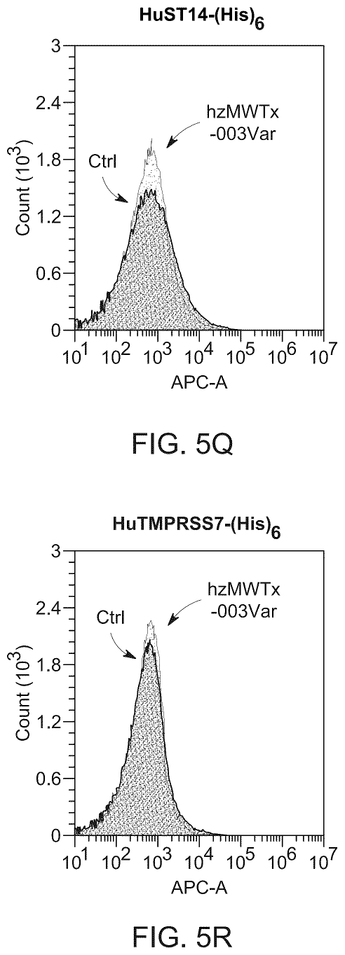

Antibodies with a high level of specific binding to a target protein and low cross-reactivity with homologous proteins in the same organism, are expected to have reduced or no off-target effects. Anti-TMPRSS6 antibodies provided here show high specificity for human TMPRSS6 (matriptase-2), making them suitable for use in targeted compositions and methods. As demonstrated by exemplary embodiments disclosed in Example 5 and illustrated in FIGS. 5A-R, monoclonal antibodies MWTx-001, MWTx-002, and MWTx-003, and their humanized variants hzMWTx-001Var, hzMWTx-002Var, and hzMWTx-003Var show specific binding to human TMPRSS6 (matriptase-2) and did not show detectable cross-reactivity with homologous human matriptases, i.e., these antibodies did not show detectable binding to matriptase-1 (ST14) or matriptase-3 (TMPRSS7).

### Anti-TMPRSS6 Antibodies Having In Vivo Dose-Dependent Effects on Hormones and Symptoms Associated with Iron Overload Disorder

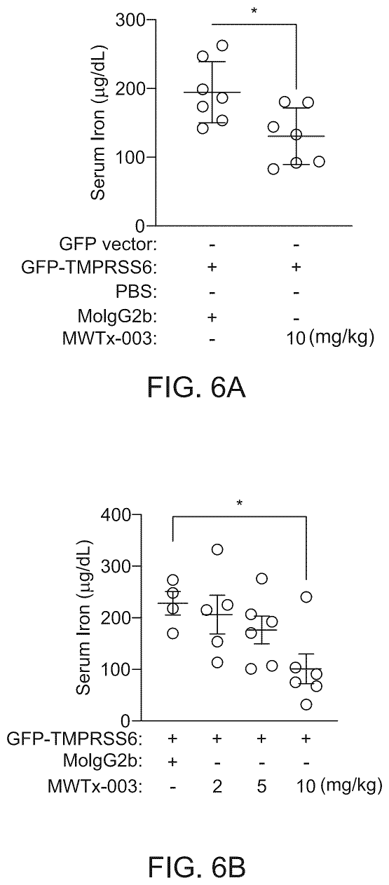

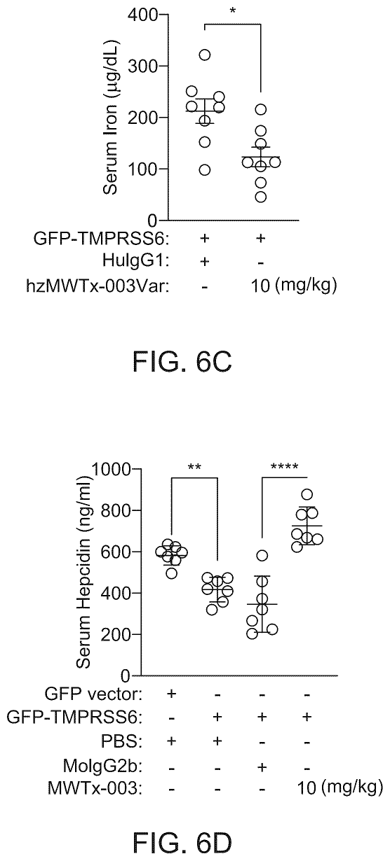

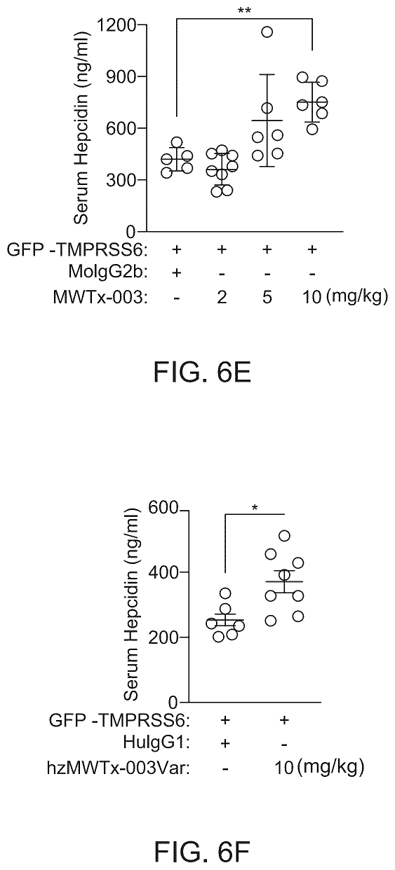

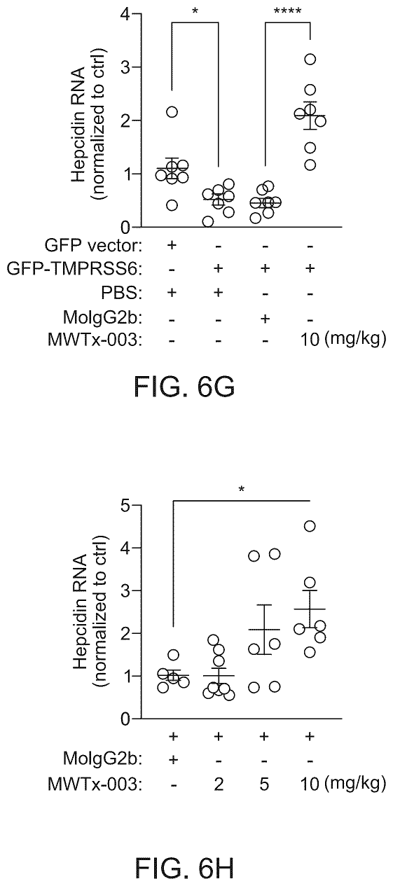

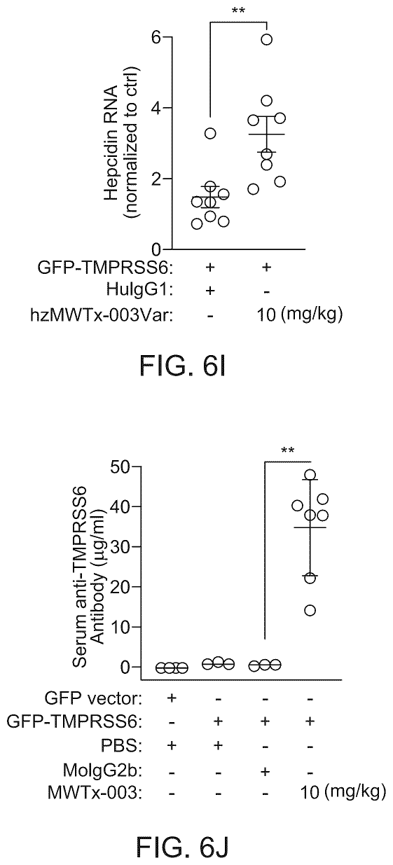

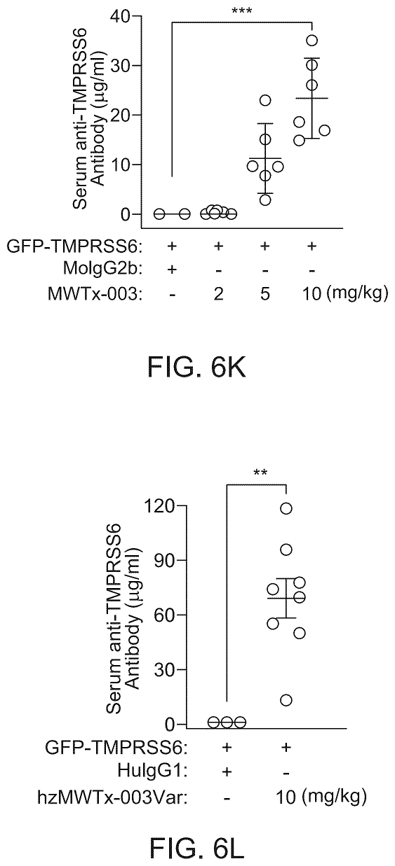

Antibodies that can increase the level of serum hepcidin, a hormone that controls iron absorption and mobilization from iron stores, are expected to reduce, ameliorate, or prevent symptoms of iron overload disorder, in particular to reduce, ameliorate, or prevent symptoms of elevated levels of serum iron. As demonstrated by exemplary embodiments shown in Example 6, administration of anti-TMPRSS6 monoclonal antibody MWTx-003 or humanized variant hzMWTx-003Var to wildtype subjects, i.e., subject that is not known or suspected to have an iron overload, resulted in an increase in serum hepcidin levels (FIGS. 6A-6C), a decrease in serum iron levels (FIGS. 6D to 6F), and an increase in liver hepcidin RNA levels (FIGS. 6G-61) compared with isotype controls. These effects were dose-dependent, which can be interpreted as indicating, without wishing to be bound by a mechanism of action, that the dose-dependent in vivo effects of anti-TMPRSS6 antibodies indicate that a skilled person can determine an effective amount (dosage) for a given subject.

### Anti-TMPRSS6 Antibodies Having In Vivo Efficacy in a β-Thalassemia Disease Model

Antibodies and antibody fragments that can relieve one or more symptoms of an iron overload disorder in vivo when administered to a subject exhibiting an animal model of the disease, i.e., a subject that is known or suspected to have an iron overload disorder, are expected to have therapeutic effectiveness for clinical use. As demonstrated by exemplary embodiments shown in Example 7 using the Th3/+ mouse model of β-thalassemia, administration of the anti-TMPRSS6 monoclonal antibody MWTx-003 resulted in multiple effects including but not limited to reducing liver non-heme iron, increasing serum hepcidin, increasing liver hepcidin RNA, reducing splenomegaly, increasing red blood count (RBC), increasing hematocrit (HCT), reducing red cell distribution width (RDW), and increased production of mature red cells (increased erythropoiesis), compared with isotype controls. Each of these effects can be understood as an amelioration of a symptom of the disorder. Symptoms of the disorder are manifested in multiple biological systems that include but are not limited to effects in the liver (effects on liver non-heme iron, liver hepcidin RNA), in the blood (effects on serum iron levels, circulating hormone levels in particular serum hepcidin levels, RBC, HCT, RDW), spleen size and function (splenomegaly), and erythropoiesis in multiple sites including but not limited to bone marrow and spleen (effects on abundance of different precursor cell types and abundance of mature red cells in erythropoietic sites). Administration of anti-TMPRSS6 antibodies ameliorated multiple symptoms throughout the disease model subject, shifting the measured symptom levels away from levels seen in isotype controls for the disease model (untreated disease) and towards the levels seen in wildtype littermates that represent normal levels in a genetically similar subject that is not known or suspected to have the disease. Without wishing to be bound by a theory or mechanism of action, it is understood that ineffective erythropoiesis is a driving force for abnormal hepcidin suppression leading to increased iron absorption and iron overload, such that a treatment that improves erythroblast differentiation and maturation into red cells should be therapeutically beneficial for treating an iron overload disorder. The present non-limiting exemplary embodiment discloses an anti-TMPRSS6 antibody therapy that increased erythroblast differentiation and maturation into red cells and also decreased iron loading.

### Compositions

Compositions are provided that comprise the anti-TMPRSS6 antibody of the present invention with safe and effective amounts and pharmaceutically acceptable carrier (s) or excipient (s) suitable for the intended use(s) of each composition. Such carriers include but are not limited to: saline, buffer, glucose, water, glycerol, ethanol, excipient, stabilizer, preservative, or combinations thereof. It is understood that the pharmaceutical preparation should match the administration mode.

Anti-TMPRSS6 antibodies disclosed herein can be administered by any suitable means, including but not limited to injection or parenteral infusion. Parenteral infusion can include intramuscular, intravenous, intraarterial, intraperitoneal, subcutaneous administration, or parenteral delivery to the liver. Anti-TMPRSS6 antibodies disclosed herein can be formulated for introduction into hepatic tissue or vasculature for delivery localized to target tissues. Anti-TMPRSS6 antibodies disclosed herein can be administered using a device, or as a depot, or in a sustained-release preparations (e.g., semipermeable matrices of solid hydrophobic polymers containing the antibody, or microcapsules) to allow slow and/or measured and/or localized delivery. Anti-TMPRSS6 antibodies disclosed herein can be formulated and administered using colloidal drug delivery systems (for example, liposomes, albumin microspheres, microemulsions, nano-particles and nanocapsules) or in macroemulsions.

### Methods

Methods are provided for treating a disorder of iron metabolism using an effective amount of an anti-TMPRSS6 antibody disclosed herein. Without wishing to be bound by a particular mechanism of action, methods provided for targeting TMPRSS6 using anti-TMPRSS6 antibodies disclosed herein result in multiple downstream effects, in particular effects on components (molecules, systems, processes) involved in iron metabolism and erythropoiesis. Without wishing to be bound by a particular mechanism of action, methods are provided for treating a disorder of iron metabolism using an effective amount of an anti-TMPRSS6 antibody disclosed herein to modulate the activity of a component involved in iron metabolism. In particular, methods are provided for treating iron overload disorders associated with excess iron accumulation in tissues and organs, including disorders related to or characterized by ineffective erythropoiesis that may include but are not limited to β-thalassemia, in particular non-transfusion dependent thalassemia, MDS (myelodysplastic syndrome), dyserythropoietic anemia, and sideroblastic anemia. Without being limited to a single mechanism of action, methods are provided for treating an iron overload disorders associated with low hepcidin levels, in particular disorders associated with suppressed hepcidin expression, including a disease or state in which abnormal suppression of hepcidin expression is involved, by administering anti-TMPRSS6 antibodies capable of increasing hepcidin expression.

Methods for treating a disorder of iron metabolism as provided herein comprise administering an effective amount of an anti-TMPRSS6 antibody disclosed herein to a subject in need thereof, wherein administration of the effective amount of anti-TMPRSS6 antibody ameliorates at least one biological effect (symptom) associated with the disorder. Methods for treating a disorder of iron metabolism associated with suppressed hepcidin levels are provided wherein administration of an effective amount of an anti-TMPRSS6 antibody disclosed herein to a subject in need thereof, results in at least one of increased hepcidin promoter activity, increased hepcidin transcription, increased hepcidin RNA levels, and increased hepcidin levels, in particular serum hepcidin levels. Methods for treating a subject known or suspected to have an iron overload disorder are provided wherein administration of an effective amount of anti-TMPRSS6 antibody results in one or more biological effects including but not limited to reducing liver non-heme iron, increasing serum hepcidin, increasing liver hepcidin RNA, reducing splenomegaly, increasing red blood count (RBC), increasing hematocrit (HCT), reducing red cell distribution width (RDW), and increased production of mature red cells (increased erythropoiesis). Methods for treating a subject known or suspected to have an iron overload disorder characterized by ineffective erythropoiesis are provided wherein administration of an effective amount of anti-TMPRSS6 antibody results in one or more biological effects including but not limited to reducing liver non-heme iron, increasing serum hepcidin, increasing liver hepcidin RNA, reducing splenomegaly, increasing red blood count (RBC), increasing hematocrit (HCT), reducing red cell distribution width (RDW), and increased production of mature red cells (increased erythropoiesis).

Methods and compositions are provided for treating a disorder of iron metabolism, in particular an iron overload disorder, even more particularly an iron overload disorder characterized by ineffective erythropoiesis, wherein administration of an effective amount of an anti-TMPRSS6 antibody results in treating or ameliorating more than one biological effect or symptom associated with the disorder. Without wishing to be bound by a theory or mechanism of action, it is understood that ineffective erythropoiesis characterized by erythroid precursor apoptosis resulting in few mature red cells produced in the bone marrow, is a driving force for abnormal hepcidin suppression leading to increased iron absorption and iron overload. In accordance with this understanding, a treatment that improves erythroblast differentiation and maturation into red cells should be therapeutically beneficial for treating an iron overload disorder. The effectiveness of anti-TMPRSS6 antibody therapy to increase erythroblast differentiation and maturation into red cells, decrease iron loading, increase hepcidin expression, etc., maximizes the therapeutic benefit of the methods and compositions using anti-TMPRSS6 antibodies disclosed herein.

The following examples are offered to illustrate, but not to limit, the claimed invention.

### EXAMPLES

### Example 1: Antibody Production and Identification of Antibodies that Bind TMPRSS6

The production of novel monoclonal antibodies against TMPRSS6 was carried out under contract by the LakePharma Discovery Immunology group (LakePharma, Inc. San Carlos, CA), utilizing in vivo rodent immunization and hybridoma technology. DNA-based immunization via hydrodynamic gene transfer tail vein injection was performed in B6;SJL mice (The Jackson Laboratories) using a mixture of pLEV113_huTMPRSS6 and pLEV113_moTMPRSS6-TCE plasmid DNA (cloned at LakePharma, Inc). Sufficient plasma titers as determined by fluorescence-activated cell sorting (FACS) were obtained, triggering downstream antibody recovery and screening activities. Electrofusion using a NEPA GENE ECFG21 Super Electro Cell Fusion Generator (Nepa Gene Co., Ltd., Ichikawa-City, Chiba, Japan) was performed with pooled splenocytes from 2 immunized mice and a myeloma fusion partner. Fusion material was plated in a total of ten (10) 384-well plates in hypoxanthine-aminopterin-thymidine medium, which specifically selects for hybridomas over unfused myeloma partner cells. Hybridoma supernatants were initially screened for HuTMPRSS6 reactivity by FACS measurement to detect supernatants that gave a positive staining signal on TMPRSS6-expressing HEK293T cells (a plasmid encoding huTMPRSS6-(His)₆ (SEQ ID NO: 97) was transfected in HEK293T cells, TMPRSS6-expressing HEK293T cells were selected) and negative staining on parentals (HEK293T) on day 10 post-fusion. Hybridoma supernatants giving a positive staining signal on TMPRSS6-expressing HEK293 cells and negative staining on parentals were designated as “hits” for further screening. 192 hits were identified in the primary FACS screen and 143 hits were confirmed in secondary and tertiary FACS screens.

### Example 2. Functional Screening of Anti-TMPRSS6 Antibodies; Identification, Generation, and Sequencing of Monoclonal Anti-TMPRSS6 Antibodies and Humanized Variants

### Hamp-Luciferase Reporter Assay

A hepcidin promoter-luciferase reporter assay was used to measure responses of the HAMP promoter to various anti-TMPRSS6 antibodies (Du, X. et al., 2008. Science 320: 1088-1092; modified to use human HAMP promoter instead of mouse Hamp promoter as originally disclosed). For the HAMP-luciferase report assay, a 2.5 kb HAMP promoter fragment (Reference Genome GRCh38) was spliced upstream from a sequence encoding firefly luciferase. A control construct encoding Renilla luciferase, driven by a thymidine kinase promoter (Promega, E6931) was used as an internal control. These constructs were co-transfected into HepG2 cells (ATCC, HB-8065), together with constructs encoding TMPRSS6. Transfected HepG2 cells expressing TMPRSS6 were pre-treated with various concentrations of purified mAb diluted in starvation medium containing minimum essential medium (MEM, ATCC)+1% heat inactivated fetal bovine serum (FBS, Gibco)+1 mM sodium pyruvate+non-essential amino acids solution (Gibco)+10 mM HEPES (Gibco)+1% Pen/Strep (Gibco) for about 3 hrs before treatment with recombinant hBMP6 (R&D Systems) at a final concentration of 25-60 ng/ml to trigger BMP-SMAD-mediated signaling. Purified mouse IgG (Sigma-Aldrich) or human IgG1 (BioXcell) was used as a control. Upon an overnight treatment of hBMP6, cells were lysed and luciferase substrate were added. Luminescence readings from firefly luciferase and Renilla luciferase were each recorded by measuring total luminescence. Activity was calculated as the ratio of firefly luciferase luminescence to Renilla luciferase luminescence (control). Results for these assays are shown in FIGS. 2A-2F.

### Functional Screening In Vitro

To screen for functionally active hybridomas, the HAMP-luciferase reporter assay described above was used to test all 143 HuTMPRSS6 binding hybridomas (“hits”). Supernatants of ten (10) out of 143 HuTMPRSS6 binding hybridomas increased HAMP promoter activity (data not shown), and were identified as “active clones” to undergo further testing. These ten (10) active clones were tested for cross reactivity against murine target MoTMPRSS6 as described in Example 4 below, and three (3) showed binding towards both HuTMPRSS6 and MoTMPRSS6 as measured by FACS. These three cross-reactive clones were further plated at a density of 1 cell/well in 192 wells of 384-well plates to generate monoclonal hybridoma clones, the resulting subclones that exhibited desired functional activity and cross-reactivity against non-human targets, e.g. murine TMPRSS6 (moTMPRSS6) and/or cynomolgus monkey TMPRSS6 (cynoTMPRSS6) were identified as MWTx-001, MWTx-002, and MWTx-003.

### Sequences of Anti-TMPRSS6 Antibodies MWTx-001, MWTx-002, and MWTx-003

Sequences of MWTx-001, MWTx-002, and MWTx-003 were determined by isolating mRNAs from each hybridoma sample, carrying out reverse transcription polymerase chain reaction (RT-PCR) with unique mouse IgG-specific primer sets to amplify the target variable regions for sequencing. A unique heavy chain and a unique light chain were identified for each anti-TMPRSS6 antibody. The nucleotide sequence of each heavy chain and each light chain was determined. Amino acid sequences encoded by the nucleotide sequences were determined, CDR regions were identified using the Kabat numbering system. Table 1 presents heavy chain and light chain variable region amino acid sequences, and amino acid sequences of identified CDRs (based on Kabat numbering) and heavy chain and light chain variable region nucleotide sequences for each of MWTx-001, MWTx-002, and MWTx-003.

TABLE 1
Sequences of variable regions of anti-TMPRSS6 monoclonal
antibodies MWTx-001, MWTx-002, and MWTx-003

MWTx-001
Heavy chain of MWTx-001:
Protein sequence of the variable region:
QVQLQQPGAELAKPGASVKMSCKASGYTFTSYWITWVKQRPGQDLEWIGNIYPGSGST
YYNEKFKSKATLTVDTSSRTAYMQLSSLTSADSAVYYCAPYDSDYAMDYWGQGTSVT
VSS (SEQ ID NO: 1)

| HC CDR-1 | HC CDR-2 | HC CDR-3 |
|---|---|---|
| GYTFTSYW | IYPGSGST | APYDSDYAMDY |
| (SEQ ID NO: 2) | (SEQ ID NO: 3) | (SEQ ID NO: 4) |

Nucleotide sequence of the variable region:
CAGGTCCAACTGCAGCAGCCTGGGGCTGAGCTTGCGAAGCCTGGGGCTTCAGTGAA
GATGTCCTGCAAGGCTTCTGGCTACACCTTCACCAGCTACTGGATAACCTGGGTGA
AGCAGAGGCCTGGACAAGACCTTGAGTGGATTGGAAATATTTATCCTGGTAGTGGT
AGTACTTACTACAATGAGAAGTTCAAGAGCAAGGCCACACTGACTGTAGACACATC
CTCCAGAACAGCCTACATGCAGCTCAGCAGTCTGACATCTGCGGACTCTGCGGTCT
ATTACTGTGCCCCCTATGATTCCGACTATGCTATGGACTACTGGGGTCAAGGAACCT
CAGTCACCGTCTCCTCA (SEQ ID NO: 5)
Light chain of MWTx-001:
Protein sequence of the variable region:
DIKMTQSPSSMYASLGERVTITCKASQDINNYLSWFQQKPGKSPKTLIYRANRLVDGVP
SRVSGSGSGQDYSLTISSLEYEDVGIYFCLQYDEFPLTFGAGTKLELK (SEQ ID
NO: 6)

| LC CDR-1 | LC CDR-2 | LC CDR-3 |
|---|---|---|
| QDINNY | RAN | LQYDEFPLT |
| (SEQ ID NO: 7) | (SEQ ID NO: 8) | (SEQ ID NO: 9) |

Nucleotide sequence of the variable region
GACATCAAGATGACCCAGTCTCCATCTTCCATGTATGCATCTCTAGGAGAGAGAGT
CACTATCACTTGCAAGGCGAGTCAGGACATTAATAACTATTTAAGCTGGTTCCAGC
AGAAACCAGGGAAATCTCCTAAGACCCTGATCTATCGTGCAAACAGATTGGTAGAT
GGGGTCCCATCAAGGGTCAGTGGCAGTGGATCTGGGCAAGATTATTCTCTCACCAT
CAGCAGCCTGGAGTATGAAGATGTGGGAATTTATTTTTGTCTACAGTATGATGAGTT
TCCTCTCACGTTCGGTGCTGGGACCAAGCTGGAGCTGAAA (SEQ ID NO: 10)
MWTx-002
Heavy chain of MWTx-002:
Protein sequence of the variable region:
EVQLQQSGAELVKPGASVKLSCTASGFNIKDYYIHWVKERTEQGLEWFGRIDPEDGES
EYAPKFQGKATLTADTSSNTAYLQLSSLTSEDTAVYYCTRGDSMMVTYFDYWGQGTT
LTVSSE (SEQ ID NO: 11)

| HC CDR-1 | HC CDR-2 | HC CDR-3 |
|---|---|---|
| GFNIKDYY | IDPEDGES | TRGDSMMVTYFDY |
| (SEQ ID NO: 12) | (SEQ ID NO: 13) | (SEQ ID NO: 14) |

Nucleotide sequence of the variable region:
GAGGTTCAGCTGCAGCAGTCTGGGGCAGAACTTGTGAAGCCAGGGGCCTCAGTCAA
GTTGTCCTGCACAGCCTCTGGCTTCAACATTAAAGACTACTATATACACTGGGTGAA
AGAGAGGACTGAACAGGGCCTGGAGTGGTTTGGAAGGATTGATCCTGAGGATGGT
GAAAGTGAATATGCCCCGAAATTCCAGGGCAAGGCCACTTTAACAGCAGACACATC
CTCCAATACAGCCTACCTGCAGCTCAGCAGCCTGACATCTGAGGACACTGCCGTCT
ATTACTGTACTAGAGGAGACTCTATGATGGTTACCTACTTTGACTACTGGGGCCAA
GGCACCACTCTCACGGTCTCCTCA (SEQ ID NO: 15)
Light chain of MWTx-002:
Protein sequence of the variable region:
DIVMTQSHKFMSTSVGDRVSITCKASQDVSTAVAWYQQKPGQSPKLLIYWAFTRHTGV
PDRFTSTGSGTDYALTISSVQAEDLALYYCQQHYRSPWTFGGGTKLEIK (SEQ ID NO:
16)

| LC CDR-1 | LC CDR-2 | LC CDR-3 |
|---|---|---|
| QDVSTA | WAF | QQHYRSPWT |
| (SEQ ID NO: 17) | (SEQ ID NO: 18) | (SEQ ID NO: 19) |

Nucleotide sequence of the variable region:
GACATTGTGATGACCCAGTCTCACAAATTCATGTCCACATCAGTAGGAGACAGGGT
CAGCATCACCTGCAAGGCCAGTCAGGATGTGAGTACTGCTGTAGCCTGGTATCAAC
AAAAACCAGGGCAATCTCCTAAACTACTGATTTACTGGGCTITCACCCGTCACACT
GGAGTCCCTGATCGCTTCACAAGCACTGGATCTGGGACAGATTATGCTCTCACCAT
CAGCAGTGTGCAGGCTGAAGACCTGGCACTTTATTACTGTCAGCAACATTATCGCA
GTCCGTGGACGTTCGGTGGAGGCACCAAACTGGAAATCAAA (SEQ ID NO: 20)
MWTx-003
Heavy chain of MWTx-003:
Protein sequence of the variable region:
EVQLQQSGAELVKPGASVKLSCTASGFNIEDYYIHWVKERTEQGLEWIGRIDPEDGETT
YAPQFQGKATIIPDTSSNTAYMQLSSLTSEDAAVYYCARSIYLDPMDYWGQGTSVTVSS
(SEQ ID NO: 21)

| HC CDR-1 | HC CDR-2 | HC CDR-3 |
|---|---|---|
| GFNIEDYY | IDPEDGET | ARSIYLDPMDY |
| (SEQ ID NO: 22) | (SEQ ID NO: 23) | (SEQ ID NO: 24) |

Nucleotide sequence of the variable region:
GAAGTTCAGCTGCAGCAGTCTGGGGCAGAACTTGTGAAGCCAGGGGCCTCAGTCAA
GTTGTCCTGCACAGCTTCTGGCTTCAACATTGAAGACTACTATATACACTGGGTGAA
GGAGAGGACTGAACAGGGCCTGGAGTGGATTGGAAGGATTGATCCTGAGGATGGT
GAAACTACATATGCCCCGCAGTTCCAGGGCAAGGCCACTATAATACCAGACACATC
CTCCAACACAGCCTACATGCAGCTCAGCAGCCTGACATCTGAGGACGCTGCCGTCT
ATTACTGTGCTAGATCGATCTACCTTGATCCTATGGACTACTGGGGTCAAGGAACCT
CAGTCACCGTCTCCTCA (SEQ ID NO: 25)
Light chain of MWTx-003:
Protein sequence of the variable region:
DIVMTQSHKFMSTSVGDRVSITCKASQDVTTAVAWYQQKPGQSPKILIYWATTRHTGV
PDRFTGSISGTTYILTISSVQAEDLALYYCQQHYSTPYTFGGGTKLEIK (SEQ ID
NO: 26)

| LC CDR-1 | LC CDR-2 | LC CDR-3 |
|---|---|---|
| QDVTTA | WAT | QQHYSTPYT |
| (SEQ ID NO: 27) | (SEQ ID NO: 28) | (SEQ ID NO: 29) |

Nucleotide sequence of the variable region:
GACATTGTGATGACCCAGTCTCACAAATTCATGTCCACATCAGTAGGAGACAGGGT
CAGCATCACCTGCAAGGCCAGTCAGGATGTGACTACTGCTGTCGCCTGGTATCAAC
AAAAACCAGGACAGTCTCCTAAAATACTGATTTACTGGGCAACCACCCGGCACACT
GGAGTCCCTGATCGCTTCACAGGCAGTATATCTGGGACAACTTATATTCTCACCATC
AGTAGTGTGCAGGCTGAAGACCTGGCACTTTATTACTGTCAGCAACATTATAGCAC
TCCGTACACGTTCGGAGGGGGGACCAAGCTGGAAATAAAA (SEQ ID NO: 30)

### Generation and Screening of Humanized Anti-TMPRSS6 Antibody Variants

Humanization of the parental antibody was performed utilizing CDR grafting onto human antibody frameworks. Homology modeling of the parental antibody's 3-dimensional structure was first performed to establish a structural model of the parental antibody. Amino acid sequences for the variable fragment framework were identified based on the overall sequence identity, matching VH-VL interface positions, similarly classed CDR canonical positions, and removal of potential N-glycosylation sites. Humanized antibodies were designed by creating multiple hybrid sequences that fuse selected parts of the parental antibody sequence with the human framework sequences. The isotypes chosen to format humanized antibody were IgG1 for the heavy chain and IgG1 kappa for the light chain. Using the 3D model, these humanized sequences were methodically analyzed by eye and computer modeling to isolate the sequences that would most likely retain antigen binding. The goal was to maximize the amount of human sequence in the final humanized antibodies while retaining the original antibody specificity. Humanized variants, pairing the humanized VH and VL were then expressed and purified for affinity analysis.

In one round of designing, generating, and testing variants as part of an affinity analysis, four VH variants were generated with the VH-CDRs of the parental antibody MWTX-003 in corresponding positions in four different human IgG1-derived frameworks (SEQ ID NOS: 89-92), and four VL (VK) variants were generated with the VL-CDRs of the parental antibody MWTX-003 in corresponding positions in four different human IgG1 kappa-derived frameworks (SEQ ID NOS: 93-96). A total of sixteen (16) humanized variants representing every combination of the VH and VL (VK) variants were prepared according to a 4VH×4VK matrix, evaluated for antigen binding characteristics (kₒₙ, k_{off}, KD) and found to have KD values in the nanomolar range, from 4.16E−07 (to 1.09E−08.

Variants that showed desired antigen binding affinity were selected for further evaluation and development. In some cases, parental CDR sequences were modified to avoid potential unwanted events such as aspartate isomerization.

To silence antibody effector function, in particular to silence antibody-dependent cellular cytotoxicity (ADCC), critical amino acid residues in the Fc region were identified and mutated (substituted) for all of the humanized antibody variants. Guidance available in the published literature concerning Fc mutations to achieve the goal of abolishing ADCC was used to inform the present mutations, for example removal of the native Fc N-linked glycosylation site (N297A mutation) in hIgG1, or substitutions of leucine at positions 234 and 235 of the lower hinge region in the Fe (LALA double mutation) as described by (Tamm A, Schmidt RE. IgG binding sites on human Fe gamma receptors. Int Rev Immunol. 1997; 16(1-2):57-85. doi: 10.3109/08830189709045703; Jefferis R, Lund J. Interaction sites on human IgG-Fc for FcgammaR: current models. Immunol Lett. 2002 Jun. 3; 82(1-2):57-65. doi: 10.1016/s0165-2478(02)00019-6). In the present variants, the N297A mutation was introduced in the Fc of the hzMWTx-001Var and hzMWTx-002Var antibodies, and the LALA mutation was introduced into the Fc of the hzMWTx-003Var antibody, to achieve the same goal of reducing or silencing ADCC (Table 3, SEQ ID NOs: 73, 77, 81).

After evaluation, humanized anti-TMPRSS6 antibody variants hzMWTx-001Var, hzMWTx-002Var, and hzMWTx-003Var were selected for further testing. Sequences and features of humanized variants are shown in Tables 2 and 3 below.

### Recombinant Production of Humanized Anti-TMPRSS6 Antibody Variants

Expression constructs for humanized anti-TMPRSS6 antibody variants were engineered with internal ribosome entry site (IRES) between LC- and HC-coding DNA sequences, codon optimized by Geneart DNA synthesis and cloned into pcDNA3.4 mammalian expression vector (ThermoFisher). The sequences of DNA inserts were verified by sequencing. For recombinant antibody production, the expression constructs were used for transient transfection using ExpiCHO expression system (ThermoFisher) following manufacturer's instruction. The expressed antibodies were purified by Protein A affinity chromatography. The yield of antibody production from transient transfection ranged from 50 mg to 300 mg per liter, with purity >95% and <1 EU/ml endotoxin level.

### Sequences of Humanized Anti-TMPRSS6 Antibody Variants hzMWTx-001Var, hzMWTx-002Var, and hzMWTx-003Var

Humanized anti-TMPRSS6 antibody variants hzMWTx-001Var, hzMWTx-002Var, and hzMWTx-003Var were selected for further testing. Sequences of the variable region of each variable region are shown in Table 2 below, where identified CDRs are indicated by underlining and changes made in the humanized variant CDR sequences relative to the parental antibody are indicated and discussed.

TABLE 2
Amino acid and nucleotide sequences of variable regions of humanized
anti-TMPRSS6 antibody variants hzMWTx-001Var, hzMWTx-002Var, and
hzMWTx-003Var

hzMWTx-001Var
Heavy chain of hzMWTx-001Var:
Protein sequence of variable region (CDR residues that differ
from parental sequence in bold):
EVQLVQSGAEVKKPGASVKVSCKASGYTFTSYWITWVRQAPGQRLEWIGNIYPGSGST
YYNEKFKSKATITRDTSSRTAYMELSSLRSEDTAVYYCAPYDADYAMDYWGQGTLVT
VSS (SEQ ID NO: 31)

| hzMWTx-001Var | hzMWTx-001Var | hzMWTx-001Var |
|---|---|---|
| HC CDR-1 | HC CDR-2 | HC CDR-3 |
| GYTFTSYW | IYPGSGST | APYDADYAMDY |
| (SEQ ID NO: 32) | (SEQ ID NO: 33) | (SEQ ID NO: 34) |

Comment on underlined sequence DA in hzMWTx-001Var HC CDR-2:
Original sequence in parental antibody is DS here. It was determined that DS is a critical
combination that could result in aspartate isomerization, so S (Serine) at this position was changed
to A (Alanine).
Nucleotide sequence of the variable region:
GAAGTGCAGCTGGTGCAATCTGGCGCCGAAGTGAAGAAACCTGGCGCCTCTGTGAA
GGTGTCCTGCAAGGCTTCCGGCTACACCTTTACCAGCTACTGGATCACCTGGGTCCG
ACAGGCTCCTGGCCAGAGACTGGAATGGATCGGCAACATCTACCCTGGCTCCGGCT
CCACCTACTACAACGAGAAGTTCAAGTCCAAGGCCACAATCACCCGGGACACCTCT
TCCAGAACCGCCTACATGGAACTGTCCAGCCTGAGATCTGAGGACACCGCCGTGTA
CTACTGCGCCCCTTACGACGCCGACTACGCCATGGATTATTGGGGCCAGGGCACCC
TGGTCACCGTGTCCTCT (SEQ ID NO: 35)
Light chain of hzMWTx-001Var:
Protein sequence of variable region (CDR residues that differ from parental sequence in bold):
DIQMTQSPSSLSASVGDRVTITCKASQDISNYLSWFQQKPGKAPKLLIYRANRLVEGVPS
RFSGSGSGTDFTLTISSLQPEDFATYFCLQYDEFPLTFGGGTKVEIK (SEQ ID NO: 36)

| hzMWTx-001Var | hzMWTx-001Var | hzMWTx-001Var |
|---|---|---|
| LC CDR-1 | LC CDR-2 | LC CDR-3 |
| QDISNY | RAN | LQYDEFPLT |
| (SEQ ID NO: 37) | (SEQ ID NO: 38) | (SEQ ID NO: 39) |

Comment on underlined sequence SN in hzMWTx-001Var LC CDR-1:
Original sequence in parental antibody is NN here. It was determined that NN is a critical
combination that could result in asparagine deamidation, so N (Asparagine) at this position
was changed to S (Serine).
Nucleotide sequence of the variable region:
GACATCCAGATGACCCAGTCTCCATCCTCTCTGTCCGCCTCTGTGGGCGACAGAGTG
ACCATCACATGCAAGGCCAGCCAGGACATCTCCAACTACCTGTCCTGGTTCCAGCA
GAAGCCTGGCAAGGCTCCCAAGCTGCTGATCTACAGAGCCAACAGACTGGTGGAA
GGCGTGCCCTCCAGATTCTCCGGATCTGGCTCTGGCACCGACTTTACCCTGACAATC
TCCAGCCTGCAGCCTGAGGACTTCGCTACCTACTTCTGCCTGCAATACGACGAGTTC
CCTCTGACCTTTGGCGGAGGCACCAAGGTGGAAATCAAG (SEQ ID NO: 40)
hzMWTx-002Var
Heavy chain of hzMWTx-002Var:
Protein sequence of variable region (CDR residues that differ from parental sequence in bold):
EVQLVQSGAEVKKPGASVKVSCKASGFNIKDYYIHWVRQATGQGLEWMGRIDPEDAE
SEYAPKFQGRVTITADTSTDTAYMELSSLRSEDTAVYYCTRGDSMMVTYFDYWGQGT
LVTVSS (SEQ ID NO: 41)

| hzMWTx-002Var | hzMWTx-002Var | hzMWTx-002Var |
|---|---|---|
| HC CDR-1 | HC CDR-2 | HC CDR-3 |
| GFNIKDYY | IDPEDAES | TRGDSMMVTYFDY |
| (SEQ ID NO: 42) | (SEQ ID NO: 43) | (SEQ ID NO: 44) |

Comment on underlined sequence DA in hzMWTx-002Var HC CDR-2:
Original sequence in parental antibody is DG here. It was determined that DG is a critical
combination that could likely result in aspartate isomerization, so G (Glycine) at this position
was changed to A (Alanine).
Nucleotide sequence of the variable region:
GAAGTGCAGCTGGTGCAATCTGGCGCCGAAGTGAAGAAACCTGGCGCCTCTGTGAA
GGTGTCCTGCAAGGCCTCTGGCTTCAACATCAAGGACTACTACATCCACTGGGTCC
GACAGGCTACCGGACAGGGACTTGAGTGGATGGGCAGAATCGACCCTGAGGACGC
CGAGTCTGAGTACGCCCCTAAGTTTCAGGGCAGAGTGACCATCACCGCCGACACCT
CTACCGACACCGCCTACATGGAACTGTCCAGCCTGAGATCTGAGGACACCGCCGTG
TACTACTGCACCAGAGGCGACTCCATGATGGTTACCTACTTCGACTACTGGGGCCA
GGGCACCCTGGTCACAGTTTCTTCC (SEQ ID NO: 45)
Light chain of hzMWTx-002Var:
Protein sequence of variable region:
DIQMTQSPSSLSASVGDRVTITCKASQDVSTAVAWYQQKPGKAPKLLIYWAFTRHTGV
PSRFSGSGSGTDYALTISSLQPEDFATYYCQQHYRSPWTFGGGTKVEIK (SEQ ID NO:
46)

| hzMWTx-002Var | hzMWTx-002Var | hzMWTx-002Var |
|---|---|---|
| LC CDR-1 | LC CDR-2 | LC CDR-3 |
| QDVSTA | WAF | QQHYRSPWT |
| (SEQ ID NO: 47) | (SEQ ID NO: 48) | (SEQ ID NO: 49) |

Nucleotide sequence of the variable region:
GACATCCAGATGACCCAGTCTCCATCCTCTCTGTCCGCCTCTGTGGGCGACAGAGTG
ACCATCACATGCAAGGCCTCTCAGGACGTGTCCACCGCCGTTGCTTGGTATCAGCA
GAAGCCTGGCAAGGCCCCTAAGCTGCTGATCTACTGGGCCTTCACCAGACACACCG
GCGTGCCCTCTAGGTTCTCCGGCTCTGGCTCTGGCACCGATTACGCTCTGACAATCT
CCAGCCTGCAGCCTGAGGACTTCGCCACCTACTACTGCCAGCAGCACTACAGAAGC
CCCTGGACATTTGGCGGAGGCACCAAGGTGGAAATCAAG (SEQ ID NO: 50)
hzMWTx-003Var
Heavy chain of hzMWTx-003Var:
Protein sequence of variable region (CDRs indicated by underlining; CDR residues that differ
from parental CDR sequence indicated in bold):
QVQLVQSGAEVKKPGASVKVSCKASGFNIEDYYMHWVRQAPGQRLEWMGRIDPEDA
ETTYSPKFQGRVTIIPDTSANTAYMELSSLRSEDTAVYYCARSIYLDPMDYWGQGTLVT
VSS (SEQ ID NO: 51)

| hzMWTx-03Var | hzMWTx-003Var | hzMWTx-003Var |
|---|---|---|
| HC CDR-1 | HC CDR-2 | HC CDR-3 |
| GFNIEDYY | IDPEDAET | ARSIYLDPMDY |
| (SEQ ID NO: 52) | (SEQ ID NO: 53) | (SEQ ID NO: 54) |

Comment on underlined sequence DA in hzMWTx-003Var HC CDR-2:
Original sequence in parental antibody is DG here. It was determined that DG is a critical
combination that could likely result in aspartate isomerization, so G (Glycine) at this position
was changed to A (Alanine).
Nucleotide sequence of the variable region:
CAGGTGCAGCTGGTGCAGTCTGGCGCCGAAGTGAAAAAGCCTGGCGCCTCTGTGAA
GGTGTCCTGCAAGGCCTCTGGCTTCAACATCGAGGACTACTACATGCACTGGGTCC
GACAGGCCCCTGGCCAGAGATTGGAATGGATGGGCAGAATCGACCCCGAGGACGC
CGAGACAACCTACTCTCCTAAGTTCCAGGGCCGCGTGACAATCATCCCTGACACCT
CTGCCAACACCGCCTACATGGAACTGTCCAGCCTGAGATCTGAGGACACCGCCGTG
TACTACTGCGCCCGGTCTATCTACCTGGACCCTATGGACTATTGGGGCCAGGGCAC
CCTGGTCACAGTGTCCTCT (SEQ ID NO: 55)
Light chain of hzMWTx-003Var:
Protein sequence of variable region:
DIQMTQSPKSLSASVGDRVTITCRASQDVTTALAWYQQKPGQSPKLLIYWATTRHSGV
PSRFSGSGSGTDFTLTISSLQPEDFATYYCQQHSTPYTFGQGTKLEIK (SEQ ID NO: 56)

| hzMWTx-003Var | hzMWTx-003Var | hzMWTx-003Var |
|---|---|---|
| LC CDR-1 | LC CDR-2 | LC CDR-3 |
| QDVTTA | WAT | QQHYSTPYT |
| (SEQ ID NO: 57) | (SEQ ID NO: 58) | (SEQ ID NO: 59) |

Nucleotide sequence of the variable region:
GACATCCAGATGACCCAGTCTCCAAAGTCTCTGTCCGCCTCCGTGGGCGACAGAGT
GACCATCACCTGTAGAGCCTCTCAGGACGTGACCACCGCTCTGGCTTGGTATCAGC
AGAAGCCTGGCCAGTCTCCTAAGCTGCTGATCTACTGGGCCACCACCAGACACTCT
GGCGTGCCCTCTAGATTCTCCGGCTCTGGCTCTGGCACCGACTTTACCCTGACAATC
TCCAGCCTGCAGCCTGAGGACTTCGCCACCTACTACTGCCAGCAGCACTACAGCAC
CCCTTACACCTTTGGCCAGGGCACCAAGCTGGAAATCAAG (SEQ ID NO: 60)

Table 3 shows complete heavy chain and light chain protein and nucleotide sequences of anti-TMPRSS6 monoclonal antibodies MWTx-001, MWTx-002, and MWTx-003, and humanized anti-TMPRSS6 antibody variants hzMWTx-001Var, hzMWTx-002Var, and hzMWTx-003Var. Heavy chain protein sequences of humanized anti-TMPRSS6 antibody variants hzMWTx-OOlVar, hzMWTx-002Var, and hzMWTx-003Var show the location of mutations (changes) introduced to reduce ADCC as described above.

TABLE 3
Heavy chain and light chain sequences of anti-TMPRSS6 monoclonal
antibodies MWTx-001, MWTx-002, and MWTx-003, and humanized anti-
TMPRSS6 antibody variants hzMWTx-001Var, hzMWTx-002Var, and hzMWTx-003Var
Anti-TMPRSS6 monoclonal antibodies
MWTx-001
Heavy chain of MWTx-001:
Protein sequence (Constant region indicated by italics):
QVQLQQPGAELAKPGASVKMSCKASGYTFTSYWITWVKQRPGQDLEWIGNIYPGSGST
YYNEKFKSKATLTVDTSSRTAYMQLSSLTSADSAVYYCAPYDSDYAMDYWGQGTSVT
VSSAKTTAPSVYPLAPVCGDTTGSSVTLGCLVKGYFPEPVTLTWNSGSLSSGVHTFPAVLQSD
LYTLSSSVTVTSSTWPSQSITCNVAHPASSTKVDKKIEPRGPTIKPCPPCKCPAPNLLGGPSVFI
FPPKIKDVLMISLSPIVTCVVVDVSEDDPDVQISWFVNNVEVHTAQTQTHREDYNSTLRVVSA
LPIQHQDWMSGKEFKCKVNNKDLPAPIERTISKPKGSVRAPQVYVLPPPEEEMTKKQVTLTC
MVTDFMPEDIYVEWTNNGKTELNYKNTEPVLDSDGSYFMYSKLRVEKKNWVERNSYSCSVV
HEGLHNHHTTKSFSRTPG (SEQ ID NO: 61)
Nucleotide sequence:
CAGGTCCAACTGCAGCAGCCTGGGGCTGAGCTTGCGAAGCCTGGGGCTTCAGTGAAGA
TGTCCTGCAAGGCTTCTGGCTACACCTTCACCAGCTACTGGATAACCTGGGTGAAGCAG
AGGCCTGGACAAGACCTTGAGTGGATTGGAAATATTTATCCTGGTAGTGGTAGTACTTA
CTACAATGAGAAGTTCAAGAGCAAGGCCACACTGACTGTAGACACATCCTCCAGAACA
GCCTACATGCAGCTCAGCAGTCTGACATCTGCGGACTCTGCGGTCTATTACTGTGCCCCC
TATGATTCCGACTATGCTATGGACTACTGGGGTCAAGGAACCTCAGTCACCGTCTCCTC
AGCTAAAACAACAGCCCCATCGGTCTATCCACTGGCCCCTGTGTGTGGAGATACAACTG
GCTCCTCGGTGACTCTAGGATGCCTGGTCAAGGGTTATTTCCCTGAGCCAGTGACCTTG
ACCTGGAACTCTGGTTCCCTGTCCAGTGGTGTGCACACCTTCCCAGCTGTCCTGCAGTCT
GACCTCTACACCCTCAGCTCAAGCGTGACTGTAACCAGCTCGACCTGGCCCAGCCAGTC
CATCACCTGCAATGTGGCCCACCCGGCAAGCAGCACCAAGGTGGACAAGAAAATTGAG
CCCAGAGGGCCCACAATCAAGCCCTGTCCTCCATGCAAATGCCCAGCACCTAACCTCTT
GGGTGGACCATCCGTCTTCATCTTCCCTCCAAAGATCAAGGATGTACTCATGATCTCCCT
GAGCCCCATAGTCACATGTGTAGTCGTTGATGTGAGCGAGGATGACCCAGATGTCCAGA
TCAGCTGGTTTGTGAACAACGTGGAAGTGCACACTGCTCAGACACAGACGCATAGAGA
GGATTACAACAGTACTCTCCGGGTTGTCAGTGCCCTCCCCATCCAGCACCAGGACTGGA
TGAGTGGCAAGGAGTTCAAATGCAAGGTCAACAACAAAGACCTCCCAGCGCCCATCGA
GAGAACCATCTCAAAACCCAAAGGGTCAGTAAGAGCTCCACAGGTATATGTCTTGCCTC
CACCAGAAGAGGAGATGACTAAGAAACAGGTCACTCTGACCTGCATGGTCACAGACTT
CATGCCTGAAGACATTTACGTGGAGTGGACCAACAACGGGAAAACAGAGCTAAACTAC
AAGAACACTGAACCAGTCCTGGACTCTGATGGTTCTTACTTCATGTACAGCAAGCTGAG
AGTGGAGAAGAAGAACTGGGTGGAGAGAAATAGCTACTCCTGTTCAGTGGTCCACGAG
GGTCTGCACAATCACCACACGACTAAGAGCTTCTCCCGGACTCCGGGTTAGTAA (SEQ
ID NO: 62)
Light chain of MWTx-001:
Protein sequence (Constant region indicated by italics):
DIKMTQSPSSMYASLGERVTITCKASQDINNYLSWFQQKPGKSPKTLIYRANRLVDGVP
SRVSGSGSGQDYSLTISSLEYEDVGIYFCLQYDEFPLTFGAGTKLELKRADAAPTVSIFPPS
SEQLTSGGASVVCFLNNFYPKDINVKWKIDGSERQNGVLNSWTDQDSKDSTYSMSSTLTLTK
DEYERHNSYTCEATHKTSTSPIVKSFNRNEC (SEQ ID NO: 63)
Nucleotide sequence:
GACATCAAGATGACCCAGTCTCCATCTTCCATGTATGCATCTCTAGGAGAGAGAGTCAC
TATCACTTGCAAGGCGAGTCAGGACATTAATAACTATTTAAGCTGGTTCCAGCAGAAAC
CAGGGAAATCTCCTAAGACCCTGATCTATCGTGCAAACAGATTGGTAGATGGGGTCCCA
TCAAGGGTCAGTGGCAGTGGATCTGGGCAAGATTATTCTCTCACCATCAGCAGCCTGGA
GTATGAAGATGTGGGAATTTATTTTTGTCTACAGTATGATGAGTTTCCTCTCACGTTCGG
TGCTGGGACCAAGCTGGAGCTGAAAAGAGCTGACGCCGCTCCTACCGTGTCCATCTTTC
CACCTAGCAGCGAGCAGCTGACAAGCGGCGGAGCCAGCGTCGTGTGCTTCCTGAACAA
CTTCTACCCCAAGGACATCAACGTGAAGTGGAAGATCGACGGCAGCGAGAGACAGAAC
GGCGTGCTGAATAGCTGGACCGACCAGGACAGCAAGGACTCCACCTACAGCATGTCCA
GCACACTGACCCTGACCAAGGACGAGTACGAGCGGCACAACAGCTACACATGCGAGGC
CACACACAAGACCAGCACAAGCCCCATCGTGAAGTCCTTCAACCGGAACGAGTGC (SEQ
ID NO: 64)
MWTx-002
Heavy chain of MWTx-002:
Protein sequence (Constant region indicated by italics):
EVQLQQSGAELVKPGASVKLSCTASGFNIKDYYIHWVKERTEQGLEWFGRIDPEDGESE
YAPKFQGKATLTADTSSNTAYLQLSSLTSEDTAVYYCTRGDSMMVTYFDYWGQGTTL
TVSSKTTPPSVYPLAPGCGDTTGSSVTLGCLVKGFPESVTVTWNSGSLSSSVHTFPALLQSGL
YTMSSSVTVPSSTWPSQTVTCSVAHPASSTTVDKKLEPSGPISTINPCPPCKECHKCPAPNLEG
GPSVFIFPPNIKDVLMISLTPKVTCVVVDVSEDDPDVQISWFVNNVEVHTAQTQTHREDYNST
IRVVSTLPIQHQDWMSGKEFKCKVNNKDLPSPIERTISKIKGLVRAPQVYILPPPAEQLSRKDV
SLTCLVVGFNPGDISVEWTSNGHTEENYKDTAPVLDSDGSYFIYSKLNMKTSKWEKTDSFSCN
VRHEGLKNYYLKKTISRSPGK (SEQ ID NO: 65)
Nucleotide sequence:
GAGGTTCAGCTGCAGCAGTCTGGGGCAGAACTTGTGAAGCCAGGGGCCTCAGTCAAGT
TGTCCTGCACAGCCTCTGGCTTCAACATTAAAGACTACTATATACACTGGGTGAAAGAG
AGGACTGAACAGGGCCTGGAGTGGTTTGGAAGGATTGATCCTGAGGATGGTGAAAGTG
AATATGCCCCGAAATTCCAGGGCAAGGCCACTTTAACAGCAGACACATCCTCCAATACA
GCCTACCTGCAGCTCAGCAGCCTGACATCTGAGGACACTGCCGTCTATTACTGTACTAG
AGGAGACTCTATGATGGTTACCTACTTTGACTACTGGGGCCAAGGCACCACTCTCACGG
TCTCCTCAAAGACCACACCTCCTAGCGTGTACCCTCTGGCTCCTGGCTGTGGCGATACA
ACAGGCAGCTCTGTGACACTGGGCTGCCTGGTCAAGGGCTACTTTCCTGAGAGCGTGAC
AGTGACCTGGAACAGCGGCAGCCTGTCTAGCAGCGTGCACACCTTTCCAGCTCTGCTCC
AGAGCGGCCTGTACACCATGTCCTCTAGTGTGACCGTGCCTAGCAGCACCTGGCCTAGC
CAGACAGTGACATGTAGCGTGGCCCATCCTGCCAGCAGCACAACCGTGGACAAGAAGC
TGGAACCTAGCGGCCCCATCAGCACCATCAATCCCTGTCCTCCATGCAAAGAATGCCAC
AAGTGCCCCGCTCCTAACCTGGAAGGTGGCCCAAGCGTGTTCATCTTCCCACCTAACAT
CAAGGACGTGCTGATGATCAGCCTGACACCTAAAGTGACCTGCGTGGTGGTGGACGTGT
CCGAGGATGATCCCGATGTGCAGATCAGTTGGTTCGTGAACAACGTGGAAGTGCACAC
AGCCCAGACACAGACCCACAGAGAGGACTACAATAGCACCATTCGCGTGGTGTCCACA
CTGCCTATCCAGCACCAGGATTGGATGAGCGGCAAAGAGTTCAAGTGCAAAGTGAACA
ACAAGGACCTGCCTTCTCCAATCGAGCGGACCATCAGCAAGATCAAGGGACTCGTCAG
AGCCCCTCAGGTGTACATCTTGCCTCCACCAGCCGAGCAGCTGAGCAGAAAGGATGTGT
CCGTGACCTGTCTGGTCGTGGGCTTCAACCCTGGCGACATCAGCGTGGAATGGACCAGC
AATGGCCACACCGAGGAAAACTACAAGGACACAGCCCCTGTGCTGGACAGCGACGGCA
GCTACTTCATCTACAGCAAGCTGAACATGAAGACCAGCAAGTGGGAGAAAACCGACAG
CTTCTCCTGCAACGTGCGGCACGAGGGCCTGAAGAACTACTACCTGAAGAAAACCATCT
CTCGGAGCCCCGGCAAG (SEQ ID NO: 66)
Light chain of MWTx-002:
Protein sequence (Constant region indicated by italics):
DIVMTQSHKFMSTSVGDRVSITCKASQDVSTAVAWYQQKPGQSPKLLIYWAFTRHTGV
PDRFTSTGSGTDYALTISSVQAEDLALYYCQQHYRSPWTFGGGTKLEIKRADAAPTVSIF
PPSSEQLTSGGASVVCFLNNFYPKDINVKWKIDGSERQNGVLNSWTDQDSKDSTYSMSSTLTL
TKDEYERHNSYTCEATHKTSTSPIVKSFNRNEC (SEQ ID NO: 67)
Nucleotide sequence:
GACATTGTGATGACCCAGTCTCACAAATTCATGTCCACATCAGTAGGAGACAGGGTCAG
CATCACCTGCAAGGCCAGTCAGGATGTGAGTACTGCTGTAGCCTGGTATCAACAAAAAC
CAGGGCAATCTCCTAAACTACTGATTTACTGGGCTTTCACCCGTCACACTGGAGTCCCTG
ATCGCTTCACAAGCACTGGATCTGGGACAGATTATGCTCTCACCATCAGCAGTGTGCAG
GCTGAAGACCTGGCACTTTATTACTGTCAGCAACATTATCGCAGTCCGTGGACGTTCGG
TGGAGGCACCAAACTGGAAATCAAAAGAGCTGACGCCGCTCCTACCGTGTCCATCTTTC
CACCTAGCAGCGAGCAGCTGACAAGCGGCGGAGCCAGCGTCGTGTGCTTCCTGAACAA
CTTCTACCCCAAGGACATCAACGTGAAGTGGAAGATCGACGGCAGCGAGAGACAGAAC
GGCGTGCTGAATAGCTGGACCGACCAGGACAGCAAGGACTCCACCTACAGCATGTCCA
GCACACTGACCCTGACCAAGGACGAGTACGAGCGGCACAACAGCTACACATGCGAGGC
CACACACAAGACCAGCACAAGCCCCATCGTGAAGTCCTTCAACCGGAACGAGTGC
(SEQ ID NO: 68)
MWTx-003
Heavy chain of MWTx-003:
Protein sequence (Constant region indicated by italics):
EVQLQQSGAELVKPGASVKLSCTASGFNIEDYYIHWVKERTEQGLEWIGRIDPEDGETT
YAPQFQGKATIIPDTSSNTAYMQLSSLTSEDAAVYYCARSIYLDPMDYWGQGTSVTVSS
KTTPPSVYPLAPGCGDTTGSSVTLGCLVKGYFPESVTVTWNSGSLSSSVHTFPALLQSGLYTMS
SSVTVPSSTWPSQTVTCSVAHPASSTTVDKKLEPSGPISTINPCPPCKECHKCPAPNLEGGPSV
FIFPPNIKDVLMISLTPKVTCVVVDVSEDDPDVQISWFVNNVEVHTAQTQTHREDYNSTIRVV
STLPIQHQDWMSGKEFKCKVNNKDLPSPIERTISKIKGLVRAPQVYILPPPAEQLSRKDVSLTC
LVVGFNPGDISVEWTSNGHTEENYKDTAPVLDSDGSYFIYSKLNMKTSKWEKTDSFSCNVRH
EGLKNYYLKKTISRSPGK (SEQ ID NO: 69)
Nucleotide sequence:
GAGGTTCAGCTGCAGCAGTCTGGCGCCGAGCTTGTGAAACCTOGCGCCTCTGTGAAGCT
GAGCTGTACCGCCAGCGGCTTCAACATCGAGGACTACTACATCCACTGGGTCAAAGAGC
GGACCGAGCAGGGACTCGAGTGGATCGGAAGAATCGACCCCGAGGACGGCGAGACAA
CATACGCCCCTCAGTTTCAGGGCAAAGCCACAATCATCCCCGACACCAGCAGCAACACC
GCCTACATGCAACTGAGCAGCCTGACCTCTGAAGATGCCGCCGTGTACTACTGCGCCCG
GTCCATCTATCTGGACCCCATGGATTATTGGGGCCAGGGCACAAGCGTGACCGTGTCCT
CTAAGACCACACCTCCTAGCGTGTACCCTCTGGCTCCTGGCTGTGGCGATACAACAGGC
AGCTCTGTGACACTGGGCTGCCTGGTCAAGGGCTACTTTCCTGAGAGCGTGACAGTGAC
CTGGAACAGCGGCAGCCTGTCTAGCAGCGTGCACACCTTTCCAGCTCTGCTCCAGAGCG
GCCTGTACACCATGTCCTCTAGTGTGACCGTGCCTAGCAGCACCTGGCCTAGCCAGACA
GTGACATGTAGCGTGGCCCATCCTGCCAGCAGCACAACCGTGGACAAGAAGCTGGAAC
CTAGCGGCCCCATCAGCACCATCAATCCCTGTCCTCCATGCAAAGAATGCCACAAGTGC
CCCGCTCCTAACCTGGAAGGTGGCCCAAGCGTGTTCATCTTCCCACCTAACATCAAGGA
CGTGCTGATGATCAGCCTGACACCTAAAGTGACCTGCGTGGTGGTGGACGTGTCCGAGG
ATGATCCCGATGTGCAGATCAGTTGGTTCGTGAACAACGTGGAAGTGCACACAGCCCAG
ACACAGACCCACAGAGAGGACTACAATAGCACCATTCGCGTGGTGTCCACACTGCCTAT
CCAGCACCAGGATTGGATGAGCGGCAAAGAGTTCAAGTGCAAAGTGAACAACAAGGAC
CTGCCTTCTCCAATCGAGCGGACCATCAGCAAGATCAAGGGACTCGTCAGAGCCCCTCA
GGTGTACATCTTGCCTCCACCAGCCGAGCAGCTGAGCAGAAAGGATGTGTCCCTGACCT
GTCTGGTCGTGGGCTTCAACCCTGGCGACATCAGCGTGGAATGGACCAGCAATGGCCAC
ACCGAGGAAAACTACAAGGACACAGCCCCTGTGCTGGACAGCGACGGCAGCTACTTCA
TCTACAGCAAGCTGAACATGAAGACCAGCAAGTGGGAGAAAACCGACAGCTTCTCCTG
CAACGTGCGGCACGAGGGCCTGAAGAACTACTACCTGAAGAAAACCATCTCTCGGAGC
CCCGGCAAG (SEQ ID NO: 70)
Light chain of MWTx-003:
Protein sequence (Constant region indicated by italics):
DIVMTQSHKFMSTSVGDRVSITCKASQDVTTAVAWYQQKPGQSPKILIYWATTRHTGV
PDRFTGSISGTTYILTISSVQAEDLALYYCQQHYSTPYTFGGGTKLEIKRADAAPTVSIFPP
SSEQLTSGGASVVCFLNNFYPKDINVKWKIDGSERQNGVLNSWTDQDSKDSTYSMSSTLTLTK
DEYERHNSYTCEATHKTSTSPIVKSFNRNEC (SEQ ID NO: 71)
Nucleotide sequence:
GACATCGTGATGACCCAGAGCCACAAGTTCATGAGCACCAGCGTGGGCGACAGAGTGT
CCATCACCTGTAAAGCCAGCCAGGACGTGACAACAGCCGTGGCCTGGTATCAGCAGAA
GCCTGGCCAGTCTCCTAAGATCCTGATCTACTGGGCCACCACCAGACACACCGGCGTGC
CAGATAGATTCACCGGCAGCATCAGCGGCACCACCTACATCCTGACAATCAGCTCTGTG
CAGGCCGAGGATCTGGCCCTGTACTACTGTCAGCAGCACTACAGCACCCCTTACACCTT
TGGCGGAGGCACCAAGCTGGAAATCAAGAGAGCTGACGCCGCTCCTACCGTGTCCATCT
TTCCACCTAGCAGCGAGCAGCTGACAAGCGGCGGAGCCAGCGTCGTGTGCTTCCTGAAC
AACTTCTACCCCAAGGACATCAACGTGAAGTGGAAGATCGACGGCAGCGAGAGACAGA
ACGGCGTGCTGAATAGCTGGACCGACCAGGACAGCAAGGACTCCACCTACAGCATGTC
CAGCACACTGACCCTGACCAAGGACGAGTACGAGCGGCACAACAGCTACACATGCGAG
GCCACACACAAGACCAGCACAAGCCCCATCGTGAAGTCCTTCAACCGGAACGAGTGC
(SEQ ID NO: 72)
Humanized anti-TMPRSS6 antibody variants
hzMWTx-001Var
Heavy chain of hzMWTx-001Var:
Protein sequence (Constant region indicated by italics;
N297A mutation indicated by bold):
EVQLVQSGAEVKKPGASVKVSCKASGYTFTSYWITWVRQAPGQRLEWIGNIYPGSGST
YYNEKFKSKATITRDTSSRTAYMELSSLRSEDTAVYYCAPYDADYAMDYWGQGTLVT
VSSASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYASTYRV
VSVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSV
MHEALHNHYTQKSLSLSPG (SEQ ID NO: 73)
Nucleotide sequence:
GAAGTGCAGCTGGTGCAATCTGGCGCCGAAGTGAAGAAACCTGGCGCCTCTGTGAAGG
TGTCCTGCAAGGCTTCCGGCTACACCTTTACCAGCTACTGGATCACCTGGGTCCGACAG
GCTCCTGGCCAGAGACTGGAATGGATCGGCAACATCTACCCTGGCTCCGGCTCCACCTA
CTACAACGAGAAGTTCAAGTCCAAGGCCACAATCACCCGGGACACCTCTTCCAGAACC
GCCTACATGGAACTGTCCAGCCTGAGATCTGAGGACACCGCCGTGTACTACTGCGCCCC
TTACGACGCCGACTACGCCATGGATTATTGGGGCCAGGGCACCCTGGTCACCGTGTCCT
CTGCTTCTACCAAGGGACCCAGCGTGTTCCCTCTGGCTCCTTCCAGCAAGTCTACCTCTG
GCGGAACAGCTGCTCTGGGCTGCCTGGTCAAGGACTACTTTCCTGAGCCTGTGACCGTG
TCTTGGAACTCTGGCGCTCTGACATCCGGCGTGCACACATTTCCAGCTGTGCTGCAGTCC
TCCGGCCTGTACTCTCTGTCCTCTGTCGTGACCGTGCCTTCCTCTAGCCTGGGCACCCAG
ACCTACATCTGCAATGTGAACCACAAGCCTTCCAACACCAAGGTGGACAAGAAGGTGG
AACCCAAGTCCTGCGACAAGACCCACACCTGTCCTCCATGTCCTGCTCCAGAACTGCTC
GGCGGACCTTCCGTGTTCCTGTTTCCTCCAAAGCCTAAGGACACCCTGATGATCTCTCGG
ACCCCTGAAGTGACCTGCGTGGTGGTGGATGTGTCTCACGAGGACCCAGAAGTGAAGTT
CAATTGGTACGTGGACGGCGTGGAAGTGCACAACGCCAAGACCAAGCCTAGAGAGGAA
CAGTACGCCAGCACCTACAGAGTGGTGTCCGTGCTGACAGTGCTGCACCAGGATTGGCT
GAACGGCAAAGAGTACAAGTGCAAGGTGTCCAACAAGGCCCTGCCTGCTCCTATCGAA
AAGACCATCAGCAAGGCCAAGGGCCAGCCTAGAGAACCCCAGGTTTACACCTTGCCTC
CATCTCGGGACGAGCTGACCAAGAACCAGGTGTCCCTGACCTGTCTCGTGAAGGGCTTC
TACCCCTCCGACATCGCCGTGGAATGGGAGTCTAATGGCCAGCCAGAGAACAACTACA
AGACAACCCCTCCTGTGCTGGACTCCGACGGCTCATTCTTCCTGTACTCCAAGCTGACCG
TGGACAAGTCCAGATGGCAGCAGGGCAACGTGTTCTCCTGCTCCGTGATGCACGAGGCC
CTGCACAATCACTACACACAGAAGTCCCTGTCTCTGTCCCCTGGC (SEQ ID NO: 74)
Light chain of hzMWTx-001Var:
Protein sequence (Constant region indicated by italics):
DIQMTQSPSSLSASVGDRVTITCKASQDISNYLSWFQQKPGKAPKLLIYRANRLVEGVPS
RFSGSGSGTDFTLTISSLQPEDFATYFCLQYDEFPLTFGGGTKVEIKRTVAAPSVFIFPPSD
EQLKSGTASVVCLLNNFYPREAKVQWKVDNALQSGNSQESVTEQDSKDSTYSLSSTLTLSKAD
YEKHKVYACEVTHQGLSSPVTKSFNRGEC (SEQ ID NO: 75)
Nucleotide sequence:
GACATCCAGATGACCCAGTCTCCATCCTCTCTGTCCGCCTCTGTGGGCGACAGAGTGAC
CATCACATGCAAGGCCAGCCAGGACATCTCCAACTACCTGTCCTGGTTCCAGCAGAAGC
CTGGCAAGGCTCCCAAGCTGCTGATCTACAGAGCCAACAGACTGGTGGAAGGCGTGCC
CTCCAGATTCTCCGGATCTGGCTCTGGCACCGACTTTACCCTGACAATCTCCAGCCTGCA
GCCTGAGGACTTCGCTACCTACTTCTGCCTGCAATACGACGAGTTCCCTCTGACCTTTGG
CGGAGGCACCAAGGTGGAAATCAAGCGGACAGTGGCCGCTCCTTCCGTGTTCATCTTCC
CACCTTCCGACGAGCAGCTGAAGTCCGGCACAGCTTCTGTCGTGTGCCTGCTGAACAAC
TTCTACCCTCGGGAAGCCAAGGTGCAGTGGAAGGTGGACAATGCCCTGCAGTCCGGCA
ACTCCCAAGAGTCTGTGACCGAGCAGGACTCCAAGGACAGCACCTACAGCCTGTCCTCC
ACACTGACCCTGTCCAAGGCCGACTACGAGAAGCACAAGGTGTACGCCTGCGAAGTGA
CCCATCAGGGCCTGTCTAGCCCTGTGACCAAGTCTTTCAACCGGGGCGAGTGT (SEQ ID
NO: 76)
hzMWTx-002Var
Heavy chain of hzMWTx-002Var:
Protein sequence (Constant region indicated by italics;
N297A mutation indicated by bold):
EVQLVQSGAEVKKPGASVKVSCKASGFNIKDYYIHWVRQATGQGLEWMGRIDPEDAE
SEYAPKFQGRVTITADTSTDTAYMELSSLRSEDTAVYYCTRGDSMMVTYFDYWGQGT
LVTVSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQYAST
YRVVSVLTVLHQDWLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTVDKSRWQQGNVFS
CSVMHEALHNHYTQKSLSLSPG (SEQ ID NO: 77)
Nucleotide sequence:
GAAGTGCAGCTGGTGCAATCTGGCGCCGAAGTGAAGAAACCTGGCGCCTCTGTGAAGG
TGTCCTGCAAGGCCTCTGGCTTCAACATCAAGGACTACTACATCCACTGGGTCCGACAG
GCTACCGGACAGGGACTTGAGTGGATGGGCAGAATCGACCCTGAGGACGCCGAGTCTG
AGTACGCCCCTAAGTTTCAGGGCAGAGTGACCATCACCGCCGACACCTCTACCGACACC
GCCTACATGGAACTGTCCAGCCTGAGATCTGAGGACACCGCCGTGTACTACTGCACCAG
AGGCGACTCCATGATGGTTACCTACTTCGACTACTGGGGCCAGGGCACCCTGGTCACAG
TTTCTTCCGCTTCCACCAAGGGACCCAGCGTGTTCCCTCTGGCTCCTTCCAGCAAGTCTA
CCTCTGGCGGAACAGCTGCTCTGGGCTGCCTGGTCAAGGATTACTTCCCTGAGCCTGTG
ACCGTGTCCTGGAACTCTGGCGCTCTGACATCCGGCGTGCACACCTTTCCAGCTGTGCTG
CAATCCTCCGGCCTGTACTCTCTGTCCTCCGTCGTGACCGTGCCTTCTAGCTCTCTGGGC
ACCCAGACCTACATCTGCAATGTGAACCACAAGCCTTCCAACACCAAGGTGGACAAGA
AGGTGGAACCCAAGTCCTGCGACAAGACCCACACCTGTCCTCCATGTCCTGCTCCAGAA
CTGCTCGGCGGACCTTCCGTGTTCCTGTTTCCTCCAAAGCCTAAGGACACCCTGATGATC
TCTCGGACCCCTGAAGTGACCTGCGTGGTGGTGGATGTGTCTCACGAGGACCCAGAAGT
GAAGTTCAATTGGTACGTGGACGGCGTGGAAGTGCACAACGCCAAGACCAAGCCTAGA
GAGGAACAGTACGCCTCCACCTACAGAGTGGTGTCCGTGCTGACAGTGCTGCACCAGG
ATTGGCTGAACGGCAAAGAGTACAAGTGCAAGGTGTCCAACAAGGCACTGCCCGCTCC
TATCGAAAAGACCATCTCCAAGGCCAAGGGCCAGCCTAGAGAACCCCAGGTTTACACC
TTGCCTCCATCTCGGGACGAGCTGACCAAGAACCAGGTGTCCCTGACCTGTCTCGTGAA
GGGCTTCTACCCCTCCGACATCGCCGTGGAATGGGAGTCTAATGGCCAGCCAGAGAACA
ACTACAAGACAACCCCTCCTGTGCTGGACTCCGACGGCTCATTCTTCCTGTACTCCAAGC
TGACCGTGGACAAGTCCAGATGGCAGCAGGGCAACGTGTTCTCCTGCTCCGTGATGCAC
GAGGCCCTGCACAATCACTACACACAGAAGTCTCTGTCCCTGTCTCCTGGC (SEQ ID NO:
78)
Light chain of hzMWTx-002Var:
Protein sequence (Constant region indicated by italics):
DIQMTQSPSSLSASVGDRVTITCKASQDVSTAVAWYQQKPGKAPKLLIYWAFTRHTGV
PSRFSGSGSGTDYALTISSLQPEDFATYYCQQHYRSPWTFGGGTKVEIKRTVAAPSVFIFP
PSDEQLKSGTASVVCLLNNFYPREAKVQWKVDNALQSGNSQESVTEQDSKDSTYSLSSTLTLS
KADYEKHKVYACEVTHQGLSSPVTKSFNRGEC (SEQ ID NO: 79)
Nucleotide sequence:
GACATCCAGATGACCCAGTCTCCATCCTCTCTGTCCGCCTCTGTGGGCGACAGAGTGAC
CATCACATGCAAGGCCTCTCAGGACGTGTCCACCGCCGTTGCTTGGTATCAGCAGAAGC
CTGGCAAGGCCCCTAAGCTGCTGATCTACTGGGCCTTCACCAGACACACCGGCGTGCCC
TCTAGGTTCTCCGGCTCTGGCTCTGGCACCGATTACGCTCTGACAATCTCCAGCCTGCAG
CCTGAGGACTTCGCCACCTACTACTGCCAGCAGCACTACAGAAGCCCCTGGACATTTGG
CGGAGGCACCAAGGTGGAAATCAAGCGGACAGTGGCCGCTCCTTCCGTGTTCATCTTCC
CACCTTCCGACGAGCAGCTGAAGTCCGGCACAGCTTCTGTCGTGTGCCTGCTGAACAAC
TTCTACCCTCGGGAAGCCAAGGTGCAGTGGAAGGTGGACAATGCCCTGCAGTCCGGCA
ACTCCCAAGAGTCTGTGACCGAGCAGGACTCCAAGGACAGCACCTACAGCCTGTCCTCC
ACACTGACCCTGTCCAAGGCCGACTACGAGAAGCACAAGGTGTACGCCTGCGAAGTGA
CCCATCAGGGCCTGTCTAGCCCTGTGACCAAGTCTTTCAACCGGGGCGAGTGT (SEQ ID
NO: 80)
hzMWTx-003Var
Heavy chain of hzMWTx-003Var:
Protein sequence (Constant region indicated by italics;
LALA mutation indicated by bold):
QVQLVQSGAEVKKPGASVKVSCKASGFNIEDYYMHWVRQAPGQRLEWMGRIDPEDA
ETTYSPKFQGRVTIIPDTSANTAYMELSSLRSEDTAVYYCARSIYLDPMDYWGQGTLVT
VSSASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPEAAGGPSV
FLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDKSRWQQGNVFSCS
VMHEALHNHYTQKSLSLSPG (SEQ ID NO: 81)
Nucleotide sequence:
CAGGTGCAGCTGGTGCAGTCTGGCGCCGAAGTGAAAAAGCCTGGCGCCTCTGTGAAGG
TGTCCTGCAAGGCCTCTGGCTTCAACATCGAGGACTACTACATGCACTGGGTCCGACAG
GCCCCTGGCCAGAGATTGGAATGGATGGGCAGAATCGACCCCGAGGACGCCGAGACAA
CCTACTCTCCTAAGTTCCAGGGCCGCGTGACAATCATCCCTGACACCTCTGCCAACACC
GCCTACATGGAACTGTCCAGCCTGAGATCTGAGGACACCGCCGTGTACTACTGCGCCCG
GTCTATCTACCTGGACCCTATGGACTATTGGGGCCAGGGCACCCTGGTCACAGTGTCCT
CTGCTTCTACCAAGGGACCCAGCGTGTTCCCTCTGGCTCCTTCCAGCAAGTCTACCTCTG
GCGGAACAGCTGCTCTGGGCTGCCTGGTCAAGGACTACTTTCCAGAGCCTGTGACCGTG
TCCTGGAACTCTGGCGCTCTGACATCTGGCGTGCACACCTTTCCAGCTGTGCTGCAGTCC
TCCGGCCTGTACTCTCTGTCCTCTGTCGTGACCGTGCCTTCCAGCTCTCTGGGAACCCAG
ACCTACATCTGCAATGTGAACCACAAGCCTTCCAACACCAAGGTGGACAAGAAGGTGG
AACCCAAGTCCTGCGACAAGACCCACACCTGTCCTCCATGTCCTGCTCCAGAAGCTGCT
GGCGGCCCTTCCGTGTTTCTGTTCCCTCCAAAGCCTAAGGACACCCTGATGATCTCTCGG
ACCCCTGAAGTGACCTGCGTGGTGGTGGATGTGTCTCACGAGGACCCAGAAGTGAAGTT
CAATTGGTACGTGGACGGCGTGGAAGTGCACAACGCCAAGACCAAGCCTAGAGAGGAA
CAGTACAACTCCACCTACAGAGTGGTGTCCGTGCTGACCGTGCTGCACCAGGATTGGCT
GAACGGCAAAGAGTACAAGTGCAAGGTGTCCAACAAGGCACTGCCCGCTCCTATCGAA
AAGACCATCTCCAAGGCCAAGGGCCAGCCTAGGGAACCCCAGGTTTACACCCTGCCTCC
AAGCCGGGAAGAGATGACCAAGAACCAGGTGTCCCTGACCTGCCTCGTGAAGGGCTTC
TACCCTTCCGACATCGCCGTGGAATGGGAGAGCAATGGCCAGCCAGAGAACAACTACA
AGACAACCCCTCCTGTGCTGGACTCCGACGGCTCATTCTTCCTGTACTCCAAGCTGACA
GTGGACAAGTCCAGATGGCAGCAGGGCAACGTGTTCTCCTGCTCCGTGATGCACGAGGC
CCTGCACAATCACTACACACAGAAGTCCCTGTCTCTGTCCCCTGGC (SEQ ID NO: 82)
Light chain of hzMWTx-003Var:
Protein sequence (Constant region indicated by italics):
DIQMTQSPKSLSASVGDRVTITCRASQDVTTALAWYQQKPGQSPKLLIYWATTRHSGV
PSRFSGSGSGTDFTLTISSLQPEDFATYYCQQHYSTPYTFGQGTKLEIKRTVAAPSVFIFPP
SDEQLKSGTASVVCLLNNFYPREAKVQWKVDNALQSGNSQESVTEQDSKDSTYSLSSTLTLSK
ADYEKHKVYACEVTHQGLSSPVTKSFNRGEC (SEQ ID NO: 83)
Nucleotide sequence:
GACATCCAGATGACCCAGTCTCCAAAGTCTCTGTCCGCCTCCGTGGGCGACAGAGTGAC
CATCACCTGTAGAGCCTCTCAGGACGTGACCACCGCTCTGGCTTGGTATCAGCAGAAGC
CTGGCCAGTCTCCTAAGCTGCTGATCTACTGGGCCACCACCAGACACTCTGGCGTGCCC
TCTAGATTCTCCGGCTCTGGCTCTGGCACCGACTTTACCCTGACAATCTCCAGCCTGCAG
CCTGAGGACTTCGCCACCTACTACTGCCAGCAGCACTACAGCACCCCTTACACCTTTGG
CCAGGGCACCAAGCTGGAAATCAAGCGGACAGTGGCCGCTCCTTCCGTGTTCATCTTCC
CACCTTCCGACGAGCAGCTGAAGTCCGGCACAGCTTCTGTCGTGTGCCTGCTGAACAAC
TTCTACCCTCGGGAAGCCAAGGTGCAGTGGAAGGTGGACAATGCCCTGCAGTCCGGCA
ACTCCCAAGAGTCTGTGACCGAGCAGGACTCCAAGGACAGCACCTACAGCCTGTCCTCC
ACACTGACCCTGTCCAAGGCCGACTACGAGAAGCACAAGGTGTACGCCTGCGAAGTGA
CCCATCAGGGCCTGTCTAGCCCTGTGACCAAGTCTTTCAACCGGGGCGAGTGT (SEQ ID
NO: 84)

### Dose-Dependent Effects of Anti-TMPRSS6 Antibodies on HAMP Promoter Activity

FIGS. 2A-2F show the results from using the HAMP-luciferase report assay described above to test MWTx-001, MWTx-002, MWTx-003 and their humanized variants hzMWTx-001Var, hzMWTx-002Var, hzMWTx-003Var, respectively, at the indicated concentrations. Each of MWTx-001 (FIG. 2A), MWTx-002 (FIG. 2B3), MWTx-003 (FIG. 2C) and humanized variants hzMWTx-001Var (FIG. 2D3), hzMWTx-002Var (FIG. 2E), hzMWTx-003Var (FIG. 2F⁷) increases HAMP promoter activity in a dose-dependent manner. The EC₅₀ for MWTx-001 was calculated to be 3 μg/ml (FIG. 2A). The EC₅₀ for MWTx-002 was calculated to be 1 μg/ml (FIG. 2B3). The EC₅₀ for MWTx-003 was calculated to be 2 μg/ml (FIG. 2C). The EC₅₀ for hzMWTx-001Var was calculated to be 0.8 μg/ml (FIG. 2D). The EC₅₀ for hzMWTx-002Var was calculated to be 0.3 μg/ml (FIG. 2E). The EC₅₀ for hzMWTx-003Var was calculated to be 0.3 μg/ml (FIG. 2F).

### Example 3. Binding Affinity of Anti-TMPRSS6 Antibodies

The binding affinity of various anti-TMPRSS6 antibodies to human TMPRSS6 expressed on HEK293T cells was measured using three different methods: cell surface ELISA (FIGS. 3A-3C), FACS (FIGS. 3D-3F), and Bio-Layer Interferometry (FIGS. 3G-3M).

### Anti-TMPRSS6 mAb Binding Affinity Measurement Using Cell Surface ELISA

HEK293T cells stably expressing human TMPRSS6 (generated by LakePharma Inc as described above; SEQ ID NO: 97) were fixed with 4% paraformaldehyde (PFA), and washed with dPBS (Dulbecco's phosphate-buffered saline, Corning Cellgro) before incubation with various concentrations of anti-TMPRSS6 antibodies diluted in BSA medium (DMEM+1% Pen/Strep+10 mM HEPES+1 mg/ml BSA (Sigma-Aldrich). Purified mouse IgG was used as a control (Sigma-Aldrich). After incubation, cells were washed with BSA medium and then incubated with goat anti-mouse IgG conjugated with HRP as a 2° antibody (Invitrogen). At last, cells were washed with dPBS to remove unbound antibody and color developed with ELISA liquid substrate (Sigma-Aldrich), followed by stopping the reaction with addition of the same volume of ELISA liquid substrate of 1M H₂SO₄. Bound antibody was measured by absorbance at OD_{450 nm}. Results for these assays are shown in FIGS. 3A-3C.

### Anti-TMPRSS6 mAb Binding Affinity Measurement Using FACS

HEK293T cells stably expressing human TMPRSS6 were collected, and blocked with dPBS+3% BSA before incubation with various concentrations of anti-TMPRSS6 antibodies diluted in dPBS+3% BSA. Purified mouse IgG was used as a control. After incubation, cells were washed with dPBS and then incubated with goat anti-mouse IgG conjugated with APC as a 2° antibody (Jackson ImmunoResearch Inc). At last, cells were washed with dPBS to remove unbound antibody, re-suspended with dPBS+1 mM EDTA, and then subjected to FACS analysis using a NOVOCYTE® Flow Cytometer (ACEA Biosciences, Inc., San Diego CA). Bound antibody was determined by measuring mean APC intensity after excitation at 640 nm and measurement of emission (fluorescence) at 675 nm. Results for these assays are shown in FIGS. 3D-3F.

### Anti-TMPRSS6 Antibody Affinity and Binding Kinetics Measurement Using Bio-Layer Interferometry

Bio-Layer Interferometry technology was used for anti-TMPRSS6 antibody affinity measurement and binding kinetics determinations with Octet® RED96e system (Sartorius AG). Pre-hydrated Anti-Mouse IgG Fc Capture (AMC) biosensors (for MWTx-001, MWTx-002 and MWTx-003 anti-TMPRSS6 antibodies, FIGS. 3G-3I) or Anti-Human IgG Fc Capture (AHC) biosensors (for hzMWTx-001Var, hzMWTx-002Var and hzMWTx-003Var anti-TMPRSS6 antibodies, FIGS. 3J-3L) were first equilibrated in 1×KB (Kinetic Buffer, 1×PBS pH 7.4+0.02% Tween-20+0.1% BSA) for 120 sec for the first baseline, followed by loading with 10 mg/ml anti-TMPRSS6 antibody (MWTx-001, FIG. 3G; MWTx-002, FIG. 3H; MWTx-003, FIG. 3I; hzMWTx-001Var, FIG. 3J; hzMWTx-002Var, FIG. 3K; hzMWTx-003Var, FIG. 3L) onto AMC or AHC biosensors for 240 sec. Then, the second baseline signal was established for 120 sec before association with various concentrations of human ecto-TMPRSS6-FLAG (SEQ ID NO: 102) (generated in house by fusing extracellular domain of human TMPRSS6 with a FLAG-tag at C-terminus) for 240 sec. At last, analyte was dissociated in 1×KB for 360 sec. Data analysis was done using Octet Data Analysis HT Software. KD, kₒₙ, k_{off} and R² were summarized in FIG. 3M.

### Example 4: Cross-Reactivity: Anti-TMPRSS6 Antibody Binding to Human TMPRSS6 and Non-Human TMPRSS6

### Cross-Reactivity Determination by FACS

Selected anti-TMPRSS6 antibodies were tested to determine whether any are capable of binding to TMPRSS6 from mouse and/or cynomolgus monkey. HEK293T cells stably expressing human TMPRSS6 (HuTMPRSS6-(His)₆) (generated by LakePharma Inc as described above), HEK293T cells stably expressing mouse TMPRSS6 (MoTMPRSS6-(His)₆) (SEQ ID NO: 98) (generated by LakePharma Inc as described above) and HEK293T cells transiently expressing cynomolgus monkey TMPRSS6 (CynoTMPRSS6-(His)₆) (SEQ ID NO: 99) (generated in house) were collected. HEK293T cells stably expressing human TMPRSS6 were used as a positive control and HEK293T cells were used as a negative control (as described above). Cells were blocked with dPBS+3% BSA before incubation with anti-TMPRSS6 antibodies diluted in dPBS+3% BSA. After incubation, cells were washed with dPBS and followed by another incubation with goat anti-mouse IgG conjugated with AlexaFluor-488 as a 2° antibody (Invitrogen). At last, cells were washed with dPBS to remove unbound antibody, re-suspended with dPBS+1 mM EDTA, and then subjected to FACS analysis using a NOVOCYTE® Flow Cytometer (ACEA Biosciences, Inc., San Diego CA). Bound antibody was determined by excitation at 488 nm and measurement of emission (FITC-A) at 530 nm. Results for these assays are shown in histogram plots in FIGS. 4A-4I. Cross-reactivity with mouse TMPRSS6 was observed for MWTx-001 (FIG. 4D) and MWTx-003 (FIG. 4F), whereas MWTx-002 (FIG. 4E) did not show detectable cross-reactivity with mouse TMPRSS6. Cross-reactivity with cynomolgus monkey TMPRSS6 was observed for MWTx-001 (FIG. 4G), MWTx-002 (FIG. 4H) and MWTx-003 (FIG. 4I).

### Cross-Reactivity Determination by Cell Surface ELISA

HEK293T cells stably expressing mouse TMPRSS6 (generated by LakePharma Inc as described above, FIGS. 4J, 4L, 4N, 4P, 4R, 4T) or cynomolgus monkey (generated in house as described above, FIGS. 4K, 4M, 4O, 4Q, 4S, 4U) were fixed with methanol (100%), and washed with dPBS (Dulbecco's phosphate-buffered saline, Corning Cellgro) before incubation with various concentrations of anti-TMPRSS6 antibodies and their humanized variants diluted in BSA medium (DMEM+1% Pen/Strep+10 mM HEPES+1 mg/ml BSA (Sigma-Aldrich)). Purified mouse IgG (FIGS. 4J-4O) or Human IgG1 (FIGS. 4P-4U) was used as a control. After incubation, cells were washed with BSA medium and then incubated with goat anti-mouse (Invitrogen, FIGS. 4J-4O) or anti-human (Millipore, FIGS. 4P-4U) IgG conjugated with HRP as a 2° antibody. Finally, cells were washed with dPBS to remove unbound antibody and color developed with ELISA liquid substrate (Sigma-Aldrich), followed by stopping the reaction with addition of the same volume of ELISA liquid substrate of 1M H₂SO₄. Bound antibody was measured by absorbance at OD_{450 nm}. Results for these assays are shown in FIGS. 4J-4U. Cross-reactivity with mouse TMPRSS6 was observed for MWTx-001 (FIG. 4J) and MWTx-003 (FIG. 4N) anti-TMPRSS6 antibodies and their humanized variants hzMWTx-001Var (FIG. 4P), and hzMWTx-003Var (FIG. 4T) anti-TMPRSS6 antibodies, whereas MWTx-002 (FIG. 4L) anti-TMPRSS6 antibody or its humanized variant hzMWTx-002Var (FIG. 4R) anti-TMPRSS6 antibody did not show detectable cross-reactivity with mouse TMPRSS6. Cross-reactivity with cynomolgus monkey TMPRSS6 was observed for MWTx-001 (FIG. 4K), MWTx-002 (FIG. 4M), and MWTx-003 (FIG. 4O) anti-TMPRSS6 antibodies and their humanized variants hzMWTx-001Var (FIG. 4Q), hzMWTx-002Var (FIG. 4S), and hzMWTx-003Var (FIG. 4U) anti-TMPRSS6 antibodies.

### Example 5: Target Specificity: Anti-TMPRSS6 Antibody Binding to Homologous Matriptases

To determine if anti-TMPRSS6 antibodies bind to homologous matriptases, HEK293T cells over-expressing matriptase (ST14) (SEQ ID NO: 100) (FIGS. 5B, 5E, 5H, 5K, 5N, 5Q), and HEK293T cells over-expressing matriptase-3 (TMPRSS7) (SEQ ID NO: 101) (FIGS. 5C, 5F, 5I, 5L, 50, 5R) were collected (generated in house). HEK293T cells stably expressing human TMPRSS6 (matriptase-2) (SEQ ID NO:97) (generated by LakePharma Inc as described above, FIGS. 5A, 5D, 5G, 5J, 5M, 5P) were used as a positive control and HEK293T cells (FIGS. 5A-5R) were used as a negative control (as described above). Cells were blocked and permeabilized with dPBS+3% BSA+0.1% Tween-20 before incubation with various anti-TMPRSS6 antibodies diluted in dPBS+3% BSA+0.1% Tween-20. Cells were incubated with anti-TMPRSS6 antibodies and their humanized variants at a concentration of roughly 1 μg/ml for 1 hr. After incubation, cells were washed with dPBS and incubated with goat anti-mouse IgG conjugated with AlexaFluor-488 (Invitrogen, FIGS. 5A-5I) or goat anti-human IgG conjugated with Allophycocyanin (APC) (Jackson Immuno Research, FIGS. 5J-5R) as a 2° antibody. At last, cells were washed with dPBS and re-suspended with dPBS+1 mM EDTA, and then subjected to FACS analysis using a NOVOCYTE® Flow Cytometer. Bound antibody was determined by excitation at 488 nm and measurement of emission (FITC-A) at 530 nm (FIGS. 5A-5I) or by excitation at 640 nm and measurement of emission (APC-A) at 675 nm (FIGS. 5J-5R). Results for these assays are shown in histogram plots in FIGS. 5A-5R. All of the antibodies showed binding to human TMPRSS6 (matriptase-2) (FIGS. 5A, 5D, 5G, 5J, 5M, 5P) and none of the antibodies showed binding to homologous matriptases ST14 (FIGS. 5B, 5E, 5H, 5K, 5N, 5Q) or TMPRSS7 (FIGS. 5C, 5F, 5I, 5L, 50, 5R). MWTx-001 anti-TMPRSS6 antibody and its humanized variant hzMWTx-001Var anti-TMPRSS6 antibody showed binding to human TMPRSS6 (FIGS. 5A, 5J) and did not show binding to matriptase (ST14) (FIGS. 5B, 5K) or matriptase-3 (TMPRSS7) (FIGS. 5C, 5L). MWTx-002 anti-TMPRSS6 antibody and its humanized variant hzMWTx-002Var anti-TMPRSS6 antibody showed binding to human TMPRSS6 (matriptase-2) (FIGS. 5D, 5M) and did not show binding to matriptase (ST14) (FIGS. 5E, 5N) or matriptase-3 (TMPRSS7) (FIGS. 5F, 5O). MWTx-003 anti-TMPRSS6 antibody and its humanized variant hzMWTx-003Var anti-TMPRSS6 antibody showed binding to human TMPRSS6 (matriptase-2) (FIGS. 5G, 5P) and did not show binding to matriptase (ST14) (FIGS. 5H, 5Q) or matriptase-3 (TMPRSS7) (FIGS. 5I, 5R).

### Example 6. Treatment with Anti-TMPRSS6 Antibodies in a Mouse Pharmacodynamic Model

In order to study the in vivo pharmacodynamic responses of anti-TMPRSS6 antibodies, 2-10 mg/kg of MWTx-003 anti-TMPRSS6 antibody (FIGS. 6A-6B, 6D-6E, 6G-6H, 6J-6K) or its humanized variant hzMWTx-003Var anti-TMPRSS6 antibody (FIGS. 6C, 6F, 6I, 6L) was injected intraperitoneally into wildtype C57BL/6J mouse. Mouse IgG2b (BioXcell, FIGS. 6A-6B, 6D-6E, 6G-6H, 6J-6K) or human IgG1 (BioXcell, FIGS. 6C, 6F, 6I, 6L) was used as isotype control. 20 hours post injection, 50 μg of GFP-TMPRSS6 plasmid DNA (generated in house by inserting human TMPRSS6 into a GFP vector) was delivered into each mouse by hydrodynamic tail vein injection. 44 hours post hydrodynamic injection, mice were euthanized, liver tissues and blood were collected. Liver RNA was purified by EZgene Total RNA Purification Plus from Biomiga (San Diego, CA) according to the manufacturer's instructions. Mouse serum was obtained by centrifugation of whole blood at 1500×g, 10 min.

### Effects of Treatment with Anti-TMPRSS6 Antibodies on Serum Iron

Serum iron was measured by a chromogenic assay developed in house (FIGS. 6A-6C). Briefly, mouse serum or iron standard (31-500 μg/dL) was mixed with Mixed Acid Solution (0.6M Trichloroacetic acid, 0.4M Thioglycolic sodium, 1M HCl) by vertexing for 30 sec. The mixtures were incubated for 10 min at 37° C. before centrifugation at 10,000×g for 10 min followed by color development in Color Solution (1.5M Sodium Acetate, 0.5 mM Bathophenanthroline disulfonic salt). The absorbance was then read at OD_{535 nm}. The serum iron concentration was calculated from linear iron standard curve. Treatment of 10 mg/kg MWTx-003 anti-TMPRSS6 antibody (FIGS. 6A-6B) and its humanized variant hzMWTx-003Var anti-TMPRSS6 antibody (FIG. 6C) significantly reduced serum iron.

### Effects of Treatment with Anti-TMPRSS6 Antibodies on Serum Hepcidin

Serum hepcidin was measured by Hepcidin-Murine Compete ELISA kit purchased from Intrinsic Lifesciences (La Jolla, CA) according to the manufacturer's instructions (FIGS. 6D-6F). Briefly, diluted mouse serum or hepcidin standard was mixed with hepcidin biotin conjugate before adding to the plate coated with an anti-murine hepcidin antibody. Serum hepcidin or hepcidin standard competes with hepcidin biotin conjugate for binding to coated anti-hepcidin antibody. The bound hepcidin biotin conjugate was detected with streptavidin conjugated horseradish peroxidase (HRP), and color developed with TMB followed by stop solution. The absorbance was then read at OD_{450 nm}. The data was analyzed with Graphpad Prism 8 using a four-parameter logistic (4-PL) curve-fit, and serum hepcidin concentration was interpolated. Hydrodynamic delivery of GFP-TMPRSS6 significantly reduced serum hepcidin level (FIG. 6D), whereas treatment with 10 mg/kg MWTx-003 anti-TMPRSS6 antibody (FIGS. 6D-6E) and its humanized variant hzMWTx-003Var anti-TMPRSS6 antibody (FIG. 6F) reversed the repression of hepcidin and significantly increased serum hepcidin level.

### Effects of Treatment with Anti-TMPRSS6 Antibodies on Liver Hepcidin RNA

Liver hepcidin RNA was quantified by real-time qPCR (FIGS. 6G-6I). Briefly, cDNA was first synthesized from liver RNA using iScript Reverse Transcription Supermix (Bio-Rad) according to the manufacturer's instructions. Hepcidin transcripts were amplified with specific primers listed below in Table 4, and detected using SsoAdvanced™ Universal SYBR® Green Supermix (Bio-Rad) according to the manufacturer's instructions on Bio-Rad CFX96 qPCR instrument. Samples were analyzed in triplicate, and results are normalized to β-actin RNA levels (measured by transcription, amplification with primers listed in Table 4, and quantification as described above). Hydrodynamic delivery of GFP-TMPRSS6 significantly reduced liver hepcidin RNA (FIG. 6G). Treatment of 10 mg/kg MWTx-003 anti-TMPRSS6 antibody (FIGS. 6G-6H) and its humanized variant hzMWTx-003Var anti-TMPRSS6 antibody (FIG. 61) reversed the repression of Hamp and significantly increased liver hepcidin RNA levels. The following primers were used for RNA quantification by real-time qPCR: Hepcidin forward primer: 5′-AAG CAG GGC AGA CAT TGC GAT-3′ (SEQ ID NO: 85); Hepcidin reverse primer: 5′-CAG GAT GTG GCT CTA GGC TAT-3′ (SEQ ID NO: 86); β-actin forward primer: 5′-ACC CAC ACT GTG CCC ATC TA-3′ (SEQ ID NO: 87); β-actin reverse primer: 5′-CAC GCT CGG TCA GGA TCT TC-3′ (SEQ ID NO: 88).

Serum concentration of MWTx-003 anti-TMPRSS6 antibody or its humanized variant hzMWTx-003Var anti-TMPRSS6 antibody was quantified by cell surface ELISA developed in house (as described above, FIG. 6J-6L). Briefly, diluted mouse serum or anti-TMPRSS6 antibody standard were incubated with 100% methanol fixed HEK293T cells stably expressing human TMPRSS6 (HEK293T cells were used as background control). Bound MWTx-003 anti-TMPRSS6 antibody was detected with goat anti-mouse IgG conjugated with HRP, and bound hzMWTx-003Var anti-TMPRSS6 antibody was detected with goat anti-human IgG conjugated with HRP. Color was developed with TMB followed by stop solution. The absorbance was then read at OD_{450 nm}. Samples were analyzed in triplicate, and results are normalized to HEK293T control. The data was analyzed with Graphpad Prism 8 using a four-parameter logistic (4-PL) curve-fit, and serum anti-TMPRSS6 antibody concentration was interpolated.

### Example 7. In Vivo Efficacy of Anti-TMPRSS6 Antibody Using Beta-Thalassemia Mouse Model

In order to study in vivo efficacy of anti-TMPRSS6 antibody, a β-thalassemia mouse model (B6.129P2-Hbb-b1^{tm1Unc} Hbb-b2^{tm1Unc}/J, JAX Stock No: 002683, The Jackson Laboratories, Bar Harbor ME) was chosen, herein referred to as Th3/+ mouse. 4-5 weeks old Th3/+ mice and their wildtype (WT) littermates were put on an iron sufficient diet (Teklad TD.80394) and Th3/+ mice were treated with 10 mg/kg MWTx-003 anti-TMPRSS6 antibody or mouse IgG2b isotype control every three days for 4 weeks, while WT littermates did not receive treatments. At the end of the treatment course, mice were euthanized, and spleen, liver, femur and blood samples were collected. Liver total RNA was purified, and serum was collected as described above.

### Effects on Blood Counts, Splenomegaly, Serum Iron, Serum Hepcidin, Liver Hepcidin RNA

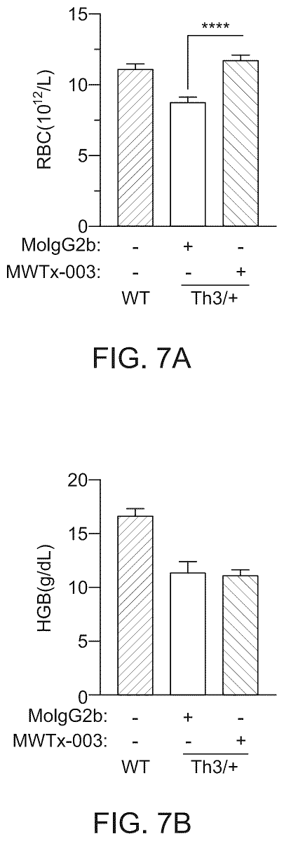

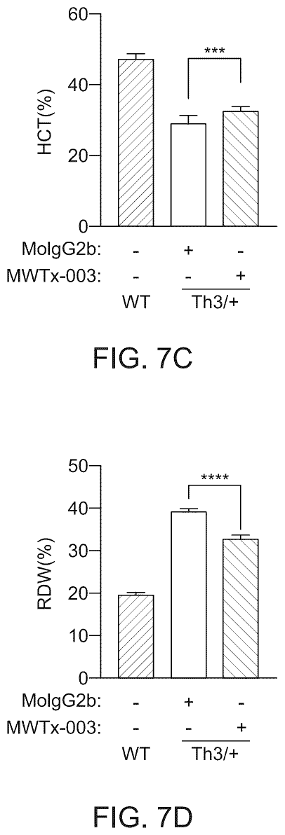

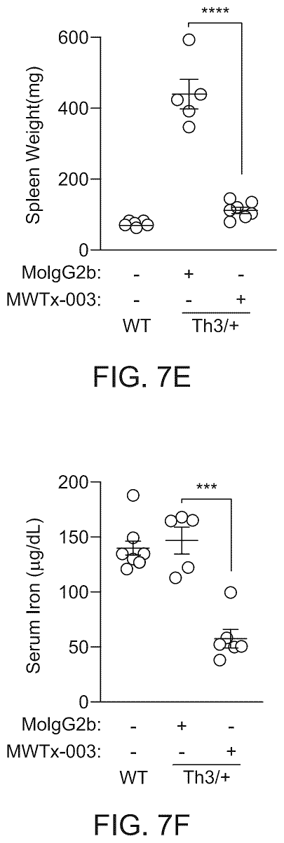

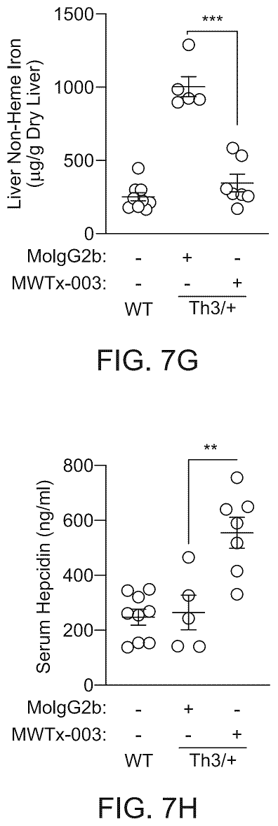

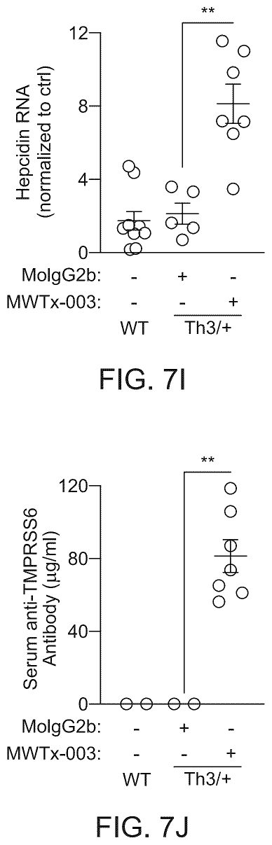

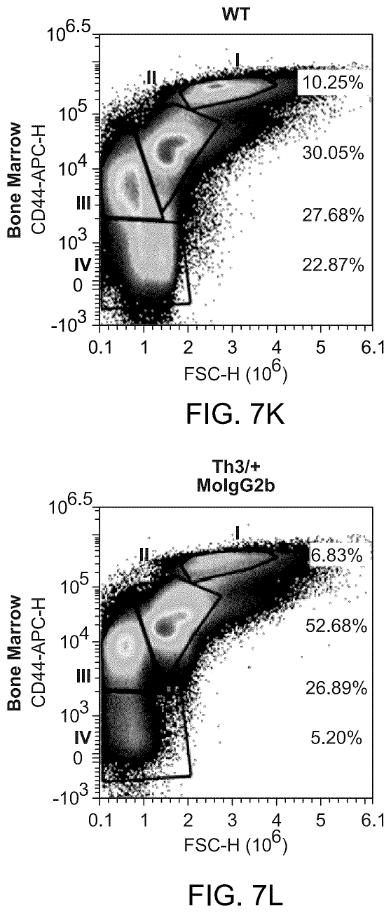

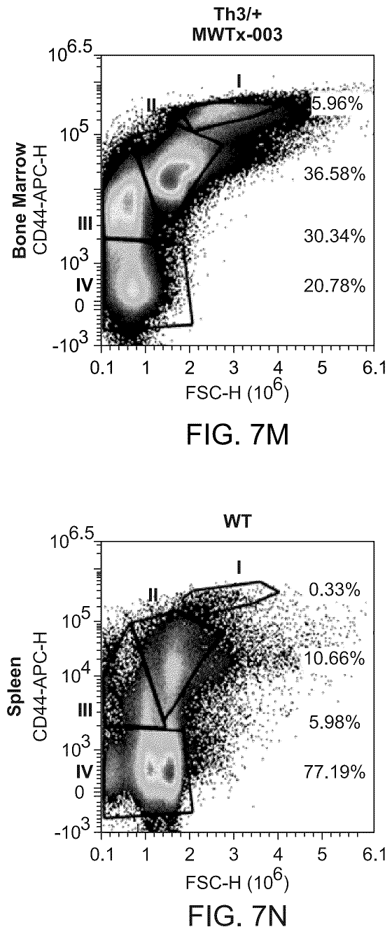

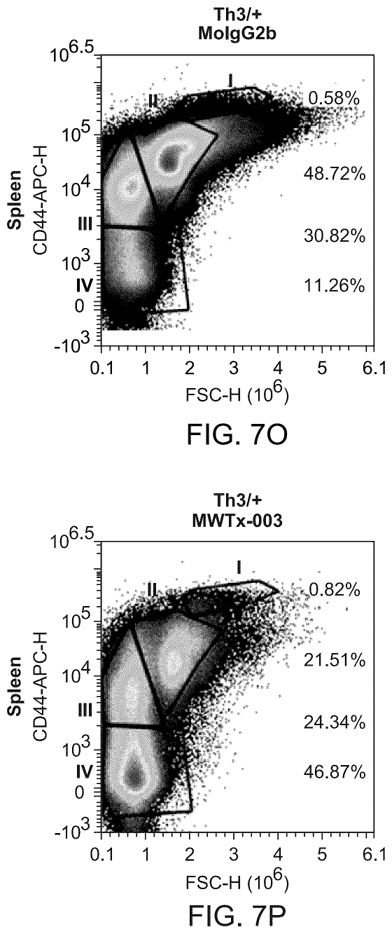

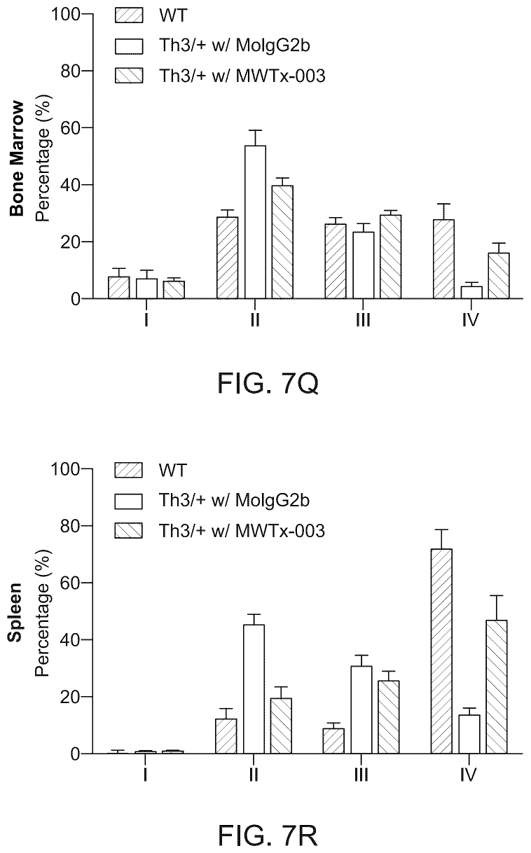

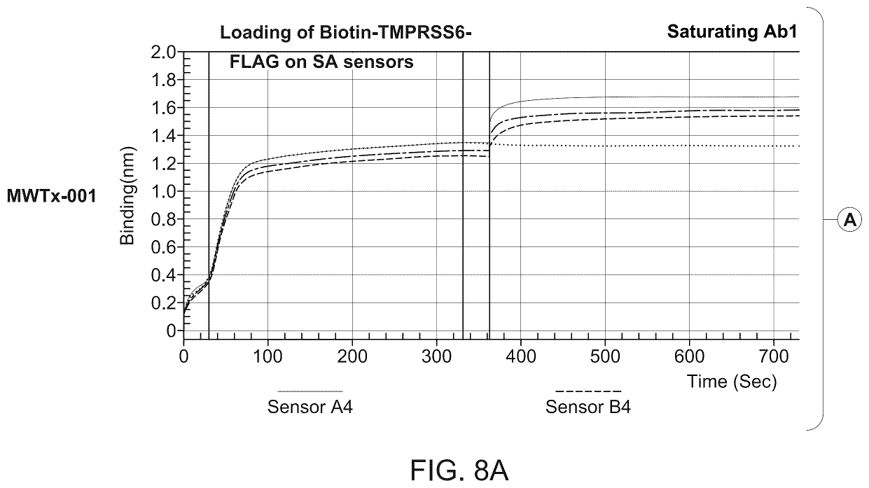

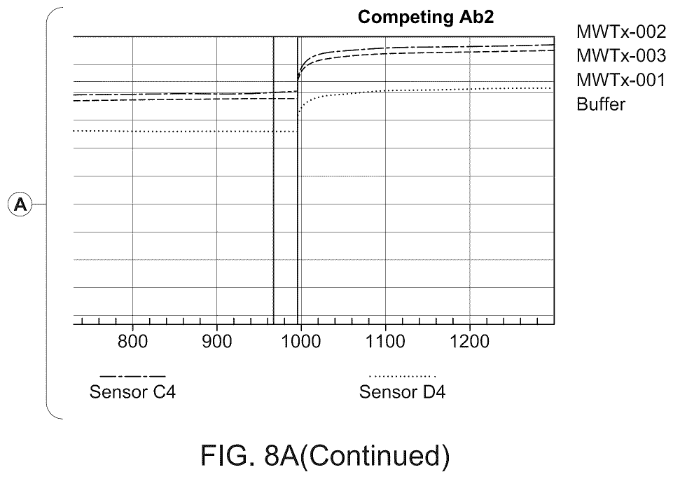

Complete Blood Count (CBC) was performed by VETSCAN HM5 automated hematology analyzer (FIGS. 7A-7D). MWTx-003 anti-TMPRSS6 antibody treatment significantly increased Red Blood Count (RBC, FIG. 7A) and hematocrit (HCT, FIG. 7C), and reduced Red Cell Distribution Width (RDW, FIG. 7D), but had no apparent effect on Hemoglobin (HGB, FIG. 7B) in Th3/+ mice.

Spleen weight was measured, and MWTx-003 anti-TMPRSS6 antibody treatment significantly reduced splenomegaly for Th3/+ mice (FIG. 7E).

Serum iron was measured as described above. Treatment with MWTx-003 anti-TMPRSS6 antibody significantly reduced serum iron (FIG. 7F). Liver non-heme iron was measured using a similar chromogenic assay (FIG. 7G). Briefly, minced small liver tissue was dried at 65° C. for overnight followed by digestion with mixed acid (3M HCl, 10% Trichloroacetic acid) at 65° C. for 20 hr. Then, digestion supernatant was collected for color development in Color Solution (1.5M sodium acetate, 0.5 mM bathophenanthroline disulfonic salt). The absorbance was then read at OD_{535 nm}. Treatment with MWTx-003 anti-TMPRSS6 antibody significantly reduced liver non-heme iron (FIG. 7G).

Serum hepcidin was measured by Hepcidin-Murine Compete ELISA kit as described above. Treatment with MWTx-003 anti-TMPRSS6 antibody significantly increased serum hepcidin (FIG. 7H).

Liver hepcidin RNA was quantified by real-time qPCR as described above. Treatment with MWTx-003 anti-TMPRSS6 antibody significantly increased liver hepcidin RNA (FIG. 7I).

Serum concentration of MWTx-003 anti-TMPRSS6 antibody was quantified by cell surface ELISA developed in-house as described above (FIG. 7J).

### Effects on Erythropoiesis

In order to study effects of MWTx-003 anti-TMPRSS6 antibody on erythropoiesis in Th3/+ mice, bone marrow was harvested from femur (see FIGS. 7K-7M) and splenocytes were harvested from spleen (see FIGS. 7N-7P), and analyzed. Harvested cells were blocked with rat anti-mouse CD16/CD32 (BD Biosciences) for 15 min followed by staining with rat anti-mouse TER119 conjugated with FITC (BD Biosciences) and rat anti-mouse CD44 conjugated with APC (Invitrogen) for 30 min on ice. Washed cells were stained with the viability marker 7-AAD (BD Biosciences) for 10 min on ice before FACS analysis using a NOVOCYTE® Flow Cytometer. Ter 19⁺, 7-ADD⁻ cells were selected, and density plots were graphed with anti-mouse CD44 over cell size (FSC-H). Plots were analyzed to identify cell types (cell clusters) and determine the abundance of each type (cluster) Representative plots in FIGS. 7K-7P show that four distinct cell clusters were distinguished from top to bottom, corresponding to successive stages in erythroid differentiation and identified as: basophilic erythroblasts (cluster I), polychromatic erythroblasts (cluster II), orthochromatic erythroblasts and nonnucleated reticulocytes (cluster III) and mature red cells (cluster IV). Percentage (%) value of each cluster in a sample was calculated as a measure of the abundance of cell type(s) in that cluster, as shown in FIGS. 7K-7P. The % value for each cell cluster (I), (II), (III), (IV) was calculated for each sample (bone marrow, spleen) from each animal in each treatment course, as follows: WT (no treatment) N=9; disease model Th3/+ mouse treated with IgG2b isotype control (Th3+ w/MoIgG2b), N=5; disease model Th3/+ mouse treated with MWTx-003 anti-TMPRSS6 antibody (Th3+ w/MWTx-003), N=7 and average values were then calculated. On average, populations of basophilic erythroblasts (I) showed a 7.58% (Th3+ w/MoIgG2b) to 6.52% (Th3+ w/MWTx-003) shift (7.96% for WT), polychromatic erythroblasts (II) showed a 54.20% (Th3+ w/MoIgG2b) to 40.01% (Th3+ w/MWTx-003) shift (28.53% for WT), orthochromatic erythroblasts and nonnucleated reticulocytes (III) showed a 24.06% (Th3+ w/MoIgG2b) to 29.73% (Th3+ w/MWTx-003) shift (26.67% for WT) and mature red cells (IV) showed a 4.54% (Th3+ w/MoIgG2b) to 16.44% shift (27.66% for WT) in bone marrow cells after four weeks. On average, populations of basophilic erythroblasts (I) showed a 0.71% (Th3+ w/MoIgG2b) to 0.91% (Th3+ w/MWTx-003) shift (0.46% for WT), polychromatic erythroblasts (II) showed a 45.76% (Th3+ w/MoIgG2b) to 19.25% (Th3+ w/MWTx-003) shift (12.23% for WT), orthochromatic erythroblasts and nonnucleated reticulocytes (III) showed a 31.16% (Th3+ w/MoIgG2b) to 28.72% (Th3+ w/MWTx-003) shift (8.67% for WT) and a mature red cells (IV) showed a 14.13% (Th3+ w/MoIgG2b) to 44.38% (Th3+ w/MWTx-003) shift (72.17% for WT) in spleen after found weeks. These results are shown in a bar graph in FIG. 7Q for bone marrow, and FIG. 7R for spleen.

In Th3/+ mice, treatment with MWTx-003 anti-TMPRSS6 antibody improved ineffective erythropoiesis, with a significant proportion of erythroblasts differentiated and matured into red blood cells.

### Example 8. Anti-TMPRSS6 Antibodies Epitope Binning

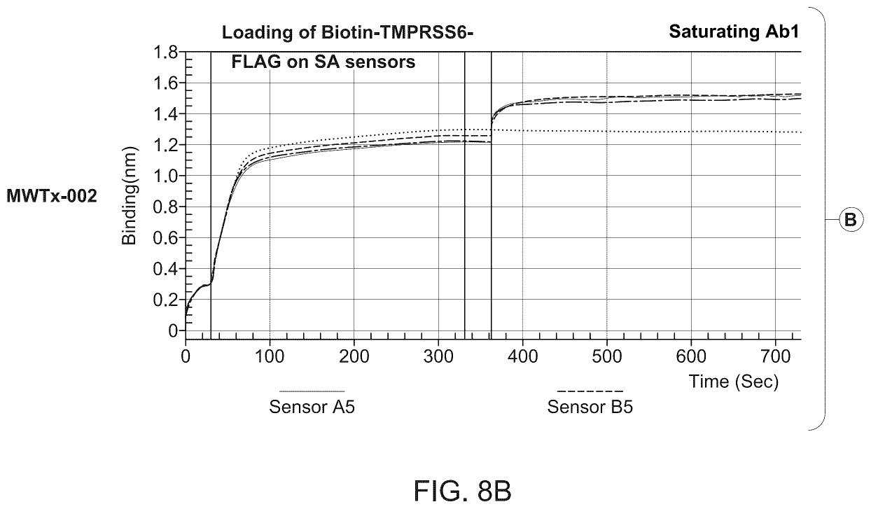

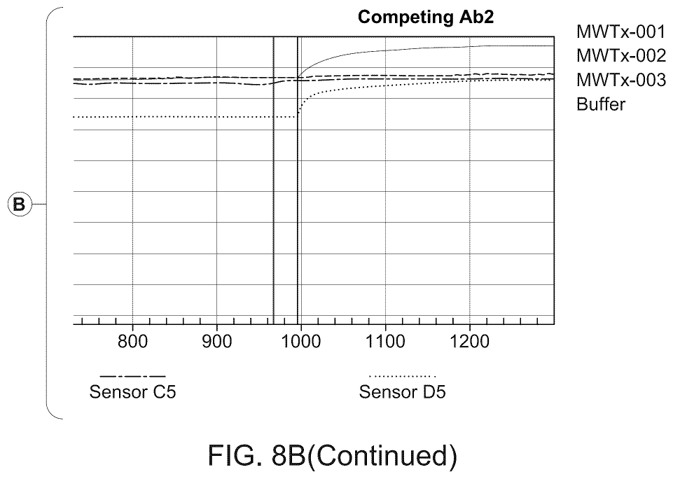

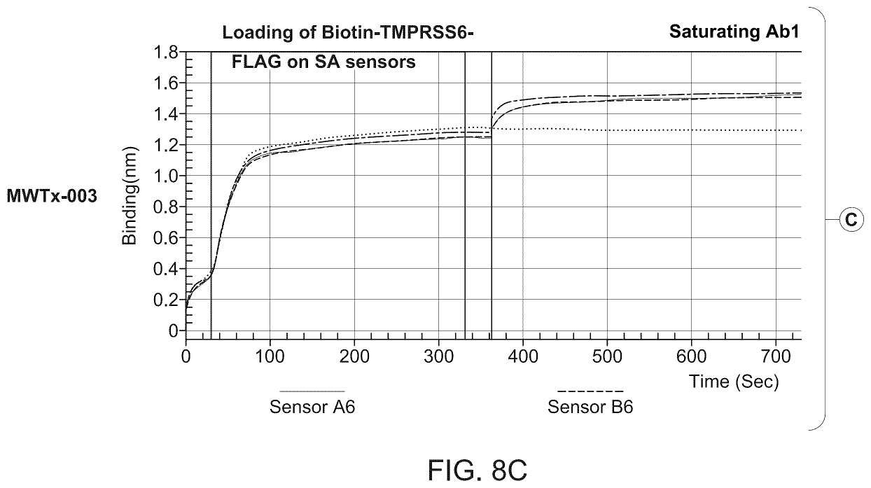

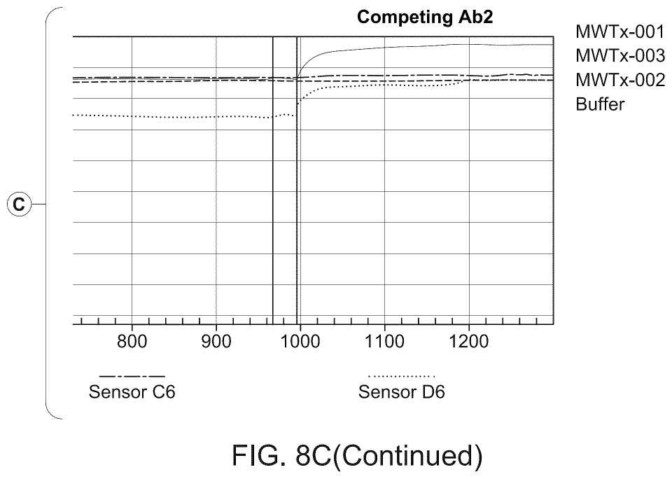

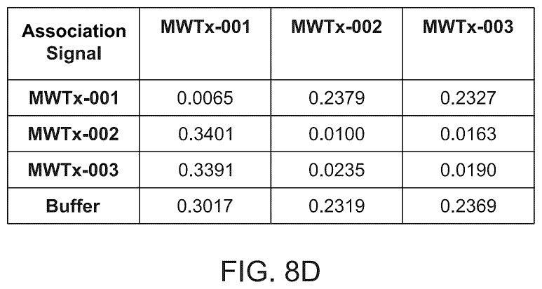

OCTET® RED96e was used for epitope binning of MWTx-001 (FIG. 8A), MWTx-002 (FIG. 8B) and MWTx-003 (FIG. 8C) anti-TMPRSS6 antibodies. First, ecto-TMPRSS6-FLAG (as described above) was labelled with biotin by Biotinylation Kit (Abcam). Pre-hydrated streptavidin (SA) biosensors were equilibrated in 1×KB (as described above) for 60 sec for the first baseline, followed by loading with 10 mg/ml of biotinylated ecto-TMPRSS6-FLAG onto the SA biosensors for 300 sec. Then, the second baseline signal was established for 60 sec before saturation with 50 mg/ml of antibody (MWTx-001, FIG. 8A; MWTx-002, FIG. 8B; MWTx-003, FIG. 8C) in 1×KB for 600 sec. At last, the third baseline signal was established for 60 sec before competition with 50 μg/ml of MWTx-001, MWTx-002 or MWTx-003 in 1×KB for 300 sec. MWTx-001 anti-TMPRSS6 antibody binding towards ecto-TMPRSS6-FLAG was not competed with MWTx-002 anti-TMPRSS6 antibody or MWTx-003 anti-TMPRSS6 antibody (FIG. 8A). MWTx-002 anti-TMPRSS6 antibody binding towards ecto-TMPRSS6-FLAG was not competed with MWTx-001 anti-TMPRSS6 antibody but was competed with MWTx-003 anti-TMPRSS6 antibody (FIG. 8B). MWTx-003 anti-TMPRSS6 antibody binding towards ecto-TMPRSS6-FLAG was not competed with MWTx-001 anti-TMPRSS6 antibody but was competed with MWTx-002 anti-TMPRSS6 antibody (FIG. 8C). Data analysis was done using Octet Data Analysis HT Software. Association signals were summarized in FIG. 8D.

What is claimed is:

1-33. (canceled)

34. An anti-type II transmembrane serine protease 6 (TMPRSS6) antibody comprising:
(a) a heavy chain complementarity determining region (HC CDR) 1 (HC CDR1), a HC CDR2, and a HC CDR3 of a heavy chain variable region (VH) that comprises the amino acid sequence of SEQ ID NO: 1, and a light chain complementarity determining region (LC CDR) 1 (LC CDR1), a LC CDR2, and a LC CDR3 of a light chain variable region (VL) that comprises the amino acid sequence of SEQ ID NO: 6;
(b) a HC CDR1, a HC CDR2, and a HC CDR3 of a VH that comprises the amino acid sequence of SEQ ID NO: 11, and a light chain complementarity determining region (LC CDR) 1 (LC CDR1), a LC CDR2, and a LC CDR3 of a VL that comprises the amino acid sequence of SEQ ID NO: 16;
(c) a HC CDR1, a HC CDR2, and a HC CDR3 of a VH that comprises the amino acid sequence of SEQ ID NO: 31, and a light chain complementarity determining region (LC CDR) 1 (LC CDR1), a LC CDR2, and a LC CDR3 of a VL that comprises the amino acid sequence of SEQ ID NO: 36; or
(d) a HC CDR1, a HC CDR2, and a HC CDR3 of a VH that comprises the amino acid sequence of SEQ ID NO: 41, and a light chain complementarity determining region (LC CDR) 1 (LC CDR1), a LC CDR2, and a LC CDR3 of a VL that comprises the amino acid sequence of SEQ ID NO: 46.

35. The anti-TMPRSS6 antibody of claim 34, wherein the anti-TMPRSS6 antibody comprises:
(a) a HC CDR1 comprising the amino acid of SEQ ID NO: 2, a HC CDR2 comprising the amino acid of SEQ ID NO: 3, a HC CDR3 comprising the amino acid of SEQ ID NO: 4, a LC CDR1 comprising the amino acid of SEQ ID NO: 7, a LC CDR2 comprising the amino acid of SEQ ID NO: 8, a LC CDR3 comprising the amino acid of SEQ ID NO: 9;
(b) a HC CDR1 comprising the amino acid of SEQ ID NO: 12, a HC CDR2 comprising the amino acid of SEQ ID NO: 13, a HC CDR3 comprising the amino acid of SEQ ID NO: 14, a LC CDR1 comprising the amino acid of SEQ ID NO: 17, a LC CDR2 comprising the amino acid of SEQ ID NO: 18, a LC CDR3 comprising the amino acid of SEQ ID NO: 19;
(c) a HC CDR1 comprising the amino acid of SEQ ID NO: 32, a HC CDR2 comprising the amino acid of SEQ ID NO: 33, a HC CDR3 comprising the amino acid of SEQ ID NO: 34, a LC CDR1 comprising the amino acid of SEQ ID NO: 37, a LC CDR2 comprising the amino acid of SEQ ID NO: 38, a LC CDR3 comprising the amino acid of SEQ ID NO: 39; or
(d) a HC CDR1 comprising the amino acid of SEQ ID NO: 42, a HC CDR2 comprising the amino acid of SEQ ID NO: 43, a HC CDR3 comprising the amino acid of SEQ ID NO: 44, a LC CDR1 comprising the amino acid of SEQ ID NO: 47, a LC CDR2 comprising the amino acid of SEQ ID NO: 48, a LC CDR3 comprising the amino acid of SEQ ID NO: 49.

36. The anti-TMPRSS6 antibody of claim 35, wherein the anti-TMPRSS6 antibody comprises:
(a) a VH comprising the amino acid sequence of SEQ ID NO: 1, and a VL comprising the amino acid sequence of SEQ ID NO: 6;
(b) a VH comprising the amino acid sequence of SEQ ID NO: 11, and a VL comprising the amino acid sequence of SEQ ID NO: 16;
(c) a VH comprising the amino acid sequence of SEQ ID NO: 31, and a VL comprising the amino acid sequence of SEQ ID NO: 36; or
(d) a VH comprising the amino acid sequence of SEQ ID NO: 41, and a VL comprising the amino acid sequence of SEQ ID NO: 46.

37. The anti-TMPRSS6 antibody of claim 34, wherein the anti-TMPRSS6 antibody is a full-length antibody, a Fab fragment, or a single-chain variable fragment (scFv).

38. The anti-TMPRSS6 antibody of claim 34, wherein the anti-TMPRSS6 antibody comprises a heavy chain (HC) constant region.

39. The anti-TMPRSS6 antibody of claim 38, wherein the HC constant region is a constant region of human IgG, IgGA, IgGM or IgGE.

40. The anti-TMPRSS6 antibody of claim 39, wherein the HC constant region is a constant region of human IgG1.

41. The anti-TMPRSS6 antibody of claim 34, wherein the anti-TMPRSS6 antibody comprises:
(a) a HC comprising the amino acid sequence of SEQ ID NO: 61, and a light chain (LC) comprising the amino acid sequence of SEQ ID NO: 63;
(b) a HC comprising the amino acid sequence of SEQ ID NO: 65, and a LC comprising the amino acid sequence of SEQ ID NO: 67;
(c) a HC comprising the amino acid sequence of SEQ ID NO: 73, and a LC comprising the amino acid sequence of SEQ ID NO: 75; or
(d) a HC comprising the amino acid sequence of SEQ ID NO: 77, and a LC comprising the amino acid sequence of SEQ ID NO: 79.

42. The anti-TMPRSS6 antibody of claim 34, wherein the anti-TMPRSS6 antibody cross-reacts with at least one non-human TMPRSS6.

43. The anti-TMPRSS6 antibody of claim 42, wherein the non-human TMPRSS6 is a mouse TMPRSS6 or a non-human primate TMPRSS6.

44. The anti-TMPRSS6 antibody of claim 34, wherein the anti-TMPRSS6 antibody specifically binds to human TMPRSS6.

45. The anti-TMPRSS6 antibody of claim 34, wherein the anti-TMPRSS6 antibody does not specifically bind to human matriptase-1 or human matriptase-3.

46. A composition comprising the anti-TMPRSS6 of claim 34 and a pharmaceutically acceptable carrier.

47. An isolated nucleic acid encoding the VH and/or the VL of the anti-TMPRSS6 antibody of claim 34.

48. A vector comprising the isolated nucleic acid of claim 47.

49. A host cell comprising the vector of claim 48.

50. A method of producing an anti-TMPRSS6 antibody, the method comprising:
(i) culturing the host cell of claim 49 under conditions suitable for expressing the anti-TMPRSS6 antibody; and
(ii) harvesting the anti-TMPRSS6 antibody from a host cell lysate or host cell culture supernatant.

51. A method comprising administering to a subject in need thereof the anti-TMPRSS6 antibody of claim 34.

52. A method of treating a subject having a disorder of iron metabolism, the method comprising administering an effective amount of the anti-TMPRSS6 antibody of claim 34 to the subject.

53. The method of claim 52, wherein the disorder of iron metabolism is an iron overload disorder.

54. The method or use of claim 53, wherein the iron overload disorder is β-thalassemia or hereditary hemochromatosis.


SEQUENCE LISTING (102 sequences)

SEQ ID NO: 1 - protein, 118 aa, Mus musculus
QVQLQQPGAELAKPGASVKMSCKASGYTFTSYWITWVKQRPGQDLEWIGNIYPGSGSTYYNEKFKSKATLTVDTSSRTAYMQLSSLTSADSAVYYCAPYDSDYAMDYWGQGTSVTVSS

SEQ ID NO: 2 - protein, 8 aa, Mus musculus
GYTFTSYW

SEQ ID NO: 3 - protein, 8 aa, Mus musculus
IYPGSGST

SEQ ID NO: 4 - protein, 11 aa, Mus musculus
APYDSDYAMDY

SEQ ID NO: 5 - DNA, 354 bp, Mus musculus
  source 1..354: mol_type = other DNA
caggtccaactgcagcagcctggggctgagcttgcgaagcctggggcttcagtgaagatgtcctgcaaggcttctggctacaccttcaccagctactggataacctgggtgaagcagaggcctggacaagaccttgagtggattggaaatatttatcctggtagtggtagtacttactacaatgagaagttcaagagcaaggccacactgactgtagacacatcctccagaacagcctacatgcagctcagcagtctgacatctgcggactctgcggtctattactgtgccccctatgattccgactatgctatggactactggggtcaaggaacctcagtcaccgtctcctca

SEQ ID NO: 6 - protein, 107 aa, Mus musculus
DIKMTQSPSSMYASLGERVTITCKASQDINNYLSWFQQKPGKSPKTLIYRANRLVDGVPSRVSGSGSGQDYSLTISSLEYEDVGIYFCLQYDEFPLTFGAGTKLELK

SEQ ID NO: 7 - protein, 6 aa, Mus musculus
QDINNY

SEQ ID NO: 8 - skipped

SEQ ID NO: 9 - protein, 9 aa, Mus musculus
LQYDEFPLT

SEQ ID NO: 10 - DNA, 321 bp, Mus musculus
  source 1..321: mol_type = other DNA
gacatcaagatgacccagtctccatcttccatgtatgcatctctaggagagagagtcactatcacttgcaaggcgagtcaggacattaataactatttaagctggttccagcagaaaccagggaaatctcctaagaccctgatctatcgtgcaaacagattggtagatggggtcccatcaagggtcagtggcagtggatctgggcaagattattctctcaccatcagcagcctggagtatgaagatgtgggaatttatttttgtctacagtatgatgagtttcctctcacgttcggtgctgggaccaagctggagctgaaa

SEQ ID NO: 11 - protein, 121 aa, Mus musculus
EVQLQQSGAELVKPGASVKLSCTASGFNIKDYYIHWVKERTEQGLEWFGRIDPEDGESEYAPKFQGKATLTADTSSNTAYLQLSSLTSEDTAVYYCTRGDSMMVTYFDYWGQGTTLTVSSE

SEQ ID NO: 12 - protein, 8 aa, Mus musculus
GFNIKDYY

SEQ ID NO: 13 - protein, 8 aa, Mus musculus
IDPEDGES

SEQ ID NO: 14 - protein, 13 aa, Mus musculus
TRGDSMMVTYFDY

SEQ ID NO: 15 - DNA, 360 bp, Mus musculus
  source 1..360: mol_type = other DNA
gaggttcagctgcagcagtctggggcagaacttgtgaagccaggggcctcagtcaagttgtcctgcacagcctctggcttcaacattaaagactactatatacactgggtgaaagagaggactgaacagggcctggagtggtttggaaggattgatcctgaggatggtgaaagtgaatatgccccgaaattccagggcaaggccactttaacagcagacacatcctccaatacagcctacctgcagctcagcagcctgacatctgaggacactgccgtctattactgtactagaggagactctatgatggttacctactttgactactggggccaaggcaccactctcacggtctcctca

SEQ ID NO: 16 - protein, 107 aa, Mus musculus
DIVMTQSHKFMSTSVGDRVSITCKASQDVSTAVAWYQQKPGQSPKLLIYWAFTRHTGVPDRFTSTGSGTDYALTISSVQAEDLALYYCQQHYRSPWTFGGGTKLEIK

SEQ ID NO: 17 - protein, 6 aa, Mus musculus
QDVSTA

SEQ ID NO: 18 - skipped

SEQ ID NO: 19 - protein, 9 aa, Mus musculus
QQHYRSPWT

SEQ ID NO: 20 - DNA, 321 bp, Mus musculus
  source 1..321: mol_type = other DNA
gacattgtgatgacccagtctcacaaattcatgtccacatcagtaggagacagggtcagcatcacctgcaaggccagtcaggatgtgagtactgctgtagcctggtatcaacaaaaaccagggcaatctcctaaactactgatttactgggctttcacccgtcacactggagtccctgatcgcttcacaagcactggatctgggacagattatgctctcaccatcagcagtgtgcaggctgaagacctggcactttattactgtcagcaacattatcgcagtccgtggacgttcggtggaggcaccaaactggaaatcaaa

SEQ ID NO: 21 - protein, 118 aa, Mus musculus
EVQLQQSGAELVKPGASVKLSCTASGFNIEDYYIHWVKERTEQGLEWIGRIDPEDGETTYAPQFQGKATIIPDTSSNTAYMQLSSLTSEDAAVYYCARSIYLDPMDYWGQGTSVTVSS

SEQ ID NO: 22 - protein, 8 aa, Mus musculus
GFNIEDYY

SEQ ID NO: 23 - protein, 8 aa, Mus musculus
IDPEDGET

SEQ ID NO: 24 - protein, 11 aa, Mus musculus
ARSIYLDPMDY

SEQ ID NO: 25 - DNA, 354 bp, Mus musculus
  source 1..354: mol_type = other DNA
gaagttcagctgcagcagtctggggcagaacttgtgaagccaggggcctcagtcaagttgtcctgcacagcttctggcttcaacattgaagactactatatacactgggtgaaggagaggactgaacagggcctggagtggattggaaggattgatcctgaggatggtgaaactacatatgccccgcagttccagggcaaggccactataataccagacacatcctccaacacagcctacatgcagctcagcagcctgacatctgaggacgctgccgtctattactgtgctagatcgatctaccttgatcctatggactactggggtcaaggaacctcagtcaccgtctcctca

SEQ ID NO: 26 - protein, 107 aa, Mus musculus
DIVMTQSHKFMSTSVGDRVSITCKASQDVTTAVAWYQQKPGQSPKILIYWATTRHTGVPDRFTGSISGTTYILTISSVQAEDLALYYCQQHYSTPYTFGGGTKLEIK

SEQ ID NO: 27 - protein, 6 aa, Mus musculus
QDVTTA

SEQ ID NO: 28 - skipped

SEQ ID NO: 29 - protein, 9 aa, Mus musculus
QQHYSTPYT

SEQ ID NO: 30 - DNA, 321 bp, Mus musculus
  source 1..321: mol_type = other DNA
gacattgtgatgacccagtctcacaaattcatgtccacatcagtaggagacagggtcagcatcacctgcaaggccagtcaggatgtgactactgctgtcgcctggtatcaacaaaaaccaggacagtctcctaaaatactgatttactgggcaaccacccggcacactggagtccctgatcgcttcacaggcagtatatctgggacaacttatattctcaccatcagtagtgtgcaggctgaagacctggcactttattactgtcagcaacattatagcactccgtacacgttcggaggggggaccaagctggaaataaaa

SEQ ID NO: 31 - protein, 118 aa, synthetic construct
  REGION 1..118: note = source = /note="Description of Artificial Sequence: Synthetic polypeptide"
EVQLVQSGAEVKKPGASVKVSCKASGYTFTSYWITWVRQAPGQRLEWIGNIYPGSGSTYYNEKFKSKATITRDTSSRTAYMELSSLRSEDTAVYYCAPYDADYAMDYWGQGTLVTVSS

SEQ ID NO: 32 - protein, 8 aa, synthetic construct
  REGION 1..8: note = source = /note="Description of Artificial Sequence: Synthetic peptide"
GYTFTSYW

SEQ ID NO: 33 - protein, 8 aa, synthetic construct
  REGION 1..8: note = source = /note="Description of Artificial Sequence: Synthetic peptide"
IYPGSGST

SEQ ID NO: 34 - protein, 11 aa, synthetic construct
  REGION 1..11: note = source = /note="Description of Artificial Sequence: Synthetic peptide"
APYDADYAMDY

SEQ ID NO: 35 - DNA, 354 bp, synthetic construct
  misc_feature 1..354: note = source = /note="Description of Artificial Sequence: Synthetic polynucleotide"
  source 1..354: mol_type = other DNA
gaagtgcagctggtgcaatctggcgccgaagtgaagaaacctggcgcctctgtgaaggtgtcctgcaaggcttccggctacacctttaccagctactggatcacctgggtccgacaggctcctggccagagactggaatggatcggcaacatctaccctggctccggctccacctactacaacgagaagttcaagtccaaggccacaatcacccgggacacctcttccagaaccgcctacatggaactgtccagcctgagatctgaggacaccgccgtgtactactgcgccccttacgacgccgactacgccatggattattggggccagggcaccctggtcaccgtgtcctct

SEQ ID NO: 36 - protein, 107 aa, synthetic construct
  REGION 1..107: note = source = /note="Description of Artificial Sequence: Synthetic polypeptide"
DIQMTQSPSSLSASVGDRVTITCKASQDISNYLSWFQQKPGKAPKLLIYRANRLVEGVPSRFSGSGSGTDFTLTISSLQPEDFATYFCLQYDEFPLTFGGGTKVEIK

SEQ ID NO: 37 - protein, 6 aa, synthetic construct
  REGION 1..6: note = source = /note="Description of Artificial Sequence: Synthetic peptide"
QDISNY

SEQ ID NO: 38 - skipped

SEQ ID NO: 39 - protein, 9 aa, synthetic construct
  REGION 1..9: note = source = /note="Description of Artificial Sequence: Synthetic peptide"
LQYDEFPLT

SEQ ID NO: 40 - DNA, 321 bp, synthetic construct
  misc_feature 1..321: note = source = /note="Description of Artificial Sequence: Synthetic polynucleotide"
  source 1..321: mol_type = other DNA
gacatccagatgacccagtctccatcctctctgtccgcctctgtgggcgacagagtgaccatcacatgcaaggccagccaggacatctccaactacctgtcctggttccagcagaagcctggcaaggctcccaagctgctgatctacagagccaacagactggtggaaggcgtgccctccagattctccggatctggctctggcaccgactttaccctgacaatctccagcctgcagcctgaggacttcgctacctacttctgcctgcaatacgacgagttccctctgacctttggcggaggcaccaaggtggaaatcaag

SEQ ID NO: 41 - protein, 120 aa, synthetic construct
  REGION 1..120: note = source = /note="Description of Artificial Sequence: Synthetic polypeptide"
EVQLVQSGAEVKKPGASVKVSCKASGFNIKDYYIHWVRQATGQGLEWMGRIDPEDAESEYAPKFQGRVTITADTSTDTAYMELSSLRSEDTAVYYCTRGDSMMVTYFDYWGQGTLVTVSS

SEQ ID NO: 42 - protein, 8 aa, synthetic construct
  REGION 1..8: note = source = /note="Description of Artificial Sequence: Synthetic peptide"
GFNIKDYY

SEQ ID NO: 43 - protein, 8 aa, synthetic construct
  REGION 1..8: note = source = /note="Description of Artificial Sequence: Synthetic peptide"
IDPEDAES

SEQ ID NO: 44 - protein, 13 aa, synthetic construct
  REGION 1..13: note = source = /note="Description of Artificial Sequence: Synthetic peptide"
TRGDSMMVTYFDY

SEQ ID NO: 45 - DNA, 360 bp, synthetic construct
  misc_feature 1..360: note = source = /note="Description of Artificial Sequence: Synthetic polynucleotide"
  source 1..360: mol_type = other DNA
gaagtgcagctggtgcaatctggcgccgaagtgaagaaacctggcgcctctgtgaaggtgtcctgcaaggcctctggcttcaacatcaaggactactacatccactgggtccgacaggctaccggacagggacttgagtggatgggcagaatcgaccctgaggacgccgagtctgagtacgcccctaagtttcagggcagagtgaccatcaccgccgacacctctaccgacaccgcctacatggaactgtccagcctgagatctgaggacaccgccgtgtactactgcaccagaggcgactccatgatggttacctacttcgactactggggccagggcaccctggtcacagtttcttcc

SEQ ID NO: 46 - protein, 107 aa, synthetic construct
  REGION 1..107: note = source = /note="Description of Artificial Sequence: Synthetic polypeptide"
DIQMTQSPSSLSASVGDRVTITCKASQDVSTAVAWYQQKPGKAPKLLIYWAFTRHTGVPSRFSGSGSGTDYALTISSLQPEDFATYYCQQHYRSPWTFGGGTKVEIK

SEQ ID NO: 47 - protein, 6 aa, synthetic construct
  REGION 1..6: note = source = /note="Description of Artificial Sequence: Synthetic peptide"
QDVSTA

SEQ ID NO: 48 - skipped

SEQ ID NO: 49 - protein, 9 aa, synthetic construct
  REGION 1..9: note = source = /note="Description of Artificial Sequence: Synthetic peptide"
QQHYRSPWT

SEQ ID NO: 50 - DNA, 321 bp, synthetic construct
  misc_feature 1..321: note = source = /note="Description of Artificial Sequence: Synthetic polynucleotide"
  source 1..321: mol_type = other DNA
gacatccagatgacccagtctccatcctctctgtccgcctctgtgggcgacagagtgaccatcacatgcaaggcctctcaggacgtgtccaccgccgttgcttggtatcagcagaagcctggcaaggcccctaagctgctgatctactgggccttcaccagacacaccggcgtgccctctaggttctccggctctggctctggcaccgattacgctctgacaatctccagcctgcagcctgaggacttcgccacctactactgccagcagcactacagaagcccctggacatttggcggaggcaccaaggtggaaatcaag

SEQ ID NO: 51 - protein, 118 aa, synthetic construct
  REGION 1..118: note = source = /note="Description of Artificial Sequence: Synthetic polypeptide"
QVQLVQSGAEVKKPGASVKVSCKASGFNIEDYYMHWVRQAPGQRLEWMGRIDPEDAETTYSPKFQGRVTIIPDTSANTAYMELSSLRSEDTAVYYCARSIYLDPMDYWGQGTLVTVSS

SEQ ID NO: 52 - protein, 8 aa, synthetic construct
  REGION 1..8: note = source = /note="Description of Artificial Sequence: Synthetic peptide"
GFNIEDYY

SEQ ID NO: 53 - protein, 8 aa, synthetic construct
  REGION 1..8: note = source = /note="Description of Artificial Sequence: Synthetic peptide"
IDPEDAET

SEQ ID NO: 54 - protein, 11 aa, synthetic construct
  REGION 1..11: note = source = /note="Description of Artificial Sequence: Synthetic peptide"
ARSIYLDPMDY

SEQ ID NO: 55 - DNA, 354 bp, synthetic construct
  misc_feature 1..354: note = source = /note="Description of Artificial Sequence: Synthetic polynucleotide"
  source 1..354: mol_type = other DNA
caggtgcagctggtgcagtctggcgccgaagtgaaaaagcctggcgcctctgtgaaggtgtcctgcaaggcctctggcttcaacatcgaggactactacatgcactgggtccgacaggcccctggccagagattggaatggatgggcagaatcgaccccgaggacgccgagacaacctactctcctaagttccagggccgcgtgacaatcatccctgacacctctgccaacaccgcctacatggaactgtccagcctgagatctgaggacaccgccgtgtactactgcgcccggtctatctacctggaccctatggactattggggccagggcaccctggtcacagtgtcctct

SEQ ID NO: 56 - protein, 107 aa, synthetic construct
  REGION 1..107: note = source = /note="Description of Artificial Sequence: Synthetic polypeptide"
DIQMTQSPKSLSASVGDRVTITCRASQDVTTALAWYQQKPGQSPKLLIYWATTRHSGVPSRFSGSGSGTDFTLTISSLQPEDFATYYCQQHYSTPYTFGQGTKLEIK

SEQ ID NO: 57 - protein, 6 aa, synthetic construct
  REGION 1..6: note = source = /note="Description of Artificial Sequence: Synthetic peptide"
QDVTTA

SEQ ID NO: 58 - skipped

SEQ ID NO: 59 - protein, 9 aa, synthetic construct
  REGION 1..9: note = source = /note="Description of Artificial Sequence: Synthetic peptide"
QQHYSTPYT

SEQ ID NO: 60 - DNA, 321 bp, synthetic construct
  misc_feature 1..321: note = source = /note="Description of Artificial Sequence: Synthetic polynucleotide"
  source 1..321: mol_type = other DNA
gacatccagatgacccagtctccaaagtctctgtccgcctccgtgggcgacagagtgaccatcacctgtagagcctctcaggacgtgaccaccgctctggcttggtatcagcagaagcctggccagtctcctaagctgctgatctactgggccaccaccagacactctggcgtgccctctagattctccggctctggctctggcaccgactttaccctgacaatctccagcctgcagcctgaggacttcgccacctactactgccagcagcactacagcaccccttacacctttggccagggcaccaagctggaaatcaag

SEQ ID NO: 61 - protein, 447 aa, Mus musculus
QVQLQQPGAELAKPGASVKMSCKASGYTFTSYWITWVKQRPGQDLEWIGNIYPGSGSTYYNEKFKSKATLTVDTSSRTAYMQLSSLTSADSAVYYCAPYDSDYAMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 62 - DNA, 1347 bp, Mus musculus
  source 1..1347: mol_type = other DNA
caggtccaactgcagcagcctggggctgagcttgcgaagcctggggcttcagtgaagatgtcctgcaaggcttctggctacaccttcaccagctactggataacctgggtgaagcagaggcctggacaagaccttgagtggattggaaatatttatcctggtagtggtagtacttactacaatgagaagttcaagagcaaggccacactgactgtagacacatcctccagaacagcctacatgcagctcagcagtctgacatctgcggactctgcggtctattactgtgccccctatgattccgactatgctatggactactggggtcaaggaacctcagtcaccgtctcctcagctaaaacaacagccccatcggtctatccactggcccctgtgtgtggagatacaactggctcctcggtgactctaggatgcctggtcaagggttatttccctgagccagtgaccttgacctggaactctggttccctgtccagtggtgtgcacaccttcccagctgtcctgcagtctgacctctacaccctcagctcaagcgtgactgtaaccagctcgacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtagtcgttgatgtgagcgaggatgacccagatgtccagatcagctggtttgtgaacaacgtggaagtgcacactgctcagacacagacgcatagagaggattacaacagtactctccgggttgtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaggagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggagaagaagaactgggtggagagaaatagctactcctgttcagtggtccacgagggtctgcacaatcaccacacgactaagagcttctcccggactccgggttagtaa

SEQ ID NO: 63 - protein, 214 aa, Mus musculus
DIKMTQSPSSMYASLGERVTITCKASQDINNYLSWFQQKPGKSPKTLIYRANRLVDGVPSRVSGSGSGQDYSLTISSLEYEDVGIYFCLQYDEFPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 64 - DNA, 642 bp, Mus musculus
  source 1..642: mol_type = other DNA
gacatcaagatgacccagtctccatcttccatgtatgcatctctaggagagagagtcactatcacttgcaaggcgagtcaggacattaataactatttaagctggttccagcagaaaccagggaaatctcctaagaccctgatctatcgtgcaaacagattggtagatggggtcccatcaagggtcagtggcagtggatctgggcaagattattctctcaccatcagcagcctggagtatgaagatgtgggaatttatttttgtctacagtatgatgagtttcctctcacgttcggtgctgggaccaagctggagctgaaaagagctgacgccgctcctaccgtgtccatctttccacctagcagcgagcagctgacaagcggcggagccagcgtcgtgtgcttcctgaacaacttctaccccaaggacatcaacgtgaagtggaagatcgacggcagcgagagacagaacggcgtgctgaatagctggaccgaccaggacagcaaggactccacctacagcatgtccagcacactgaccctgaccaaggacgagtacgagcggcacaacagctacacatgcgaggccacacacaagaccagcacaagccccatcgtgaagtccttcaaccggaacgagtgc

SEQ ID NO: 65 - protein, 455 aa, Mus musculus
EVQLQQSGAELVKPGASVKLSCTASGFNIKDYYIHWVKERTEQGLEWFGRIDPEDGESEYAPKFQGKATLTADTSSNTAYLQLSSLTSEDTAVYYCTRGDSMMVTYFDYWGQGTTLTVSSKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 66 - DNA, 1365 bp, Mus musculus
  source 1..1365: mol_type = other DNA
gaggttcagctgcagcagtctggggcagaacttgtgaagccaggggcctcagtcaagttgtcctgcacagcctctggcttcaacattaaagactactatatacactgggtgaaagagaggactgaacagggcctggagtggtttggaaggattgatcctgaggatggtgaaagtgaatatgccccgaaattccagggcaaggccactttaacagcagacacatcctccaatacagcctacctgcagctcagcagcctgacatctgaggacactgccgtctattactgtactagaggagactctatgatggttacctactttgactactggggccaaggcaccactctcacggtctcctcaaagaccacacctcctagcgtgtaccctctggctcctggctgtggcgatacaacaggcagctctgtgacactgggctgcctggtcaagggctactttcctgagagcgtgacagtgacctggaacagcggcagcctgtctagcagcgtgcacacctttccagctctgctccagagcggcctgtacaccatgtcctctagtgtgaccgtgcctagcagcacctggcctagccagacagtgacatgtagcgtggcccatcctgccagcagcacaaccgtggacaagaagctggaacctagcggccccatcagcaccatcaatccctgtcctccatgcaaagaatgccacaagtgccccgctcctaacctggaaggtggcccaagcgtgttcatcttcccacctaacatcaaggacgtgctgatgatcagcctgacacctaaagtgacctgcgtggtggtggacgtgtccgaggatgatcccgatgtgcagatcagttggttcgtgaacaacgtggaagtgcacacagcccagacacagacccacagagaggactacaatagcaccattcgcgtggtgtccacactgcctatccagcaccaggattggatgagcggcaaagagttcaagtgcaaagtgaacaacaaggacctgccttctccaatcgagcggaccatcagcaagatcaagggactcgtcagagcccctcaggtgtacatcttgcctccaccagccgagcagctgagcagaaaggatgtgtccctgacctgtctggtcgtgggcttcaaccctggcgacatcagcgtggaatggaccagcaatggccacaccgaggaaaactacaaggacacagcccctgtgctggacagcgacggcagctacttcatctacagcaagctgaacatgaagaccagcaagtgggagaaaaccgacagcttctcctgcaacgtgcggcacgagggcctgaagaactactacctgaagaaaaccatctctcggagccccggcaag

SEQ ID NO: 67 - protein, 214 aa, Mus musculus
DIVMTQSHKFMSTSVGDRVSITCKASQDVSTAVAWYQQKPGQSPKLLIYWAFTRHTGVPDRFTSTGSGTDYALTISSVQAEDLALYYCQQHYRSPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 68 - DNA, 642 bp, Mus musculus
  source 1..642: mol_type = other DNA
gacattgtgatgacccagtctcacaaattcatgtccacatcagtaggagacagggtcagcatcacctgcaaggccagtcaggatgtgagtactgctgtagcctggtatcaacaaaaaccagggcaatctcctaaactactgatttactgggctttcacccgtcacactggagtccctgatcgcttcacaagcactggatctgggacagattatgctctcaccatcagcagtgtgcaggctgaagacctggcactttattactgtcagcaacattatcgcagtccgtggacgttcggtggaggcaccaaactggaaatcaaaagagctgacgccgctcctaccgtgtccatctttccacctagcagcgagcagctgacaagcggcggagccagcgtcgtgtgcttcctgaacaacttctaccccaaggacatcaacgtgaagtggaagatcgacggcagcgagagacagaacggcgtgctgaatagctggaccgaccaggacagcaaggactccacctacagcatgtccagcacactgaccctgaccaaggacgagtacgagcggcacaacagctacacatgcgaggccacacacaagaccagcacaagccccatcgtgaagtccttcaaccggaacgagtgc

SEQ ID NO: 69 - protein, 453 aa, Mus musculus
EVQLQQSGAELVKPGASVKLSCTASGFNIEDYYIHWVKERTEQGLEWIGRIDPEDGETTYAPQFQGKATIIPDTSSNTAYMQLSSLTSEDAAVYYCARSIYLDPMDYWGQGTSVTVSSKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 70 - DNA, 1359 bp, Mus musculus
  source 1..1359: mol_type = other DNA
gaggttcagctgcagcagtctggcgccgagcttgtgaaacctggcgcctctgtgaagctgagctgtaccgccagcggcttcaacatcgaggactactacatccactgggtcaaagagcggaccgagcagggactcgagtggatcggaagaatcgaccccgaggacggcgagacaacatacgcccctcagtttcagggcaaagccacaatcatccccgacaccagcagcaacaccgcctacatgcaactgagcagcctgacctctgaagatgccgccgtgtactactgcgcccggtccatctatctggaccccatggattattggggccagggcacaagcgtgaccgtgtcctctaagaccacacctcctagcgtgtaccctctggctcctggctgtggcgatacaacaggcagctctgtgacactgggctgcctggtcaagggctactttcctgagagcgtgacagtgacctggaacagcggcagcctgtctagcagcgtgcacacctttccagctctgctccagagcggcctgtacaccatgtcctctagtgtgaccgtgcctagcagcacctggcctagccagacagtgacatgtagcgtggcccatcctgccagcagcacaaccgtggacaagaagctggaacctagcggccccatcagcaccatcaatccctgtcctccatgcaaagaatgccacaagtgccccgctcctaacctggaaggtggcccaagcgtgttcatcttcccacctaacatcaaggacgtgctgatgatcagcctgacacctaaagtgacctgcgtggtggtggacgtgtccgaggatgatcccgatgtgcagatcagttggttcgtgaacaacgtggaagtgcacacagcccagacacagacccacagagaggactacaatagcaccattcgcgtggtgtccacactgcctatccagcaccaggattggatgagcggcaaagagttcaagtgcaaagtgaacaacaaggacctgccttctccaatcgagcggaccatcagcaagatcaagggactcgtcagagcccctcaggtgtacatcttgcctccaccagccgagcagctgagcagaaaggatgtgtccctgacctgtctggtcgtgggcttcaaccctggcgacatcagcgtggaatggaccagcaatggccacaccgaggaaaactacaaggacacagcccctgtgctggacagcgacggcagctacttcatctacagcaagctgaacatgaagaccagcaagtgggagaaaaccgacagcttctcctgcaacgtgcggcacgagggcctgaagaactactacctgaagaaaaccatctctcggagccccggcaag

SEQ ID NO: 71 - protein, 214 aa, Mus musculus
DIVMTQSHKFMSTSVGDRVSITCKASQDVTTAVAWYQQKPGQSPKILIYWATTRHTGVPDRFTGSISGTTYILTISSVQAEDLALYYCQQHYSTPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 72 - DNA, 642 bp, Mus musculus
  source 1..642: mol_type = other DNA
gacatcgtgatgacccagagccacaagttcatgagcaccagcgtgggcgacagagtgtccatcacctgtaaagccagccaggacgtgacaacagccgtggcctggtatcagcagaagcctggccagtctcctaagatcctgatctactgggccaccaccagacacaccggcgtgccagatagattcaccggcagcatcagcggcaccacctacatcctgacaatcagctctgtgcaggccgaggatctggccctgtactactgtcagcagcactacagcaccccttacacctttggcggaggcaccaagctggaaatcaagagagctgacgccgctcctaccgtgtccatctttccacctagcagcgagcagctgacaagcggcggagccagcgtcgtgtgcttcctgaacaacttctaccccaaggacatcaacgtgaagtggaagatcgacggcagcgagagacagaacggcgtgctgaatagctggaccgaccaggacagcaaggactccacctacagcatgtccagcacactgaccctgaccaaggacgagtacgagcggcacaacagctacacatgcgaggccacacacaagaccagcacaagccccatcgtgaagtccttcaaccggaacgagtgc

SEQ ID NO: 73 - protein, 447 aa, synthetic construct
  REGION 1..447: note = source = /note="Description of Artificial Sequence: Synthetic polypeptide"
EVQLVQSGAEVKKPGASVKVSCKASGYTFTSYWITWVRQAPGQRLEWIGNIYPGSGSTYYNEKFKSKATITRDTSSRTAYMELSSLRSEDTAVYYCAPYDADYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 74 - DNA, 1341 bp, synthetic construct
  misc_feature 1..1341: note = source = /note="Description of Artificial Sequence: Synthetic polynucleotide"
  source 1..1341: mol_type = other DNA
gaagtgcagctggtgcaatctggcgccgaagtgaagaaacctggcgcctctgtgaaggtgtcctgcaaggcttccggctacacctttaccagctactggatcacctgggtccgacaggctcctggccagagactggaatggatcggcaacatctaccctggctccggctccacctactacaacgagaagttcaagtccaaggccacaatcacccgggacacctcttccagaaccgcctacatggaactgtccagcctgagatctgaggacaccgccgtgtactactgcgccccttacgacgccgactacgccatggattattggggccagggcaccctggtcaccgtgtcctctgcttctaccaagggacccagcgtgttccctctggctccttccagcaagtctacctctggcggaacagctgctctgggctgcctggtcaaggactactttcctgagcctgtgaccgtgtcttggaactctggcgctctgacatccggcgtgcacacatttccagctgtgctgcagtcctccggcctgtactctctgtcctctgtcgtgaccgtgccttcctctagcctgggcacccagacctacatctgcaatgtgaaccacaagccttccaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacgccagcacctacagagtggtgtccgtgctgacagtgctgcaccaggattggctgaacggcaaagagtacaagtgcaaggtgtccaacaaggccctgcctgctcctatcgaaaagaccatcagcaaggccaagggccagcctagagaaccccaggtttacaccttgcctccatctcgggacgagctgaccaagaaccaggtgtccctgacctgtctcgtgaagggcttctacccctccgacatcgccgtggaatgggagtctaatggccagccagagaacaactacaagacaacccctcctgtgctggactccgacggctcattcttcctgtactccaagctgaccgtggacaagtccagatggcagcagggcaacgtgttctcctgctccgtgatgcacgaggccctgcacaatcactacacacagaagtccctgtctctgtcccctggc

SEQ ID NO: 75 - protein, 214 aa, synthetic construct
  REGION 1..214: note = source = /note="Description of Artificial Sequence: Synthetic polypeptide"
DIQMTQSPSSLSASVGDRVTITCKASQDISNYLSWFQQKPGKAPKLLIYRANRLVEGVPSRFSGSGSGTDFTLTISSLQPEDFATYFCLQYDEFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - DNA, 642 bp, synthetic construct
  misc_feature 1..642: note = source = /note="Description of Artificial Sequence: Synthetic polynucleotide"
  source 1..642: mol_type = other DNA
gacatccagatgacccagtctccatcctctctgtccgcctctgtgggcgacagagtgaccatcacatgcaaggccagccaggacatctccaactacctgtcctggttccagcagaagcctggcaaggctcccaagctgctgatctacagagccaacagactggtggaaggcgtgccctccagattctccggatctggctctggcaccgactttaccctgacaatctccagcctgcagcctgaggacttcgctacctacttctgcctgcaatacgacgagttccctctgacctttggcggaggcaccaaggtggaaatcaagcggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgt

SEQ ID NO: 77 - protein, 449 aa, synthetic construct
  REGION 1..449: note = source = /note="Description of Artificial Sequence: Synthetic polypeptide"
EVQLVQSGAEVKKPGASVKVSCKASGFNIKDYYIHWVRQATGQGLEWMGRIDPEDAESEYAPKFQGRVTITADTSTDTAYMELSSLRSEDTAVYYCTRGDSMMVTYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 78 - DNA, 1347 bp, synthetic construct
  misc_feature 1..1347: note = source = /note="Description of Artificial Sequence: Synthetic polynucleotide"
  source 1..1347: mol_type = other DNA
gaagtgcagctggtgcaatctggcgccgaagtgaagaaacctggcgcctctgtgaaggtgtcctgcaaggcctctggcttcaacatcaaggactactacatccactgggtccgacaggctaccggacagggacttgagtggatgggcagaatcgaccctgaggacgccgagtctgagtacgcccctaagtttcagggcagagtgaccatcaccgccgacacctctaccgacaccgcctacatggaactgtccagcctgagatctgaggacaccgccgtgtactactgcaccagaggcgactccatgatggttacctacttcgactactggggccagggcaccctggtcacagtttcttccgcttccaccaagggacccagcgtgttccctctggctccttccagcaagtctacctctggcggaacagctgctctgggctgcctggtcaaggattacttccctgagcctgtgaccgtgtcctggaactctggcgctctgacatccggcgtgcacacctttccagctgtgctgcaatcctccggcctgtactctctgtcctccgtcgtgaccgtgccttctagctctctgggcacccagacctacatctgcaatgtgaaccacaagccttccaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacgcctccacctacagagtggtgtccgtgctgacagtgctgcaccaggattggctgaacggcaaagagtacaagtgcaaggtgtccaacaaggcactgcccgctcctatcgaaaagaccatctccaaggccaagggccagcctagagaaccccaggtttacaccttgcctccatctcgggacgagctgaccaagaaccaggtgtccctgacctgtctcgtgaagggcttctacccctccgacatcgccgtggaatgggagtctaatggccagccagagaacaactacaagacaacccctcctgtgctggactccgacggctcattcttcctgtactccaagctgaccgtggacaagtccagatggcagcagggcaacgtgttctcctgctccgtgatgcacgaggccctgcacaatcactacacacagaagtctctgtccctgtctcctggc

SEQ ID NO: 79 - protein, 214 aa, synthetic construct
  REGION 1..214: note = source = /note="Description of Artificial Sequence: Synthetic polypeptide"
DIQMTQSPSSLSASVGDRVTITCKASQDVSTAVAWYQQKPGKAPKLLIYWAFTRHTGVPSRFSGSGSGTDYALTISSLQPEDFATYYCQQHYRSPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - DNA, 642 bp, synthetic construct
  misc_feature 1..642: note = source = /note="Description of Artificial Sequence: Synthetic polynucleotide"
  source 1..642: mol_type = other DNA
gacatccagatgacccagtctccatcctctctgtccgcctctgtgggcgacagagtgaccatcacatgcaaggcctctcaggacgtgtccaccgccgttgcttggtatcagcagaagcctggcaaggcccctaagctgctgatctactgggccttcaccagacacaccggcgtgccctctaggttctccggctctggctctggcaccgattacgctctgacaatctccagcctgcagcctgaggacttcgccacctactactgccagcagcactacagaagcccctggacatttggcggaggcaccaaggtggaaatcaagcggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgt

SEQ ID NO: 81 - protein, 447 aa, synthetic construct
  REGION 1..447: note = source = /note="Description of Artificial Sequence: Synthetic polypeptide"
QVQLVQSGAEVKKPGASVKVSCKASGFNIEDYYMHWVRQAPGQRLEWMGRIDPEDAETTYSPKFQGRVTIIPDTSANTAYMELSSLRSEDTAVYYCARSIYLDPMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 82 - DNA, 1341 bp, synthetic construct
  misc_feature 1..1341: note = source = /note="Description of Artificial Sequence: Synthetic polynucleotide"
  source 1..1341: mol_type = other DNA
caggtgcagctggtgcagtctggcgccgaagtgaaaaagcctggcgcctctgtgaaggtgtcctgcaaggcctctggcttcaacatcgaggactactacatgcactgggtccgacaggcccctggccagagattggaatggatgggcagaatcgaccccgaggacgccgagacaacctactctcctaagttccagggccgcgtgacaatcatccctgacacctctgccaacaccgcctacatggaactgtccagcctgagatctgaggacaccgccgtgtactactgcgcccggtctatctacctggaccctatggactattggggccagggcaccctggtcacagtgtcctctgcttctaccaagggacccagcgtgttccctctggctccttccagcaagtctacctctggcggaacagctgctctgggctgcctggtcaaggactactttccagagcctgtgaccgtgtcctggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaagctgctggcggcccttccgtgtttctgttccctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggcactgcccgctcctatcgaaaagaccatctccaaggccaagggccagcctagggaaccccaggtttacaccctgcctccaagccgggaagagatgaccaagaaccaggtgtccctgacctgcctcgtgaagggcttctacccttccgacatcgccgtggaatgggagagcaatggccagccagagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacacagaagtccctgtctctgtcccctggc

SEQ ID NO: 83 - protein, 214 aa, synthetic construct
  REGION 1..214: note = source = /note="Description of Artificial Sequence: Synthetic polypeptide"
DIQMTQSPKSLSASVGDRVTITCRASQDVTTALAWYQQKPGQSPKLLIYWATTRHSGVPSRFSGSGSGTDFTLTISSLQPEDFATYYCQQHYSTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - DNA, 642 bp, synthetic construct
  misc_feature 1..642: note = source = /note="Description of Artificial Sequence: Synthetic polynucleotide"
  source 1..642: mol_type = other DNA
gacatccagatgacccagtctccaaagtctctgtccgcctccgtgggcgacagagtgaccatcacctgtagagcctctcaggacgtgaccaccgctctggcttggtatcagcagaagcctggccagtctcctaagctgctgatctactgggccaccaccagacactctggcgtgccctctagattctccggctctggctctggcaccgactttaccctgacaatctccagcctgcagcctgaggacttcgccacctactactgccagcagcactacagcaccccttacacctttggccagggcaccaagctggaaatcaagcggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgt

SEQ ID NO: 85 - DNA, 21 bp, synthetic construct
  misc_feature 1..21: note = source = /note="Description of Artificial Sequence: Synthetic primer"
  source 1..21: mol_type = other DNA
aagcagggcagacattgcgat

SEQ ID NO: 86 - DNA, 21 bp, synthetic construct
  misc_feature 1..21: note = source = /note="Description of Artificial Sequence: Synthetic primer"
  source 1..21: mol_type = other DNA
caggatgtggctctaggctat

SEQ ID NO: 87 - DNA, 20 bp, synthetic construct
  misc_feature 1..20: note = source = /note="Description of Artificial Sequence: Synthetic primer"
  source 1..20: mol_type = other DNA
acccacactgtgcccatcta

SEQ ID NO: 88 - DNA, 20 bp, synthetic construct
  misc_feature 1..20: note = source = /note="Description of Artificial Sequence: Synthetic primer"
  source 1..20: mol_type = other DNA
cacgctcggtcaggatcttc

SEQ ID NO: 89 - protein, 118 aa, synthetic construct
  REGION 1..118: note = source = /note="Description of Artificial Sequence: Synthetic polypeptide"
QVQLVQSGAEVKKPGASVKVSCKASGFNIEDYYMHWVRQAPGQRLEWMGRIDPEDGETTYSPKFQGRVTIIPDTSANTAYMELSSLRSEDTAVYYCARSIYLDPMDYWGQGTLVTVSS

SEQ ID NO: 90 - protein, 118 aa, synthetic construct
  REGION 1..118: note = source = /note="Description of Artificial Sequence: Synthetic polypeptide"
QVQLQQSGAEVKKPGASVKVSCTASGFNIEDYYINWVRQATGQGLEWMGRIDPEDGETGYAQKFQGRVTMTRNTSISTAYMELSSLRSEDTAVYYCARSIYLDPMDYWGQGTLVTVSS

SEQ ID NO: 91 - protein, 118 aa, synthetic construct
  REGION 1..118: note = source = /note="Description of Artificial Sequence: Synthetic polypeptide"
QVQLVQSGAEVKKPGASVKVSCKASGFNIEDYYMHWVRQAPEQGLEWMGRIDPEDGETTYAPKFQGRVTSTRDTSINTAYMELSRLRSDDTVVYYCARSIYLDPMDYWGQGTLVTVSS

SEQ ID NO: 92 - protein, 118 aa, synthetic construct
  REGION 1..118: note = source = /note="Description of Artificial Sequence: Synthetic polypeptide"
QVQLVQSGAEVKKPGASVKVSCKASGFNIEDYYMHWVRQAPEQGLEWMGIIDPEDGETSYAQKFQGRVTMTRDTSTNTAYMELSSLRSEDTAVYYCARSIYLDPMDYWGQGTLVTVSS

SEQ ID NO: 93 - protein, 107 aa, synthetic construct; identical to SEQ ID NO: 56
  REGION 1..107: note = source = /note="Description of Artificial Sequence: Synthetic polypeptide"

SEQ ID NO: 94 - protein, 107 aa, synthetic construct
  REGION 1..107: note = source = /note="Description of Artificial Sequence: Synthetic polypeptide"
DIQMTQSPKSVSASVGDRVTITCRASQDVTTALAWYQQKPGQSPKLLIYWATSLQSGVPSRFSGSGSGTDFTLTISSLQPEDFATYYCQQHYSTPYTFGQGTKLEIK

SEQ ID NO: 95 - protein, 107 aa, synthetic construct
  REGION 1..107: note = source = /note="Description of Artificial Sequence: Synthetic polypeptide"
DIQMTQSPKSLSASVGDRVTITCQASQDVTTALNWYQQKPGKAPKLLIYWATNLETGVPSRFSGSGSGTDFTFTISSLQPEDIATYYCQQHYSTPYTFGQGTKLEIK

SEQ ID NO: 96 - protein, 107 aa, synthetic construct
  REGION 1..107: note = source = /note="Description of Artificial Sequence: Synthetic polypeptide"
DIVMTQSPKSLAVSLGERATINCKSSQDVTTALAWYQQKPGQSPKLLIYWATTRESGVPDRFSGSGSGTDYTLTISSLQAEDVAVYYCQQHYSTPYTFGQGTKLEIK

SEQ ID NO: 97 - protein, 808 aa, synthetic construct
  REGION 1..808: note = source = /note="Description of Artificial Sequence: Synthetic polypeptide"
MPVAEAPQVAGGQGDGGDGEEAEPEGMFKACEDSKRKARGYLRLVPLFVLLALLVLASAGVLLWYFLGYKAEVMVSQVYSGSLRVLNRHFSQDLTRRESSAFRSETAKAQKMLKELITSTRLGTYYNSSSVYSFGEGPLTCFFWFILQIPEHRRLMLSPEVVQALLVEELLSTVNSSAAVPYRAEYEVDPEGLVILEASVKDIAALNSTLGCYRYSYVGQGQVLRLKGPDHLASSCLWHLQGPKDLMLKLRLEWTLAECRDRLAMYDVAGPLEKRLITSVYGCSRQEPVVEVLASGAIMAVVWKKGLHSYYDPFVLSVQPVVFQACEVNLTLDNRLDSQGVLSTPYFPSYYSPQTHCSWHLTVPSLDYGLALWFDAYALRRQKYDLPCTQGQWTIQNRRLCGLRILQPYAERIPVVATAGITINFTSQISLTGPGVRVHYGLYNQSDPCPGEFLCSVNGLCVPACDGVKDCPNGLDERNCVCRATFQCKEDSTCISLPKVCDGQPDCLNGSDEEQCQEGVPCGTFTFQCEDRSCVKKPNPQCDGRPDCRDGSDEEHCDCGLQGPSSRIVGGAVSSEGEWPWQASLQVRGRHICGGALIADRWVITAAHCFQEDSMASTVLWTVFLGKVWQNSRWPGEVSFKVSRLLLHPYHEEDSHDYDVALLQLDHPVVRSAAVRPVCLPARSHFFEPGLHCWITGWGALREGGPISNALQKVDVQLIPQDLCSEVYRYQVTPRMLCAGYRKGKKDACQGDSGGPLVCKALSGRWFLAGLVSWGLGCGRPNYFGVYTRITGVISWIQQVVTHHHHHH

SEQ ID NO: 98 - protein, 817 aa, synthetic construct
  REGION 1..817: note = source = /note="Description of Artificial Sequence: Synthetic polypeptide"
MPRCFQLPCSTRMPTTEVPQAADGQGDAGDGEEAAEPEGKFKPPKNTKRKNRDYVRFTPLLLVLAALVSAGVMLWYFLGYKAEVTVSQVYSGSLRVLNRHFSQDLGRRESIAFRSESAKAQKMLQELVASTRLGTYYNSSSVYSFGEGPLTCFFWFILDIPEYQRLTLSPEVVRELLVDELLSNSSTLASYKTEYEVDPEGLVILEASVNDIVVLNSTLGCYRYSYVNPGQVLPLKGPDQQTTSCLWHLQGPEDLMIKVRLEWTRVDCRDRVAMYDAAGPLEKRLITSVYGCSRQEPVMEVLASGSVMAVVWKKGMHSYYDPFLLSVKSVAFQDCQVNLTLEGRLDTQGFLRTPYYPSYYSPSTHCSWHLTVPSLDYGLALWFDAYALRRQKYNRLCTQGQWMIQNRRLCGFRTLQPYAERIPMVASDGVTINFTSQISLTGPGVQVYYSLYNQSDPCPGEFLCSVNGLCVPACDGIKDCPNGLDERNCVCRAMFQCQEDSTCISLPRVCDRQPDCLNGSDEEQCQEGVPCGTFTFQCEDRSCVKKPNPECDGQSDCRDGSDEQHCDCGLQGLSSRIVGGTVSSEGEWPWQASLQIRGRHICGGALIADRWVITAAHCFQEDSMASPKLWTVFLGKMRQNSRWPGEVSFKVSRLFLHPYHEEDSHDYDVALLQLDHPVVYSATVRPVCLPARSHFFEPGQHCWITGWGAQREGGPVSNTLQKVDVQLVPQDLCSEAYRYQVSPRMLCAGYRKGKKDACQGDSGGPLVCREPSGRWFLAGLVSWGLGCGRPNFFGVYTRVTRVINWIQQVLTHHHHHH

SEQ ID NO: 99 - protein, 806 aa, synthetic construct
  REGION 1..806: note = source = /note="Description of Artificial Sequence: Synthetic polypeptide"
MPVAKAPQVAGGQGDGGDGEEAEPEGMFEACEDSKRKARGYLRLAPLWLTLVVLTSVGVLLWYFLGYKAEVTVSQVYSGSLRVLNRHFSQDLTRRESSAFRSETAKAQKMLKELIASTRLGTYYNSSSVYSFGEGPLTCFFWFILQIPEHRRLMLSPEVVQALLVEELLSTVNSSAAVPYRAEYEVDPEGLVILEASVKDIAALNSTLGCYRYSYVGQGQVLRLKGPDHLASSCLWHLQGPEDLMLKLRLEWTLAECRDRLAMYDVAGPLEKRLITSVYGCSRQEPVVEVLASGAIMAVVWKKGLHSYYDPFMLSVQSVVFQACEVNLTLDDRLDSQGVLSTPYFPSYYSPRTHCSWHLTVPSLDYGLALWFDAYALRRQKYDLPCTQGQWTIQNRRLCGLRILQPYAERIPVVATAGITINFTSQISLTGPGVRVHYGLYNQSDPCPGEFLCSVNGLCVPACDGVKDCPNGLDERNCVCRATFQCQEDSTCISLLKVCDGQPDCLNGSDEERCQEGVPCGTFTFQCEDQSCVKKPNPQCDGRPDCRDGSDEQHCDCGLQGPSSRIVGGAVSSEGEWPWQASLQVRGRHICGGALIADRWVITAAHCFQEDSMASPALWTVFLGKVWQNSRWPGEVSFKVSRLLLHPYHEEDSHDYDVALLQLDHPVVRSAAVRPVCLPARSHFFEPGLHCWITGWGALREGGPTSNALQKVDVQLIPQDLCSEAYRYQVTPRMLCAGYRKGKKDACQGDSGGPLVCKALSGRWFLAGLVSWGLGCGRPNYFGVYTRITGVIGWIQQVVTHHHHHH

SEQ ID NO: 100 - protein, 860 aa, synthetic construct
  REGION 1..860: note = source = /note="Description of Artificial Sequence: Synthetic polypeptide"
MGSDRARKGGGGPKDFGAGLKYNSRHEKVNGLEEGVEFLPVNNVKKVEKHGPGRWVVLAAVLIGLLLVLLGIGFLVWHLQYRDVRVQKVFNGYMRITNENFVDAYENSNSTEFVSLASKVKDALKLLYSGVPFLGPYHKESAVTAFSEGSVIAYYWSEFSIPQHLVEEAERVMAEERVVMLPPRARSLKSFVVTSVVAFPTDSKTVQRTQDNSCSFGLHARGVELMRFTTPGFPDSPYPAHARCQWALRGDADSVLSLTFRSFDLASCDERGSDLVTVYNTLSPMEPHALVQLCGTYPPSYNLTFHSSQNVLLITLITNTERRHPGFEATFFQLPRMSSCGGRLRKAQGTFNSPYYPGHYPPNIDCTWNIEVPNNQHVKVRFKFFYLLEPGVPAGTCPKDYVEINGEKYCGERSQFVVTSNSNKITVRFHSDQSYTDTGFLAEYLSYDSSDPCPGQFTCRTGRCIRKELRCDGWADCTDHSDELNCSCDAGHQFTCKNKFCKPLFWVCDSVNDCGDNSDEQGCSCPAQTFRCSNGKCLSKSQQCNGKDDCGDGSDEASCPKVNVVTCTKHTYRCLNGLCLSKGNPECDGKEDCSDGSDEKDCDCGLRSFTRQARVVGGTDADEGEWWQVSLHALGQGHICGASLISPNWLVSAAHCYIDDRGFRYSDPTQWTAFLGLHDQSQRSAPGVQERRLKRIISHPFFNDFTFDYDIALLELEKPAEYSSMVRPICLPDASHVFPAGKAIWVTGWGHTQYGGTGALILQKGEIRVINQTTCENLLPQQITPRMMCVGFLSGGVDSCQGDSGGPLSSVEADGRIFQAGVVSWGDGCAQRNKPGVYTRLPLFRDWIKENTGVHHHHHH

SEQ ID NO: 101 - protein, 849 aa, synthetic construct
  REGION 1..849: note = source = /note="Description of Artificial Sequence: Synthetic polypeptide"
MDKENSDVSAAPADLKISNISVQVVSAQKKLPVRRPPLPGRRLPLPGRRPPQRPIGKAKPKKQSKKKVPFWNVQNKIILFTVFLFILAVIAWTLLWLYISKTESKDAFYFAGMFRITNIEFLPEYRQKESREFLSVSRTVQQVINLVYTTSAFSKFYEQSVVADVSSNNKGGLLVHFWIVFVMPRAKGHIFCEDCVAAILKDSIQTSIINRTSVGSLQGLAVDMDSVVLNAGLRSDYSSTIGSDKGCSQYFYAEHLSLHYPLEISAASGRLMCHFKLVAIVGYLIRLSIKSIQIEADNCVTDSLTIYDSLLPIRSSILYRICEPTRTLMSFVSTNNLMLVTFKSPHIRRLSGIRAYFEVIPEQKCENTVLVKDITGFEGKISSPYYPSYYPPKCKCTWKFQTSLSTLGIALKFYNYSITKKSMKGCEHGWWEINEHMYCGSYMDHQTIFRVPSPLVHIQLQCSSRLSDKPLLAEYGSYNISQPCPVGSFRCSSGLCVPQAQRCDGVNDCFDESDELFCVSPQPACNTSSFRQHGPLICDGFRDCENGRDEQNCTQSIPCNNRTFKCGNDICFRKQNAKCDGTVDCPDGSDEEGCTCSRSSSALHRIIGGTDTLEGGWPWQVSLHFVGSAYCGASVISREWLLSAAHCFHGNRLSDPTPWTAHLGMYVQGNAKFVSPVRRIVVHEYYNSQTFDYDIALLQLSIAWPETLKQLIQPICIPPTGQRVRSGEKCWVTGWGRRHEADNKGSLVLQQAEVELIDQTLCVSTYGIITSRMLCAGIMSGKRDACKGDSGGPLSCRRKSDGKWILTGIVSWGHGSGRPNFPGVYTRVSNFVPWIHKYVPSLLHHHHHH

SEQ ID NO: 102 - protein, 783 aa, synthetic construct
  REGION 1..783: note = source = /note="Description of Artificial Sequence: Synthetic polypeptide"
MDMRVPAQLLGLLLLWLPGARGADIGYKAEVMVSQVYSGSLRVLNRHFSQDLTRRESSAFRSETAKAQKMLKELITSTRLGTYYNSSSVYSFGEGPLTCFFWFILQIPEHRRLMLSPEVVQALLVEELLSTVNSSAAVPYRAEYEVDPEGLVILEASVKDIAALNSTLGCYRYSYVGQGQVLRLKGPDHLASSCLWHLQGPKDLMLKLRLEWTLAECRDRLAMYDVAGPLEKRLITSVYGCSRQEPVVEVLASGAIMAVVWKKGLHSYYDPFVLSVQPVVFQACEVNLTLDNRLDSQGVLSTPYFPSYYSPQTHCSWHLTVPSLDYGLALWFDAYALRRQKYDLPCTQGQWTIQNRRLCGLRILQPYAERIPVVATAGITINFTSQISLTGPGVRVHYGLYNQSDPCPGEFLCSVNGLCVPACDGVKDCPNGLDERNCVCRATFQCKEDSTCISLPKVCDGQPDCLNGSDEEQCQEGVPCGTFTFQCEDRSCVKKPNPQCDGRPDCRDGSDEEHCDCGLQGPSSRIVGGAVSSEGEWPWQASLQVRGRHICGGALIADRWVITAAHCFQEDSMASTVLWTVFLGKVWQNSRWPGEVSFKVSRLLLHPYHEEDSHDYDVALLQLDHPVVRSAAVRPVCLPARSHFFEPGLHCWITGWGALREGGPISNALQKVDVQLIPQDLCSEVYRYQVTPRMLCAGYRKGKKDACQGDSGGPLVCKALSGRWFLAGLVSWGLGCGRPNYFGVYTRITGVISWIQQVVTGGGGSGGGGSGGGGSDYKDDDDK